(19) United States
(12) Patent Application Publication
(10) Pub. No.: US 2026/0284050 A1
(43) Pub. Date: Sep. 24, 2026

(54) ACTIVATORS OF THE COLD STRESS RESPONSE AND USES THEREOF

(71) Applicants: University of Iceland, Reykjaik (IS); Landspitali University Hospital, Reykjaik (IS)

(72) Inventors: Hans Tómas Björnsson, Seltjarnarnes (IS); Salvör Rafnsdóttir, Kópavogur (IS)

(21) Appl. No.: 19/473,540

(22) PCT Filed: Apr. 12, 2024
(86) PCT No.: PCT/EP2024/060020

§ 371 Date: Oct. 8, 2025

(30) Foreign Application Priority Data
Apr. 12, 2023 (EP) 23167505.9

(51) Int. Cl. A61K 31/517; A61K 31/277; A61P 25/00; C07K 14/47; C12N 15/86; C12Q 1/6851; G01N 21/64; G01N 33/50

(52) U.S. Cl. CPC A61K 31/517; A61K 31/277; A61P 25/00; C07K 14/4703; C07K 14/4705; C12N 15/86; C12Q 1/6851; G01N 21/6486; G01N 33/5023; C07K 2319/60; C12N 2740/15043; G01N 2500/10

(57) ABSTRACT

The present invention relates to compounds capable of activating a cold stress response in an individual in need thereof, as well as their uses.

### TECHNICAL FIELD

The present invention relates to compounds capable of activating a cold stress response in an individual in need thereof, without actually lowering the body temperature of said individual, as well as their uses.

### BACKGROUND

Targeted Temperature Management (TTM) is a common therapeutic strategy in clinical practice to minimize neurological damage following neonatal asphyxia and cardiac arrest (Mathew et al. 2022; Luedke et al. 2022). The reasons why lowered internal core temperature (32° C.-36° C.) yields benefits are currently not fully understood. Although some argue that the positive neurological benefit from mild hypothermia relates to lower metabolic rate, others point out that upregulation of several genes during mild hypothermia does not fit that idea.

An alternative hypothesis is that hypothermia is beneficial, because of the activation of the mild hypothermia stress response itself and if so it would be important to understand the extent of the response. In support of this, researchers have found that several genes (e.g., C/RBP/CIRP, RBM3, SP1) consistently show increased expression during mild hypothermia; two of these, SP1 and RBM3, appear to have roles in decreasing neuronal death during disease or condition states (Zhu et al. 2016; Ryu et al. 2003).

Scientists have recently found that SP1, a transcription factor known to bind at the promoter of many genes, binds to the promoter region of CIRBP at a sequence called Mild Cold Response Element (MCRE), and its binding leads to increased expression of CIRBP at 32° C., indicating that regulation occurs at the transcriptional level (Wierstra 2008; Sumitomo et al. 2012). Similarly, RBM3 is known to regulate at least one downstream gene, RTN3, a potential neuroprotective factor in two different neurodegeneration models (Bastide et al. 2017). Although this suggests that mammalian cells have a pathway culminating in SP1/CIRBP and RBM3/RTN3, little has been done to further elucidate the upstream regulation of this pathway and relatively little knowledge is available regarding mammalian cellular responses to cooling compared to heat shock responses, which have been previously extensively characterized.

### SUMMARY

The inventors of the present disclosure used complementary and unbiased strategies to gain clues into the mild hypothermia response upstream of SP1 and RBM3. Specifically, the inventors developed an array of mild hypothermia indicator (MHI) constructs and identified some FDA approved drugs as being capable of inducing a mild hypothermia response in an individual, without actually inducing hypothermia in said individual.

Thus, one aspect of the present disclosure concerns a composition comprising a compound selected from the group consisting of: Entacapone and Poziotinib, or a pharmaceutically acceptable salt thereof for use in the treatment of a disease or condition selected from the group consisting of: neonatal asphyxia, cerebral haemorrhage, subarachnoid haemorrhage (SAH), subdural hematoma (SH), cerebral infarction, cerebral vasospasm, in an individual in need thereof.

In another aspect, the present disclosure concerns a composition comprising a compound selected from the group consisting of: Entacapone and Poziotinib, or a pharmaceutically acceptable salt thereof for use in the treatment of a disease or condition selected from the group consisting of: stroke, ischemia, cardiac arrest, migraine, traumatic brain injury, recovery after intense exercise, drug overdose, spinal injury, and non-accidental paediatric abusive head trauma, brachial plexopathy, hair loss and neurodegeneration, in an individual in need thereof.

In another aspect, the present disclosure concerns a for use in decreasing neuronal death in an individual in need thereof, wherein the composition comprises a compound selected from the group consisting of: Entacapone and Poziotinib, or a pharmaceutically acceptable salt thereof, and wherein the individual suffers from a disease or condition selected from the group consisting of: neonatal asphyxia, stroke, ischemia, cardiac arrest such as ventricular fibrillation cardiac arrest, migraine, traumatic brain injury, recovery after intense exercise, drug overdose, cerebral haemorrhage, subarachnoid hemorrhage (SAH), subdural hematoma (SH), cerebral infarction, cerebral vasospasm, spinal injury, neonatal abusive head trauma, brachial plexopathy, and, hair loss.

In another aspect, the present disclosure concerns a method of inducing a mild hypothermia response in an individual in need thereof, said method comprising inhibiting SMYD5 and/or inhibiting BAZ2A in said individual.

In another aspect, the present disclosure concerns a mild hypothermia indicator (MHI) construct comprising:
a) a promoter sequence of a gene selected from the group consisting of SP1, RBM3 and CIRBP; and
b) a nucleic acid sequence encoding a fluorescent protein.

In a further aspect, the present disclosure concerns an in vitro method for monitoring activation of the mild hypothermia response in a biological sample in response to a treatment, the method comprising the steps of:
a. providing a first biological sample obtained from a mammal before administration of the treatment,
b. contacting the first biological sample with an MHI construct comprising
   i. a promoter sequence of a gene selected from the group consisting of SP1, RBM3 and CIRBP; and
   i. a nucleic acid sequence encoding a detectable protein, wherein the detectable protein is selected from the group consisting of a fluorescent protein and a luminescent protein;
c. determining the level of fluorescence or luminescence in the first biological sample,
d. providing a second biological sample obtained from the same mammal after administration of the treatment,
e. contacting the second biological sample with an MHI according to b.,
f. determining the level of fluorescence or luminescence in the second biological sample,

wherein the mild hypothermia response is activated by the treatment if the fluorescence in the second biological sample is higher than in the first biological sample.

In an even further aspect, the present disclosure concerns a method for monitoring activation of a mild hypothermia response in an individual following administration of a treatment, the method comprising the steps of:
a) quantifying the amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in a first sample, wherein said first sample is obtained from the individual before said treatment;
b) quantifying the amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in a second sample obtained from the same individual as in a), wherein said second sample is obtained after administration of said treatment; and
c) comparing the amount of a protein quantified in a) and b);
d) concluding that the mild hypothermia response is activated by the treatment if the amount of the protein quantified in b) is lower than in a), wherein a lower amount of protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in b) than in a) is indicative of that the treatment activates the mild hypothermia response.

In an even further aspect, the present disclosure concerns a method for evaluating the effect of a candidate pharmacological agent for treatment of a disease or condition selected from the group consisting of: neonatal asphyxia, stroke, ischemia, cardiac arrest, migraine, traumatic brain injury, recovery after intense exercise, drug overdose, cerebral haemorrhage, subarachnoid haemorrhage (SAH), subdural hematoma (SH), cerebral infarction, cerebral vasospasm, spinal injury, neonatal abusive head trauma, brachial plexopathy, hair loss due to chemotherapy, chronic pain, neuropathic pain, and neurological pain, in an individual in need thereof, the method comprising the steps of:
a) providing mammalian cells transfected with a MHI construct comprising
   ii. a promoter sequence of a gene selected from the group consisting of SP1, RBM3 and CIRBP; and
   ii. a nucleic acid sequence encoding a detectable protein, wherein the detectable protein is selected from the group consisting of a fluorescent protein and a luminescent protein;
b) measuring fluorescence or luminescence of the cells,
c) contacting the cells with a candidate pharmacological agent,
d) measuring fluorescence or luminescence of the cells after exposure to the candidate pharmacological agent,

wherein a higher fluorescence or luminescence of the cells after exposure to the candidate pharmacological agent is indicative of the agent having an effect for treatment of said disease or condition.

### DESCRIPTION OF DRAWINGS

FIG. 1: MHIs based on all the genes SP1, CIRBP, RBM3 showed strongest effect at 32° C. (A) A schematic of the structure of the MHI constructs. (B-D) Representative images of individual SP1-MHIs at 37° C. (E-H) Western blot demonstrating increased SP1, RBM3 and CIRBP at 32° C. compared to 37° C. Shown are data points, each data point a biological replicate, mean and SD where applicable. Significance levels calculated with unpaired one-tailed t-test in GraphPad Prism. (I-J) Mean fluorescence (FACS) of SP1-MHI and CIRBP-MHI (16 h at 32° C., 37° C., 40° C.). Shown are data points, each point a technical replicate, mean and SD where applicable. Significance levels calculated in GraphPad Prism with Sidek's multiple comparison test. (K) Mean fluorescence (FACS) of RBM3-MHI (16 h at 32° C. and 37° C.). Data shown as in (I-J), significance level calculated with unpaired one-tailed t-test in GraphPad Prism. (L-M) No increased fluorescence was observed at moderate hypothermia for either SP1-MHI or CIRBP-MHI. (N) Fluorescence measured by FACS by removing samples after different time to estimate activation curve for the SP1-MHI. Data shown as in (I-J) (O-Q) Fluorescence measured hourly using an IncucyteS3 at 32° C. using MHI-SP1, MHI-CIRBP, MHI-RBM3 respectively. *=P<0.05, **=P<0.01, ***=P<0.001, ****=P<0.0001. Light grey indicates 32° C., dark grey indicates 37° C., black indicates 40° C.

FIG. 2: Poziotinib and Entacapone activate the mild hypothermia response. (A) A schematic regarding the drug screen in HEK293FT transfected with SP1+MCRE-MHI: the cells are transfected, then a drug is administered, and fluorescence is measured. (B) Partial list of drugs found to cause more than >3.75 times increase in fluorescence of the SP1+MCRE-MHI. (C-D) Mean fluorescence of SP1-MHI as measured by FACS after 16 h of either 32° C. or 37° C. with or without Entacapone (C) and Poziotinib (D) exposure. Shown are data points: each is a technical replicate, mean and SD where applicable. Significance levels calculated with Sidek's multiple comparison test in GraphPad Prism. (E-J) Relative mRNA expression of SP1 (E), CIRBP (F, 1), RBM3 (G, J), when compared to GAPDH after 16 h at 32° C. or 37° C. with and without Entacapone (E-G) or Poziotinib (H-J). Shown are data points, each data point is a biological replicate (mean of technical replicates), mean and SD where applicable. Significance levels calculated with unpaired one tailed Student t test in GraphPad Prism. (K) Western blot of SP1, RBM3 and CIRBP, after exposure to Entacapone for 24 h. Data shown as in (G-L). *=P<0.05, **=P<0.01, ***=P<0.001, ****=P<0.0001.

FIG. 3. Genome wide CRISPR-Cas9 Knock Out screen on SP1- and RBM3-MHIs reveals multiple potential inhibitors/activators of the mild hypothermia response. (A) Fluorescence and sort gates of 4 replicates of GeCKO-ed HEK293WT+Cas9+SP1 cells, negative control (HEK293WT) and positive control (HEK293WT+Cas9+SP1). (B) Fluorescence and sort gates of 4 replicates of GeCKO-ed HEK293WT+Cas9+RBM3 cells, or negative control (HEK293WT) and positive control (HEK293WT+Cas9+RBM3). (C) A summary of 19 shared repressors and their −Log 10 (RRA score). *=P<0.05, **=P<0.01, ***=P<0.001, ****=P<0.0001.

FIG. 4: (A-B) Overexpressed FLAG-tagged SMYD5 in mouse embryonic stem cells, binds at promoters of SP1 (A) and CIRBP (B). (C-D) Venn graphs showing upregulated (C) and downregulated (D) SMYD5-bound genes in SMYD5-KO cells. (E) RT-qPCR from SMYD5 KO cells. (F-1) Western blot and quantification using antibodies against SP1, CIRBP and RBM3 in SMYD5 KO cells. *=P<0.05, **=P<0.01, ***=P<0.001, ****=P<0.0001.

FIG. 5: SMYD5 is degraded at 32° C. in vitro and in vivo. (A) Strong intensity of endogenous SMYD5 after exposure to 37° C. but not 32° C. for 6 hours. (B) Quantification of SMYD5 intensity among replicates normalized to DAPI after 6 hours at 37° C. and 32° C. (C) SMYD5 amounts in Western blot at 37° C. and 32° C. after 6 hours. (D) Proteasome inhibitor (MG132) increases levels of SMYD5 at 32° C. to levels seen at 37° C. after 6 hour exposure. (E) qRT-PCR results for SMYD5 at 37° C. and 32° C. (F) and (G) Low SMYD5 levels in vivo in cortex and hippocampus of mice cooled with a standard targeted temperature protocol. *=P<0.05, **=P<0.01, ***=P<0.001, ****=P<0.0001.

FIG. 6: H3K36me3 levels show the same pattern with SMYD5-KD at 37° C. and SMYD5-WT at 32° C. in the HEK293 cell line. Lower H3K4me3 levels at 32° C. compared to 37° C. in both hNPC (A) SMYD5-WT, and HEK293 (B) both SMYD5-WT and SMYD5-KD. (C) Lower levels of H3K36me3 at 32° C. compared to 37° C. in hNPC. (D) SMYD5-KD in HEK293 at 37° C. shows the lowest H3K36me3 levels. There is no difference in H3K36me3 modification levels that are observed with SMYD5-WT and −KD at 32° C. (E) We examined the mean Log 2FoldChange (Log 2FC) of H3K36me3 signal over promoter regions for each gene with significantly differential decreased H3K36me3 modifications at 32° C. When comparing this metric for two datasets, SMYD5-KD versus -WT at 37° C. and SMYD5-WT at 32° C. vs 37° C., we observe that 84% of the genes with decreased H3K36me3 at 32° C. in SMYD5-WT also have decreased H3K36me3 with SMYD5-KD at 37° C. (left). In contrast, we do not observe this same phenomenon for other regions such as distal intergenic regions (right). Up and down arrows show where the majority of H3K36me3 marked genes are distributed. Each gene is depicted in grey, if many genes overlap the dots become more condensed and show a darker colour at their overlapping areas.

FIG. 7. Overlap between all upregulated genes with cold stimulus and known SMYD5 regulated genes. A) A summary of RNA-Seq DEGs results for upregulated genes from three sources (mNPCs, mouse hippocampus, mouse cortex). B) A summary of RNA-Seq DEGs results for down-regulated genes from same three sources as in A. C) Significant overlap of up-regulated genes from three sources and SMYD5-regulated genes defining 37 genes. D), E) and F) Behavior of the 37 genes in all three sources (mNPC, Cortex and Hippocampus, respectively) where they generally show upregulation in all three sources.

### DETAILED DESCRIPTION

### Definitions

As used herein, the singular forms “a”, “an” and “the” include plural referents unless the context clearly states otherwise. Thus, for example, reference to “an indicator” includes a plurality of such indicators. Similarly, “a mild hypothermia indicator” or “MHI” can also refer to “mild hypothermia indicators” or “MHIs”, as for example the indicators described in Examples 1 to 8, and functional variants thereof.

As used herein, the term “some embodiments” can include one, or more than one embodiment.

As used herein “expression vector” or “vector” refers to a DNA construct containing a DNA sequence that is operably linked to a suitable control sequence capable of effecting the expression of the DNA in a suitable host. Such control sequences may, e.g., include a promoter to effect transcription, an optional operator sequence to control such transcription, a sequence encoding suitable mRNA ribosome binding sites and sequences which control termination of transcription and translation. The vector may, e.g., be a plasmid, a phage or simply a potential genomic insert. Once transformed into a suitable host, the vector may, e.g., replicate and function independently of the host genome, or may, in some instances, integrate into the genome itself. Expression vectors are designed, for example, as described in Li et al. (Construction strategies for developing expression vectors for recombinant monoclonal antibody production in CHO cells, Mol Biol Rep. 2018 December; 45(6):2907-2912.

A “sample” or “biological sample” as used herein encompasses any subject and a variety of sample types obtained from any subject. Examples of samples useful in the disclosed methods include but are not limited to a subject, a liquid tissue sample such as blood, or a solid tissue sample such as biopsy material or tissue cultures or cells derived there from and the progeny thereof. For example, biological samples include cells obtained from a tissue sample collected from a subject. Thus, samples encompass clinical samples, cells in culture, cell supernatants, cell lysates, and tissue samples.

By “treatment” we include both therapeutic and prophylactic or preventive treatment of the patient. The terms ‘preventive’ or ‘prophylactic’ are used to encompass the use of a composition or compound disclosed herein, or formulation thereof, which either prevents or reduces the likelihood of a neuronal damage, neurodegeneration, and any consequence that hypoxia may cause to organs and/or tissues in a patient or subject. “Treatment” and “alleviation” are used interchangeably herein.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e., a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit or else several smaller dose units and also by multiple administrations of subdivided doses at specific intervals. Alternatively, the does may be provided as a continuous infusion over a prolonged period.

The term “Individual” or “subject” refers to vertebrates, particular members of the mammalian species, preferably primates including humans. As used herein, ‘subject’ and ‘individual’ may be used interchangeably.

It will be understood by one of ordinary skill in the art that variants of the nucleic acid sequences comprised in the MHIs of the present disclosure, and variations of the encoded polypeptides, are also contemplated in some aspects of the present disclosure. Variants of the MHIs can include homologs. A homolog of a MHI of the present disclosure is a nucleic acid sequence from a human or other animal that has a high degree of structural similarity to an identified MHI of the present disclosure, e.g., at least about 75%, 80%, 85%, 90%, 95% or more sequence identity. Identification of human and other organism homologs of MHIs will be familiar to those of skill in the art. In general, nucleic acid hybridization is a suitable method for identification of homologous sequences of another species (e.g., human, cow, sheep), that correspond to a known sequence. Moreover, the variants encompassed by the present disclosure are so-called “functional variants” of a MHI of the present disclosure. Thus, these variants or functional variants are a nucleic acid sequences capable of performing essentially the same functions as the MHIs of the present disclosure. In particular, a functional variant can respond the mild hypothermia and to agents activating a mild hypothermia response as the MHIs they are a functional variant of.

Sequence identity can be calculated using various, publicly available software tools developed by NCBI (Bethesda, Md.) that can be obtained through the Internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis can be obtained using the MacVector sequence analysis software (Oxford Molecular Group).

The presently disclosed subject matter also includes degenerate nucleic acids that include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating fusion protein reporter polypeptide. Similarly, nucleotide sequence triplets which encode other amino acid residues include, but are not limited to: CCA, CCC, CCG, and CCT (proline codons); CGA, CGC, CGG, CGT, AGA, and AGG (arginine codons); ACA, ACC, ACG, and ACT (threonine codons); AAC and AAT (asparagine codons); and ATA, ATC, and ATT (isoleucine codons). Other amino acid residues may be encoded similarly by multiple nucleotide sequences. Thus, the presently disclosed subject matter embraces degenerate nucleic acids that differ from the biologically isolated nucleic acids in codon sequence due to the degeneracy of the genetic code.

The presently disclosed subject matter also provides modified nucleic acid molecules, which include additions, substitutions and deletions of one or more nucleotides (preferably 1-20 nucleotides) that are useful for practicing the presently disclosed subject matter. As used herein the terms: “deletion,” “addition,” and “substitution,” mean deletion, addition, and substitution changes to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more nucleic acids of a sequence of the presently disclosed subject matter. These modified nucleic acid molecules and/or the polypeptides they encode are functional variants of the MHIs and thus retain at least one activity or function of the unmodified nucleic acid molecule and/or the polypeptides disclosed herein, such as responding to mild hypothermia and a mild hypothermia or cold stress response, such as being activated when exposed to mild hypothermia or a mild hypothermia or cold stress response. In some embodiments, the modified nucleic acid molecules encode modified polypeptides, preferably polypeptides having conservative amino acid substitutions. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art. For example, modified nucleic acid molecules that encode polypeptides having single amino acid changes can be prepared. Each of these nucleic acid molecules can have one, two, or three nucleotide substitutions exclusive of nucleotide changes corresponding to the degeneracy of the genetic code as described herein. Any of the foregoing nucleic acids or polypeptides can be tested by routine experimentation for retention of structural relation or activity to the nucleic acids and/or polypeptides disclosed herein.

As used herein, the terms “increase,” “decrease,” and “difference”, normally referred to fluorescence and/or luminescence, preferably mean significant increase, decrease, and difference respectively, e.g. statistically significant.

The term “mild hypothermia response” or “mammalian hypothermia” or “cold stress response” or “mild cold response” referred to an individual, for example a mammal, is used herein to indicate all those metabolic and physiological changes that occur into a mammal individual, when the body temperature of said individual has been actively reduced to the range of 32° C. to 36° C. One of the aspects of the present disclosure relates to induction of mild hypothermia response in an individual, without reducing the body temperature below 36° C. Other terms that have been used in the art to refer to the mild hypothermia response are “cold shock response” (see e.g., Fujita, 1999), or just “cold shock” (e.g., see Bastide et al. 2017).

The term “Targeted Temperature Management”, “therapeutic hypothermia”, “hypothermia therapy” or “protective hypothermia” refers to an active treatment that tries to achieve and maintain a specific body temperature, normally in the range of 32° C. to 36° C., in an individual for a specific duration of time. For example, this treatment may be helpful to improve health outcomes during recovery after a period of stopped blood flow to the brain, such as after a cardiac arrest, or after the blockage of an artery by a clot as in the case of a stroke, such as in the case of Neonatal encephalopathy to improve the outcome for newborn infants affected by perinatal hypoxia-ischemia, such as in consequence of other medical conditions. “Targeted Temperature Management”, “therapeutic hypothermia”, “hypothermia therapy” or “protective hypothermia” may also comprise a phase during which the body temperature is raised back to normal, i.e. 36-38° C.

The term “inducer of the Mild Cold Response” as defined herein refers to any compound, protein, or genetic element capable of inducing or promoting the mild hypothermia response in an individual. Such condition is readily determined or known to those skilled in the art. Said condition can be for example readily determined by detecting an increase in the transcription of at least one gene selected from the group consisting of: SP1, CIRBP, RBM3.

The terms “MHI”, Mild Hypothermia Indicators, or “indicator” as defined herein are used interchangeably and refer to indicator constructs that encode a protein reporter when the mild hypothermia response is active/activated.

As used herein, the term “reporter” or equivalent terms refers in a general sense to any component that can be readily detected in a system under study, where the detection of the reporter correlates with the presence or absence of some other molecule or property, in particular with exposure to mild hypothermia and/or to compounds, e.g. drugs or pharmacological agents, that induce a mild hypothermia response or cold stress response in the system of interest. The choice of the most suitable reporter to be used for a particular application depends on the intended use, and other variables known to one familiar with the art. In some aspects, a reporter is a reporter gene.

### Therapeutic Indications

The present disclosure provides a composition useful for treatment of any disease or condition that would benefit from induction of mild hypothermia response. For example, the present disclosure provides a composition that can be used to treat any disease or condition that is normally treated with Targeted temperature Management (TTM) therapy and/or hypothermia therapy.

Thus, one aspect of the present disclosure relates to a composition for use in treating a diseases or condition selected from the group consisting of: neonatal asphyxia; stroke; ischemia; cardiac arrest, such as ventricular fibrillation cardiac arrest; migraine; traumatic brain injury; recovery after intense exercise; drug overdose; cerebral haemorrhage; subarachnoid hemorrhage (SAH); subdural hematoma (SH); cerebral infarction; cerebral vasospasm; spinal injury; neonatal abusive head trauma; brachial plexopathy; hair loss due to chemotherapy; chronic pain; neuropathic pain; and neurological pain, in an individual in need thereof, wherein the composition comprises a compound selected from the group consisting of a catechol-O-methyltransferase inhibitor, such as Entacapone, Nebicapone, Nitecapone, Opicapone, Tolcapone; and a tyrosine kinase inhibitor, such as Poziotinib.

Another aspect of the present disclosure is directed to a composition for use in decreasing neuronal death in an individual in need thereof, wherein the composition comprises a compound selected from the group consisting of a catechol-O-methyltransferase inhibitor, such as Entacapone, Nebicapone, Nitecapone, Opicapone, Tolcapone; and a tyrosine kinase inhibitor, for example a pan-her inhibitor (an inhibitor of her-1, her-2 and her-3, such as Poziotinib, afatinib, mobocertinib, amivantamab, Pyrotinib, sapatinib, erlotinib, dacomitinib, neratinib, cancertinib, varlitinib and wherein the individual suffers from a disease or condition selected from the group consisting of: neonatal asphyxia, stroke, ischemia, cardiac arrest such as ventricular fibrillation cardiac arrest, migraine, traumatic brain injury, recovery after intense exercise, drug overdose, cerebral haemorrhage, subarachnoid haemorrhage (SAH), subdural hematoma (SH), cerebral infarction, cerebral vasospasm, spinal injury, neonatal abusive head trauma, brachial plexopathy, and, hair loss after chemotherapy.

In some embodiments, said disease or condition is selected from the group consisting of: neonatal asphyxia; stroke; ischemia; cardiac arrest, such as ventricular fibrillation cardiac arrest; headache, such as migraine; traumatic brain injury; recovery after intense exercise; drug overdose; cerebral haemorrhage; subarachnoid haemorrhage (SAH); subdural hematoma (SH); cerebral infarction; cerebral vasospasm; spinal injury; neonatal abusive head trauma; brachial plexopathy; and hair loss.

In some embodiments, said disease or condition is selected from the group consisting of: neonatal asphyxia; stroke; ischemia; cardiac arrest; migraine; traumatic brain injury; recovery after intense exercise; cerebral haemorrhage; subarachnoid haemorrhage (SAH); subdural hematoma (SH); cerebral infarction; cerebral vasospasm; spinal injury; and neonatal abusive head trauma.

In some embodiments, said disease or condition is selected from the group consisting of: neonatal asphyxia, cerebral haemorrhage, subarachnoid haemorrhage (SAH), subdural hematoma (SH), cerebral infarction, cerebral vasospasm, in an individual in need thereof.

In some embodiments, said disease or condition is selected from the group consisting of: stroke; ischemia; and cardiac arrest.

In some embodiments, said disease or condition is ischemia. For example, the disease or condition may be cardiac ischemia, brain ischemia or liver ischemia. Hypothermia is known to be beneficial to a patient suffering from an ischemic attack as it reduces the risk of hypoxia in the affected organ and may protect that organ from reperfusion injuries (Wang et al. 2005).

In some embodiments, said disease or condition is stroke. For example, the disease or condition may be an ischemic stroke or a haemorrhagic stroke. A positive effect of induced hypothermia for prevention and treatment of neurological injuries e.g. as consequence of stroke or haemorrhagic stroke is shown in Polderman 2008.

In some embodiments, said disease or condition is cardiac arrest A positive effect of temperature management therapy for prevention and treatment of neurological injuries e.g. as consequence of stroke or haemorrhagic stroke is shown in Luedke et al. 2022.

In some embodiments, said disease or condition is ventricular fibrillation cardiac arrest.

In some embodiments, said disease or condition is neonatal asphyxia. Neonatal asphyxia or birth asphyxia may occur when a baby's brain and other organs do not get enough oxygen and nutrients before, during or right after birth. Neonatal asphyxia may be mild, moderate or severe. A diagnosis of mild or moderate asphyxia is normally given when normal respiration is not established within one minute from birth, but heart rate is 100 or above, some muscle tone is present and some response to stimulation is present; this is sometimes referred to as “blue asphyxia”. A diagnosis of severe asphyxia is normally given when a pulse of less than 100 per minute is observed at birth and then it is either falling or steady, respiration is absent or gasping, colour is poor and tone is absent; this is sometimes referred to as “white asphyxia”. Current gold-standard for the treatment of for neonatal asphyxia is using targeted temperature management (Mathew et al. 2022). However, this treatment has only been successful in Western tertiary hospital settings (Thayyil et al. 2021).

In some embodiments, said disease or condition is intrauterine hypoxia, which may occur when a baby's brain and other organs do not get enough oxygen and nutrients before birth.

In some embodiments, said disease or condition is selected from the group consisting of: chronic pain from rheumatic or degenerative origins; neuropathic pain; and other neurological pain such as migraine.

Several studies have demonstrated that cold intervention, such as cryotherapy, hypothermia treatment or TTM, are beneficial for pain relive, for example in case of migraine (Hsu et al. 2022), and chronic pain (Garcia et al. 2021), as well as pain that occurs in individuals as result of injuries that effect the neural system, such as spinal cord injuries (Eller et al. 2022).

Thus, in some embodiments, said disease or condition is ischemia. In some embodiments, said disease or condition is headache, such as migraine or cluster headache syndrome. In some embodiments, said disease or condition is migraine. In some embodiments, said disease or condition is traumatic brain injury, such as severe brain injury.

In some embodiments, said disease or condition occurs as a consequence of a traumatic brain injury in an adult or in a baby or infant, including a birth injury. For example, in some embodiments said disease or condition is cerebral haemorrhage. In some embodiments, said disease or condition is subarachnoid haemorrhage (SAH), such as intracerebral haemorrhage. In some embodiments, said disease or condition is subdural hematoma (SH). In some embodiments, said disease or condition is cerebral infarction. In some embodiments, said disease or condition is cerebral vasospasm. In some embodiments, said disease or condition is spinal injury.

In some embodiments, said disease or condition is non-accidental trauma or paediatric abusive head trauma.

It has also been demonstrated that hypothermia may also be helpful in alleviating injuries caused by drugs overdose (Tan et al. 2020). Thus, in some embodiments, said disease or condition is drug overdose such as the toxicity followed by acetaminophen overdose.

In some embodiments, said disease or condition is recovery after intense exercise.

It has also been demonstrated that hypothermia and scalp cooling is helpful in reducing hair loss in individuals subjected to chemotherapy treatment (Smetanay et al. 2019). Thus, in some embodiments, said disease or condition is hair loss after chemotherapy. It is known that scalp cooling during chemotherapy, for example by means of a cold cap, mar reduce or prevent hair loss due to certain chemotherapeutic drugs, since it narrows the blood vessels beneath the skin of the scalp.

In some embodiments, the composition for use according to the present disclosure is for use in the treatment of a condition for which treatment with mild hypothermia is beneficial, e.g. neonatal asphyxia; stroke; ischemia; cardiac arrest, such as ventricular fibrillation cardiac arrest; migraine; traumatic brain injury; recovery after intense exercise; drug overdose; cerebral haemorrhage; subarachnoid haemorrhage (SAH); subdural hematoma (SH); cerebral infarction; cerebral vasospasm; spinal injury; neonatal abusive head trauma; brachial plexopathy; hair loss after chemotherapy; chronic pain; neuropathic pain; and neurological pain.

In some embodiments, the composition for use according to the present disclosure is for use in the treatment of a condition for which treatment with mild hypothermia is beneficial, preferably: ischemia; migraine; traumatic brain injury; recovery after intense exercise; drug overdose; cerebral haemorrhage; subarachnoid haemorrhage (SAH); subdural hematoma (SH); cerebral infarction; cerebral vasospasm; spinal injury; neonatal asphyxia, such as neonatal abusive head trauma neonatal asphyxia; cardiac arrest, such as ventricular fibrillation cardiac arrest; stroke; hair loss after chemotherapy.

In some embodiments, the composition for use according to the present disclosure is for use in the treatment of a condition for which treatment with mild hypothermia is beneficial, more preferably: neonatal asphyxia; stroke; ischemia; cardiac arrest; migraine; traumatic brain injury; recovery after intense exercise; cerebral haemorrhage; subarachnoid haemorrhage (SAH); subdural hematoma (SH); cerebral infarction; cerebral vasospasm; spinal injury; neonatal abusive head trauma.

In some embodiments, the composition for use according to the present disclosure is for use in the treatment of a condition for which Targeted Temperature Management therapies are beneficial, e.g.: ischemia; headache, such as migraine; traumatic brain injury; recovery after intense exercise; drug overdose; cerebral haemorrhage; subarachnoid haemorrhage (SAH); subdural hematoma (SH); cerebral infarction; cerebral vasospasm; spinal injury; neonatal asphyxia, such as neonatal abusive head trauma neonatal asphyxia; cardiac arrest, such as ventricular fibrillation cardiac arrest; stroke; and hair loss after chemotherapy chronic pain; neuropathic pain; and neurological pain.

In some embodiments, the composition for use according to the present disclosure is for use in the treatment of a condition for which Targeted Temperature Management therapies are beneficial, preferably: neonatal asphyxia; stroke; ischemia; cardiac arrest; migraine; traumatic brain injury; recovery after intense exercise; cerebral haemorrhage; subarachnoid haemorrhage (SAH); subdural hematoma (SH); cerebral infarction; cerebral vasospasm; spinal injury; neonatal abusive head trauma.

In some embodiments, the composition for use according to the present disclosure is for use in the treatment of a condition for which Targeted Temperature Management therapies are beneficial, more preferably: neonatal asphyxia; stroke; ischemia; and cardiac arrest.

In some embodiments, the composition for use according to the present disclosure is for use in the treatment of a condition for which Targeted Temperature Management therapies are beneficial, for example: chronic pain; neuropathic pain; and neurological pain.

In some embodiments, the composition for use according to the present disclosure reduces neuronal death in the individual.

In some embodiments, said composition for use is a substitute for Targeted Temperature Management therapy and/or hypothermia therapy.

One advantage of the present invention is that the benefits of the TTM or hypothermia therapy for an individual are achieved without actually lowering the body temperature of an individual to hypothermic temperatures. Thus, the adverse effects caused by actively cooling a body to mild hypothermic temperatures are reduced or avoided.

Thus, in some embodiments, the Targeted Temperature Management therapy is a therapy wherein the internal core temperature of a subject is lowered between 3° and 36° C., such as between 3° and 35.5° C., between 3° and 35° C., between 30 and 34.5° C., between 3° and 34° C., between 3° and 33.5° C., between 3° and 33° C., between 3° and 32.5° C., between 3° and 32° C., between 3° and 31.5° C., between 30 and 31° C., between 3° and 30.5° C., between 30.5 and 36° C., between 30.5 and 35.5° C., between 30.5 and 35° C., between 30.5 and 34.5° C., between 30.5 and 34° C., between 30.5 and 33.5° C., between 30.5 and 33° C., between 30.5 and 32.5° C., between 30.5 and 32° C., between 30.5 and 31.5° C., between 30.5 and 31° C., between 31 and 36° C., between 31 and 35.5° C., between 31 and 35° C., between 31 and 34.5° C., between 31 and 34° C., between 31 and 33.5° C., between 31 and 33° C., between 31 and 32.5° C., between 31 and 32° C., between 31 and 31.5° C., between 31.5 and 36° C., between 31.5 and 35.5° C., between 31.5 and 35° C., between 31.5 and 34.5° C., between 31.5 and 34° C., between 31.5 and 33.5° C., between 31.5 and 33° C., between 31.5 and 32.5° C., between 31.5 and 32° C., between 32 and 36° C., between 32 and 35.5° C., between 32 and 35° C., between 32 and 34.5° C., between 32 and 34° C., between 32 and 33.5° C., between 32 and 33° C., between 32 and 32.5° C., between 32.5 and 36° C., between 32.5 and 35.5° C., between 32.5 and 35° C., between 32.5 and 34.5° C., between 32.5 and 34° C., between 32.5 and 33.5° C., between 32.5 and 33° C., between 33 and 36° C., between 33 and 35.5° C., between 33 and 35° C., between 33 and 34.5° C., between 33 and 34° C., between 33 and 33.5° C., between 33.5 and 36° C., between 33.5 and 35.5° C., between 33.5 and 35° C., between 33.5 and 34.5° C., between 33.5 and 34° C., between 34 and 36° C., between 34 and 35.5° C., between 34 and 35° C., between 34 and 34.5° C., between 34.5 and 36° C., between 34.5 and 35.5° C., between 34.5 and 35° C., between 35 and 36° C., between 35 and 35.5° C., or between 35.5 and 36° C.

In some embodiments, the Targeted Temperature Management therapy is a therapy wherein the internal core temperature of a subject is lowered between 3° and 36° C., such as between 3° and 35° C., between 3° and 34° C., between 3° and 33° C., between 3° and 32° C., between 3° and 31° C., between 31 and 36° C., between 31 and 35° C., between 31 and 34° C., between 31 and 33° C., between 31 and 32° C., between 32 and 36° C., between 32 and 35° C., between 32 and 34° C., between 32 and 33° C., between 33 and 36° C., between 33 and 35° C., between 33 and 34° C., between 34 and 36° C., between 34 and 35° C., or between 35 and 36° C.

In some embodiments, said composition induces a Mild Cold Response while keeping the body temperature of the individual at 36° C. or higher, such as wherein the body temperature is kept between 36 and 40° C., such as between 36 and 36.5° C., between 36 and 37° C., between 36 and 37.5° C., between 36 and 38° C., between 36 and 38.5° C., between 36 and 39° C., between 36 and 39.5° C.

In some embodiments, said composition induces a mild hypothermia response wherein the individual has a body temperature of 36° C. or higher at the time of administration, such as wherein the individual has a normal body temperature at the time of administration, such as wherein the individual has a body temperature between 36 and 40° C., such as between 36 and 36.5° C., between 36 and 37° C., between 36 and 37.5° C., between 36 and 38° C., between 36 and 38.5° C., between 36 and 39° C., between 36 and 39.5° C. at the time of administration.

In some embodiments, said composition induces a mild hypothermia response wherein the individual has a body temperature of 36° C. or higher after administration of the composition, such as wherein the individual has a normal body temperature after administration of the composition, such as wherein the individual has a body temperature between 36 and 40° C., such as between 36 and 36.5° C., between 36 and 37° C., between 36 and 37.5° C., between 36 and 38° C., between 36 and 38.5° C., between 36 and 39° C., between 36 and 39.5° C. after administration of the composition, such as 1 hour or more after administration of the composition, such as 2 hours or more after administration of the composition, such as 3 hours or more after administration of the composition, such as 5 hours or more after administration of the composition, such as 8 hours or more after administration of the composition.

In some embodiments, the composition further comprises a pharmaceutically acceptable carrier.

### Composition and Compounds

The present disclosure discloses that a compound selected from the group consisting of: a catechol-O-methyltransferase inhibitor, a tyrosine kinase inhibitor, and pharmaceutically acceptable salts thereof, is useful in treatment of a disease or condition as described herein.

Thus, the present disclosure provides that a compound selected from the group consisting of: a catechol-O-methyltransferase inhibitor and a tyrosine kinase inhibitor induces a mild hypothermia response, without actual hypothermia, in an individual in need thereof.

In some embodiments, the compound is selected from the group consisting of: a catechol-O-methyltransferase inhibitor and pharmaceutically acceptable salts thereof.

In some embodiments, the compound is a catechol-O-methyltransferase inhibitor.

Thus, in some embodiments, the compound is a catechol-O-methyltransferase inhibitor selected from the group consisting of Entacapone; Nebicapone; Nitecapone; Opicapone; Tolcapone; and pharmaceutically acceptable salts thereof.

In some embodiments, the compound is a catechol-O-methyltransferase inhibitor selected from the group consisting of Entacapone; Nebicapone; Nitecapone; Opicapone; and Tolcapone.

In some embodiments, the catechol-O-methyltransferase inhibitor is Entacapone or a pharmaceutically acceptable salt thereof.

In some embodiments, the compound is a tyrosine kinase inhibitor or a pharmaceutically acceptable salt thereof.

In some embodiments, the tyrosine kinase inhibitor is Poziotinib or a pharmaceutically acceptable salt thereof.

Thus, in some embodiments, the compound is selected from the group consisting of Entacapone; Nebicapone; Nitecapone; Opicapone; Tolcapone; Poziotinib; and pharmaceutically acceptable salts thereof.

In some embodiments, the compound is selected from the group consisting of: Entacapone; and Poziotinib, or a pharmaceutically acceptable salt thereof.

Several genes e.g., C/RBP/CIRP, RBM3, SP1 consistently show increased expression during mild hypothermia. Thus, increased expression of these genes can be used by the skilled person as a biomarker for induction of the Mild Cold Response.

Thus, in some embodiments, the compound comprised in a composition as described herein increases the transcription of biomarker of the Mild Cold Response.

Thus, in some embodiments, the compound increases transcription of at least one gene selected from the group consisting of: SP1, CIRBP, RBM3.

Thus, in some embodiments, the compound increases transcription of SP1. In some embodiments, the compound increases transcription of CIRBP. In some embodiments, the compound increases transcription of RBM3.

In some embodiments, the compound increases transcription of SP1 and CIRBP. In some embodiments, the compound increases transcription of SP1 and RBM3. In some embodiments, the compound increases transcription of CIRBP and RBM3.

Thus, in some embodiments said composition for use increases transcription of at least one gene selected from the group consisting of: SP1; CIRBP; RBM3, while keeping the body temperature of the individual at 36° C. or higher.

Thus, in some embodiments said composition for use increases transcription of at least one gene selected from the group consisting of: SP1; CIRBP; RBM3, wherein the individual has a body temperature of 36° C. or higher.

Thus, in some embodiments said composition for use inhibits of at least one gene selected from the group consisting of: SMYD5; and BAZ2A, wherein the individual has a body temperature of 36° C. or higher.

In some embodiments, the individual is a mammal. For example, the individual may be a human, a primate, a mouse, a rat, a dog, a cat, a horse.

In some embodiments, the individual has a body temperature between 36 and 40° C., such as between 36 and 36.5° C., between 36 and 37° C., between 36 and 37.5° C., between 36 and 38° C., between 36 and 38.5° C., between 36 and 39° C., between 36 and 39.5° C.

### SMYD5 and BAZ2A Inhibitors

The present inventors have found that a composition comprising an inhibitor selected from the group consisting of: a SMYD5 inhibitor; and a BAZ2A inhibitor, is useful in treatment of a disease or condition as described herein, e.g., a disease or condition which would benefit from induction of a mild hypothermia response.

Thus, in an aspect, the present disclosure concerns a composition for use in the treatment of a disease or condition selected from the group consisting of neonatal asphyxia, stroke, ischemia, cardiac arrest such as ventricular fibrillation cardiac arrest, migraine, traumatic brain injury, recovery after intense exercise, drug overdose, cerebral haemorrhage, subarachnoid haemorrhage (SAH), subdural hematoma (SH), cerebral infarction, cerebral vasospasm, spinal injury, neonatal abusive head trauma, brachial plexopathy, and hair loss after chemotherapy, in an individual in need thereof, the composition comprising an inhibitor selected from the group consisting of: SMYD5 inhibitor and a BAZ2A inhibitor.

Thus, in some embodiments the inhibitor is selected from the group of: a SMYD5 inhibitor; and a BAZ2A inhibitor, preferably a SMYD5 inhibitor.

In some embodiments, the inhibitor is a SMYD5 inhibitor.

In some embodiments, the inhibitor is a BAZ2A inhibitor.

In some embodiments, the inhibitor inhibits SMYD5 and BAZ2A.

SMYD5 and BAZ2A can be inhibited in various way, for example by inhibiting their expression, by blocking their function, or by enhancing their degradation.

Thus, in some embodiments, the inhibitor reduces the expression of SMYD5 in a mammalian cell exposed thereto.

In some embodiments, the inhibitor, such as a SMYD5 inhibitor, induces proteasomal degradation of SMYD5 in a mammalian cell exposed thereto.

In some embodiments, the inhibitor, such as a SMYD5 inhibitor, inhibits the transcriptional activity of SMYD5 in a mammalian cell exposed thereto.

In some embodiments, the inhibitor, such as a SMYD5 inhibitor, inhibits the binding of SMYD5 to the promoter of a gene selected from the group consisting of: SP1 and CIRBP, preferably SP1, in a mammalian cell exposed thereto.

In some embodiments, the inhibitor, such as a SMYD5 inhibitor, inhibits the binding of SMYD5 to the promoter of SP1 in a mammalian cell exposed thereto.

In some embodiments, the inhibitor, such as a SMYD5 inhibitor, inhibits the binding of SMYD5 to the promoter CIRBP in a mammalian cell exposed thereto.

Thus, in an aspect, the present disclosure concerns to a method of inducing a mild hypothermia response in an individual in need thereof, said method comprising inhibiting SMYD5 and/or inhibiting BAZ2A in cells of said individual.

Similarly, in an aspect, the present disclosure concerns to a method of treating a disease or condition selected from the group consisting of neonatal asphyxia, stroke, ischemia, cardiac arrest such as ventricular fibrillation cardiac arrest, migraine, traumatic brain injury, recovery after intense exercise, drug overdose, cerebral haemorrhage, subarachnoid haemorrhage (SAH), subdural hematoma (SH), cerebral infarction, cerebral vasospasm, spinal injury, neonatal abusive head trauma, brachial plexopathy, and hair loss after chemotherapy, in an individual in need thereof, the method comprising administering a pharmaceutically acceptable amount of a SMYD5 inhibitor and/or a BAZ2A inhibitor.

In some embodiments, the method comprises reducing the expression of SMYD5 in a cell of the individual.

In some embodiments, the method comprises promoting proteasomal degradation of SMYD5 in a cell of the individual.

In some embodiments, the method comprises inhibiting the transcriptional activity of SMYD5 in a cell of the individual In some embodiments, the method comprises inhibiting the binding of SMYD5 to the promoter of a gene selected from the group consisting of: SP1 and CIRBP, preferably SP1, in a cell of the individual

In some embodiments, the method comprises:
a) reducing the expression of SMYD5 in a cell of the individual;
b) promoting proteasomal degradation of SMYD5 in a cell of the individual; and/or
c) inhibiting the transcriptional activity of SMYD5 in a cell of the individual; and/or
d) inhibiting the binding of SMYD5 to the promoter of a gene selected from the group consisting of: SP1 and CIRBP, preferably SP1, in a cell of the individual.

Histone modification has previously been found to help integrate effects of cold temperature exposure in diverse organisms. For example, in plants, vernalization, how exposure to cold influences the rate of flowering, is mediated through a Trithorax Polycomb switch (Kim et al., 2009). In some reptiles, sex determination is achieved through a temperature dependent mechanism and for one such, the red eared slider turtle (Trachemys scripta elegans), researchers have recently uncovered the mechanistic basis of this regulation, through a histone methylation switch (Weber et al., 2020). The present inventors have found that SMYD5 is a negative epigenetic regulator of the mammalian mild hypothermia response, and hence the present disclosure provides an example of how histone methylation integrates temperature cues into a fundamental pathway in mammals.

Thus, in some embodiments, the inhibitor, such as the SMYD5 inhibitor, induces tri-methylation at the 4th lysine residue of the histone H3 protein (H3K4me3) and/or inhibits tri-methylation at the 36th lysine residue of the histone H3 protein (H3K36me3) at the promoter of a gene selected from the group consisting of: SP1 and CIRBP, preferably SP1, in a mammalian cell exposed thereto. In some embodiments, the inhibitor, such as the SMYD5 inhibitor inhibits tri-methylation at H3K36me3 at the promoter of a gene selected from the group consisting of: SP1 and CIRBP, preferably SP1, in a mammalian cell exposed thereto.

In some embodiments, the inhibitor, such as the SMYD5 inhibitor induces H3K4me3 at the promoter of a gene selected from the group consisting of: SP1 and CIRBP, preferably SP1, in a cell exposed thereto. In some embodiments, the inhibitor induces H3K4me3 at the promoter of SP1 in a mammalian cell exposed thereto. In some embodiments, the inhibitor induces H3K4me3 at the promoter of CIRBP in a mammalian cell exposed thereto.

In some embodiments, the inhibitor, such as the SMYD5 inhibitor, inhibits H3K36me3 at the promoter of a gene selected from the group consisting of: SP1 and CIRBP, preferably SP1, in a mammalian cell exposed thereto. In some embodiments, the inhibitor inhibits H3K36me3 at the promoter SP1 in a mammalian cell exposed thereto.

In some embodiments, the inhibitor inhibits H3K36me3 at the promoter CIRBP in a cell exposed thereto.

### Indicators of a Mild Hypothermia Response

The present disclosure demonstrates how a newly identified gene, SMYD5, is as a repressor of SP1. Herein evidence is provided, indicating that the inhibition of SMYD5 can be measured, e.g. in terms of SMYD5 mRNA and/or protein expression, and used as a biomarker to identify genetic elements or compounds capable of activating a mild hypothermia response in a mammal.

The inventors also demonstrated that the expression of SMYD5 downstream genes, such as one or more of Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1, is downregulated in presence of mild hypothermia, or an active cold stress response.

Thus, in an aspect, the disclosure is directed to a method for determining whether a genetic element or a compound contributes to a mild hypothermia response in an individual in need thereof, the method comprising the steps of;
a) quantifying the amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in a first sample, wherein said sample is obtained from the individual before administration of the element or compound to said individual;
b) administering the element or compound to said individual;
c) quantifying the amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in a second sample obtained from the same individual as in a), wherein said second sample is obtained after administration of said element; and
d) comparing the amount of a protein quantified a) and b);

wherein a higher or lower amount of protein quantified in c) than in a) is indicative of whether the element contributes to the mild hypothermia response.

Thus, in an aspect, the disclosure is directed to a method for determining whether a element contributes to a mild hypothermia response in an individual in need thereof, wherein the element is selected from the group consisting of: a RNA, such a siRNA, such as a snoRNA; a protein; a post-translational modification such as a histone modification; and a drug, the method comprising the steps of:
a) quantifying the amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in a first sample, wherein said sample is obtained from the individual before the element is administered to the individual;
b) administering the element to the individual;
c) quantifying the amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in a second sample obtained from the same individual as in a), wherein said second sample is obtained after administration of said element; and
d) comparing the amount of a protein quantified in a) and b);

wherein a higher or lower amount of protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in b) than in a) is indicative of whether the element contributes to the mild hypothermia response.

In some embodiments, steps a) and c) of the methods disclosed herein comprise quantifying the amount of proteins encoded by the same one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1.

In some embodiments, steps a) and c) of the methods disclosed herein comprise quantifying the amount of protein encoded by SMYD5.

In some embodiments, steps a) and c) of the methods disclosed herein comprise quantifying the amount of protein encoded by SMYD5 and one or more genes selected from the group consisting of Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1.

In some embodiments, an amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in b) lower than in a) is indicative of the element inducing a mild hypothermia response in the individual.

In some embodiments, an amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in b) higher than in a) is indicative of the element repressing a mild hypothermia response in the individual.

In some embodiments, an amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in b) lower or higher of at least 20%, such as at least 25%, such as at least 30%, such as at least 35%, such as at least 40%, such as at least 45%, such as at least 50% than in a) is indicative of the element contributing to, such as inducing or repressing, a mild hypothermia response in the individual.

In some embodiments, an amount of the protein encoded by SMYD5 in b) lower or higher of at least 20%, such as at least 25%, such as at least 30%, such as at least 35%, such as at least 40%, such as at least 45%, such as at least 50% than in a) is indicative of the element contributing to, such as inducing or repressing, a mild hypothermia response in the individual. In some embodiments, the genetic element is selected from the group consisting of: a RNA, such a siRNA, such as a snoRNA; a protein; a post-translational modification, such as a histone modification.

In some embodiments, the genetic element is a RNA. Thus, in some embodiments, the genetic element is a RNA selected from the group consisting of: siRNA, shRNA, snoRNA. In some embodiment, genetic the element is a siRNA. In some embodiments, the genetic element is a shRNA. In some embodiments, the genetic element is a snoRNA.

In some embodiments, the genetic element is a protein.

In some embodiments, the genetic element is a post-translational modification, such as a histone modification.

In some embodiments, the compound is a drug, for example a small molecule, an antibody, a therapeutic protein.

In another aspect, the present disclosure is directed to a method for monitoring activation of a mild hypothermia response in an individual following administration of a treatment, the method comprising the steps of:
a) quantifying the amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in a first sample, wherein said first sample is obtained from the individual before said treatment;
b) quantifying the amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in a second sample obtained from the same individual as in a), wherein said second sample is obtained after administration of said treatment; and
c) comparing the amount of a protein quantified in a) and b);
d) concluding that the mild hypothermia response is activated by the treatment if the amount of the protein quantified in b) is lower than in a),

wherein a lower amount of protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in b) than in a) is indicative of that the treatment activates the mild hypothermia response.

In some embodiments, steps a) and b) of the methods disclosed herein comprise quantifying the amount of proteins encoded by the same one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1.

In some embodiments, steps a) and b) of the methods disclosed herein comprise quantifying the amount of protein encoded by SMYD5.

In some embodiments, steps a) and b) of the methods disclosed herein comprise quantifying the amount of protein encoded by SMYD5 and one or more genes selected from the group consisting of Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1.

In some embodiments, a lower amount of protein in b) refers to an amount of protein that is at least 10% lower, such as at least 15% lower, such as at least 18% lower, such as at least 20% lower, such as at least 22% lower, such as at least 25% lower than in a), in the disclosed methods.

In some embodiments, a higher amount of protein in b) refers to an amount of protein that is at least 10% higher, such as at least 15% higher, such as at least 18% higher, such as at least 20% higher, such as at least 22% higher, such as at least 25% higher than in a), in the disclosed methods.

As provided herein, the composition for use of the present disclosure, said composition comprising a COMT (catechol-O-methyltransferase) inhibitor or a tyrosine kinase inhibitor, and preferably Entacapone or Poziotinib, induces a mild hypothermia response in the individual to which it is administered.

Thus, in some embodiments, the mild hypothermia response is activated in response to
a. administration of the composition for use disclosed herein; and/or
b. administration of a Targeted Temperature Management (TTM) therapy and/or a hypothermia therapy.

In some embodiments, the amount of protein encoded by one or more of the listed genes is quantified by means of western blot. Similarly, in some embodiments, the gene expression level of one or more of the listed genes is quantified by means of qPCR, such as qRT-PCR.

In some embodiments, the sample is a blood sample, or CSF sample, preferably a blood sample. In some embodiments, the sample is a blood sample.

The present disclosure also relate to of mild hypothermia indicators (MHIs), which allow direct visualization of the promoter activity of genes that respond to mild (32-36° C.) hypothermia.

In an aspect, the present disclosure is directed to a mild hypothermia indicator (MHI) construct comprising:
a) a promoter sequence of a gene selected from the group consisting of SP1, RBM3 and CIRBP; and
b) a nucleic acid sequence encoding a detectable protein, wherein the detectable protein is selected from the group consisting of a fluorescent protein and a luminescent protein.

The MHI constructs of the present disclosure are helpful in detecting and visualizing mild hypothermia or mild hypothermia response in a cell or cell composition through simple fluorescence- or luminescence-based assays.

Preferably, the detectable protein encoded by the MHI construct of the present disclosure is a fluorescent protein. Presence of a fluorophore renders the MHI constructs of the present disclosure suitable for being used in high throughput screening assays, which would not be possible with luminescent proteins. Such MHI constructs comprising a nucleic acid sequence encoding a fluorescent protein are particularly helpful in high throughput screening assays for candidate pharmacological agent for treatment of a disease or condition that normally benefits from mild hypothermia treatment, as defined herein.

In some embodiments, the promoter sequence is of a mammalian gene selected from the group consisting of SP1, RBM3 and CIRBP. For example, the promoter sequence may be of human SP1, RBM3 or CIRBP. The promoter sequence may also be of mouse SP1, RBM3 or CIRBP. The promoter sequence may be also of rat SP1, RBM3 or CIRBP. The promoter sequence may be also of bovine SP1, RBM3 or CIRBP. The promoter sequence may be also of equine SP1, RBM3 or CIRBP. The promoter sequence may be also of porcine SP1, RBM3 or CIRBP. The promoter sequence may be also of primate SP1, RBM3 or CIRBP.

In some embodiments, the MHI construct comprises human or murine SP1 promoter.

In some embodiments, the MHI construct comprises human or murine RBM3 promoter.

In some embodiments, the MHI construct comprises human or murine CIRBP promoter.

In some embodiments, the MHI construct comprises a SP1 promoter of SEQ ID NO: 40 or a functional variant thereof, wherein said variant has at least 80% sequence identity to SEQ ID NO: 40, such as at least 85% sequence identity to SEQ ID NO: 40, such as at least 88% sequence identity to SEQ ID NO: 40, such as at least 90% sequence identity to SEQ ID NO: 40, such as at least 92% sequence identity to SEQ ID NO: 40, such as at least 94% sequence identity to SEQ ID NO: 40, such as at least 96% sequence identity to SEQ ID NO: 40, such as at least 98% sequence identity to SEQ ID NO: 40, such as at least 99% sequence identity to SEQ ID NO: 40.

In some embodiments, the MHI construct comprises a RBM3 promoter of SEQ ID NO: 39 or a functional variant thereof, wherein said variant has at least 80% sequence identity to SEQ ID NO: 39, such as at least 85% sequence identity to SEQ ID NO: 39, such as at least 88% sequence identity to SEQ ID NO: 39, such as at least 90% sequence identity to SEQ ID NO: 39, such as at least 92% sequence identity to SEQ ID NO: 39, such as at least 94% sequence identity to SEQ ID NO: 39, such as at least 96% sequence identity to SEQ ID NO: 39, such as at least 98% sequence identity to SEQ ID NO: 39, such as at least 99% sequence identity to SEQ ID NO: 39.

In some embodiments, the MHI construct comprises a CIRBP promoter of SEQ ID NO: 38 or a functional variant thereof, wherein said variant has at least 80% sequence identity to SEQ ID NO: 38, such as at least 85% sequence identity to SEQ ID NO: 38, such as at least 88% sequence identity to SEQ ID NO: 38, such as at least 90% sequence identity to SEQ ID NO: 38, such as at least 92% sequence identity to SEQ ID NO: 38, such as at least 94% sequence identity to SEQ ID NO: 38, such as at least 96% sequence identity to SEQ ID NO: 38, such as at least 98% sequence identity to SEQ ID NO: 38, such as at least 99% sequence identity to SEQ ID NO: 38.

In some embodiments, the MHI construct further comprises a linker sequence, the linker sequence being positioned between the promoter sequence and the nucleic acid encoding a detectable protein. For example, in some embodiments the MHI comprises a linker, wherein the linker is attached to the 3′ of the promoter sequence and to the 5′ of the nucleic acid sequence encoding a detectable protein. The linker sequence ensures that there is the right distance between the promoter sequence and the nucleic acid encoding the detectable protein.

In some embodiments, the linker sequence is a nucleic acid sequence comprising or consisting of between 2 and 200 nucleotides. In some embodiments, the linker sequence is a nucleic acid sequence, which does not comprise stop codons.

In some embodiments, the linker sequence is a nucleic acid sequence comprising or consisting of any one of SEQ ID NO: 40 to SEQ ID NO: 48, or a functional variant thereof, wherein said variant has at least 80% sequence identity to any one of SEQ ID NO: 40 to SEQ ID NO: 48, such as at least 85% sequence identity to any one of SEQ ID NO: 40 to SEQ ID NO: 48, such as at least 88% sequence identity to any one of SEQ ID NO: 40 to SEQ ID NO: 48, such as at least 90% sequence identity to any one of SEQ ID NO: 40 to SEQ ID NO: 48, such as at least 92% sequence identity to any one of SEQ ID NO: 40 to SEQ ID NO: 48, such as at least 94% sequence identity to any one of SEQ ID NO: 40 to SEQ ID NO: 48, such as at least 96% sequence identity to any one of SEQ ID NO: 40 to SEQ ID NO: 48, such as at least 98% sequence identity to any one of SEQ ID NO: 40 to SEQ ID NO: 48, such as at least 99% sequence identity to any one of SEQ ID NO: 40 to SEQ ID NO: 48.

In some embodiments, the detectable protein is a fluorescent protein. For example, the detectable protein may be a fluorescent protein selected from the group consisting of Green Fluorescent Protein (GFP) and derivatives thereof, and monomeric red fluorescent protein (RFP) and derivatives thereof, and monomeric blue fluorescent protein (BFP) and derivatives thereof.

Examples of derivatives of the GFP are yellow fluorescent protein (YFP), cyan fluorescent protein (CFP), Aequorea coerulescens GFP1 (AcGFP or AcGFP1), enhanced GFP (EGFP), TurboGFP. Many derivatives and variants of GFP are known in the art and can be used in the MHI of the present disclosure. Nucleic acid sequences encoding such detectable proteins are also known in the art and a skilled person is capable of choosing a suitable sequence.

Examples of derivatives of RFPs are mRFP1, mCherry, mStrawberry, mOrange, dTomato.

In some embodiments, the detectable protein is a luminescent protein. For example, the detectable protein may be luciferase.

In some embodiments, the MHI further comprises a response enhancer. For example, the response enhancer may be one or more copies of a Mild Cold Responsive Element (MCRE) sequence.

In some embodiments, the MHI further comprises one or more copies of the MCRE of SEQ ID NO: 50: 5′ TCCCCGCC′3, or a variant thereof, wherein said variant differs from SEQ ID NO: 50 of one nucleotide. For example, the MHI further comprises 1 copy of the MCRE of SEQ ID NO: 50 or a variant thereof, such as between 2 and 20 copies of the MCRE of SEQ ID NO: 50 or a variant thereof, such as between 2 and 19 copies of the MCRE of SEQ ID NO: 50 or a variant thereof, such as between 2 and 18 copies of the MCRE of SEQ ID NO: 50 or a variant thereof, such as between 2 and 17 copies of the MCRE of SEQ ID NO: 50 or a variant thereof, such as between 2 and 16 copies of the MCRE of SEQ ID NO: 50 or a variant thereof, such as between 2 and 15 copies of the MCRE of SEQ ID NO: 50 or a variant thereof, such as between 2 and 14 copies of the MCRE of SEQ ID NO: 50 or a variant thereof, such as between 2 and 13 copies of the MCRE of SEQ ID NO: 50 or a variant thereof, such as between 2 and 12 copies of the MCRE of SEQ ID NO: 50 or a variant thereof, such as between 2 and 11 copies of the MCRE of SEQ ID NO: 50 or a variant thereof, such as between 2 and 10 copies of the MCRE of SEQ ID NO: 50 or a variant thereof, such as between 2 and 9 copies of the MCRE of SEQ ID NO: 50 or a variant thereof, such as between 3 and 9 copies of the MCRE of SEQ ID NO: 50 or a variant thereof, such as between 4 and 9 copies of the MCRE of SEQ ID NO: 50 or a variant thereof, such as between 5 and 9 copies of the MCRE of SEQ ID NO: 50 or a variant thereof, such as between 4 and 8 copies of the MCRE of SEQ ID NO: 50 or a variant thereof, such as 4 copies of the MCRE of SEQ ID NO: 50 or a variant thereof, such as 5 copies of the MCRE of SEQ ID NO: 50 or a variant thereof, such as 6 copies of the MCRE of SEQ ID NO: 50 or a variant thereof, such as 7 copies of the MCRE of SEQ ID NO: 50 or a variant thereof, such as 8 copies of the MCRE of SEQ ID NO: 50 or a variant thereof.

In some embodiments, the response enhancer is positioned at the 5′ of the promoter sequence, for example the 3′ of the response enhancer may be attached to the 5′ of the promoter sequence.

In some embodiments, the response enhancer is positioned at the 3′ of the promoter sequence, for example the 5′ of the response enhancer may be attached to the 3′ of the promoter sequence.

When a response enhancer is present in the MHI of the disclosure, the MHI may further comprise a spacer sequence, said spacer sequence positioned between the response enhancer and the promoter sequence, so that the two elements are at a functional distance, such as are operably linked.

A spacer sequence, when present, may comprise 2 or more nucleotides, preferably 2 to 200 nucleotides, such as a length that a person of skill in the art will deem appropriate, and said spacer sequence will preferably not comprise a stop codon. Elements of an MHI of the present disclosure are operably linked and/or positioned at a functional distance when they are attached to one another, directly or via a linker or spacer sequence, so that they can execute their function, i.e. the promoter sequence is activated in presence of a cold stress response and promotes expression, such as transcription of the nucleic acid encoding a detectable protein; the response enhancer increases transcription of the nucleic acid encoding a detectable protein; the nucleic acid encoding a detectable protein is expressed, such as transcribed.

In some embodiments, the MHI further comprises a selection cassette. The person of skill in the art knows which selection cassettes are available and is capable of selecting a suitable one. The selection cassette may be positioned anywhere in the vector.

For example, a selection cassette suitable for the MHI of the present disclosure is a neomycin selection cassette.

In some embodiments, the MHI construct comprises or consists of a nucleic acid sequence selected from the group consisting of: SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, or a functional variant thereof.

In some embodiments, the functional variant has at least 80% sequence identity to any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33; such at least 82% sequence identity, such at least 85% sequence identity, such at least 88% sequence identity, such at least 90% sequence identity, such at least 93% sequence identity, such at least 95% sequence identity, such at least 98% sequence identity, such at least 99% sequence identity to any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33.

In an aspect, the present disclosure is directed to an expression vector comprising an expression cassette encoding a MHI construct of the present disclosure.

In some embodiments, the expression vector is a plasmid. In some embodiments, the expression vector is a viral vector selected from the group consisting of an adeno-associated viral vector, an adenoviral vector, a lentiviral vector or a retroviral vector. Preferably, in some embodiments, the expression vector is a lentiviral vector.

In some embodiments, the expression vector comprises a selection cassette. A skilled person would know which selection cassettes are suitable for use in the expression vector of the present disclosure.

In some embodiments, the expression vector comprises or consists of a nucleic acid sequence selected from the group consisting of: SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25, or a functional variant thereof.

In some embodiments, the functional variant has at least 80% sequence identity to any one of SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25, such as at least 85% sequence identity, such as at least 88% sequence identity, such as at least 90% sequence identity, such as at least 93% sequence identity, such as at least 95% sequence identity, such as at least 98% sequence identity, such as at least 99% sequence identity to any one of SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25.

In an aspect, the present disclosure is directed to a host cell transformed, transfected or transduced with the expression vector of the present disclosure.

There exists a variety of suitable host cells and cell lines, which may be prokaryotic, e.g., E. coli, or eukaryotic, e.g., CHO cells, COS cells, yeast expression systems, and recombinant baculovirus expression in insect cells. In some embodiment, the host cells or cell line are mammalian cells such as human, mouse, hamster, pig, goat, primate, etc. They may be of a wide variety of tissue types, including mast cells, fibroblasts, oocytes, and lymphocytes, and may be primary cells and cell lines. Specific examples include dendritic cells, U293 cells, HEK293 keratinocytes, peripheral blood leukocytes, bone marrow stem cells and embryonic stem cells.

For example, the host cell may be a cell line suitable for in vitro cell studies, such as a cell line with neuronal characteristics.

In some embodiments, said host cell is a cell obtained from an individual that may benefit from the treatment disclosed herein. Thus, in some embodiment, the host cell may be a cell derived from a blood sample or a skin sample obtained from a mammal, preferably a human.

In an aspect, the present disclosure is directed to an in vitro method for monitoring activation of the mild hypothermia response in a biological sample in response to a treatment, the method comprising the steps of:
a) providing a first biological sample obtained from a mammal before administration of the treatment,
b) contacting the first biological sample with an MHI construct of the present disclosure,
c) determining the level of fluorescence or luminescence in the first biological sample,
d) providing a second biological sample obtained from the same mammal after administration of the treatment,
e) contacting the second biological sample with an MHI construct of the present disclosure,
f) determining the level of fluorescence or luminescence in the second biological sample,

wherein the mild hypothermia response is activated by the treatment if the fluorescence in the second biological sample is higher than in the first biological sample.

In some embodiments, the method comprises contacting a third or further biological sample with an MHI construct of the present disclosure and determining the level of fluorescence or luminescence in said third or further biological sample, so to monitor the activation, progression or regression of a mild hypothermia response.

In some embodiments, the method comprises measuring fluorescence or luminescence at least 15 minutes, such as of at least 20 minutes, such as of at least 30 minutes, such as of at least 45 minutes, such as of at least 60 minutes, such as of at least 2 hours, such as of at least 4 hours, such as of at least 8 hours, such as of at least 12 hours, such as of at least 16 hours after administration of the treatment.

In some embodiments, the method comprises measuring fluorescence or luminescence multiple times after administration of the treatment, for example 1 hour, 2 hours, 4 hours, 8 hours, 12 hours, 16 hours, 20 hours, 24 hours, 28 hours, 32 hours, 36 hours, 40 hours, 48 hours after the start of the exposure, and any time points in between.

In some embodiments, the biological sample is selected from the group consisting of cells and tissues. For example, the biological sample may be a blood sample, a cerebral spinal fluid (CSF) sample.

In an aspect, the present disclosure is directed to an in vitro method for evaluating the effect of a candidate pharmacological agent for treatment of a disease or condition disclosed herein and listed in the section “Therapeutic indications”, the method comprising the steps of:
a) providing mammalian cells transfected with a MHI construct of the present disclosure,
b) measuring fluorescence or luminescence of the cells,
c) contacting the cells with a candidate pharmacological agent,
d) measuring fluorescence or luminescence of the cells after exposure to the candidate pharmacological agent,

wherein a higher fluorescence or luminescence of the cells after exposure to the candidate pharmacological agent is indicative of the agent having an effect for treatment a disease or condition disclosed herein and listed in the section “Therapeutic indications”.

In some embodiments, the mammalian cells are a mammalian cell line.

In some embodiments, the MHI construct comprises a nucleic acid sequence encoding a fluorescent protein, as defined herein.

In some embodiments, the method comprises measuring fluorescence with flow cytometry or with fluorescence microscopy.

In some embodiments, fluorescence or luminescence is measured after an exposure to a pharmacological agent of at least 15 minutes, such as of at least 20 minutes, such as of at least 30 minutes, such as of at least 45 minutes, such as of at least 60 minutes, such as of at least 2 hours, such as of at least 4 hours, such as of at least 8 hours, such as of at least 12 hours, such as of at least 16 hours.

In some embodiments, fluorescence or luminescence is measured multiple times during and/or after an exposure to a pharmacological agent, for example 1 hour, 2 hours, 4 hours, 8 hours, 12 hours, 16 hours, 20 hours, 24 hours, 28 hours, 32 hours, 36 hours, 40 hours, 48 hours after the start of the exposure, and any time points in between.

In some embodiments, an at least 2-fold higher, such as at least 2.25-fold higher, such as at least 2.5-fold higher, such as at least 2.75-fold higher, such as at least 3-fold higher, such as at least 3.25-fold higher, such as at least 3.5-fold higher, such as at least 3.75-fold higher fluorescence or luminescence of the cells after exposure to the candidate pharmacological agent is indicative of the agent having an effect for treatment a disease or condition disclosed herein and listed in the section “Therapeutic indications”.

In some embodiments, fluorescence is measured by means known to the person of skill in the art. For example, fluorescence may be measured by using flow cytometry, e.g. FACS, or fluorescent microscopy.

In some embodiments, luminescence is measured by means known to the person of skill in the art. For example, luminescence may be measured by using a luminometer.

These methods describing a method for monitoring activation the mild hypothermia response in an individual following a treatment, can be used for example to determine activation of the mild hypothermia during treatment of any of the diseases or conditions described herein.

Currently in the clinic, mild hypothermia is induced by cooling an individual by means of cooling catheters, cooling blankets, application of ice applied around the body of the individual, and other methods. It takes several hours to reach the goal temperatures, depending also on the weight of the individual, and there are so far no other measurements than the body temperature, which can be used to determine if the wanted response, a mild hypothermia response as defined under “Definitions” herein, has been achieved.

The present disclosure provides a method that can be used to assess and determine if a mild hypothermia response as defined under “Definitions” herein, has been achieved, as well as to monitor its state. This method can be used both to assess and monitor currently used methods of inducing mild hypothermia and to assess and monitor a mild hypothermia response induced according to the present disclosure, i.e. by administering to an individual in need thereof the composition for use provided herein.

### EXAMPLES

### Example 1: Material and methods

### Creation of Novel Indicators

PGL4.10 plasmid (Promega, #9PIE665) was used as the backbone for the disclosed CIRBP and SP1 indicators, where the luc2 sequence was cut from the plasmid with NcoI (NEB) and XbaI (NEB).

The promoter sequence for CIRBP was cloned from mouse sequence using CIRBP forward primer 5′tcgataggtaccTGGCTTCACAAATGCGCCTCAGT3′ (SEQ ID NO: 2) and CIRBP reverse primer 3′cctaaggcagatctGCGAGGGGGAGCGCAAGAGT5′ (SEQ ID NO: 3). Restriction enzymes KpnI (NEB) and BglII (NEB) were used to insert the promoter into the plasmid.

The promoter sequence for SP1 was cloned from human DNA using the forward primer 5′tcaagtcaggctagcGCAACTTAGTCTCACACGCCTTGG3′ (SEQ ID NO: 4) and reverse primer 3′cagtgctgcctcgagGCTCAAGGGGGTCCTGTCCGG5′ (SEQ ID NO: 5).

Restriction enzymes NheI (NEB) and XhoI (NEB) were used to insert the promoter into the plasmid.

AcGFP1 was cloned from PT7XbG2-AcGFP1 (Novopro, #V002843) vector using the reverse primer 3′cggcggagTCTAGAATTACTTGTACAGCTCGTCC5′ (SEQ ID NO: 6) and the forward primer 5′taagccaccATGGTGAGCAAGGGCGAGGAGC3′ (SEQ ID NO: 7). Restriction enzymes NheI (NEB) and XhoI (NEB) were used to insert the GFP into the plasmid.

The neomycin selection cassette was cloned from pROSA26-dest plasmid (Addgene, #21189) using: the forward primer 5′CATTATCGTCGACTCTACCGGGTAGGGGAGGCGCTT3′ (SEQ ID NO: 8) and reverse primer 3′CGCCGCCGACGATAGTCAAGCTTCTGATGGAATTAGAACTTGGC5′ (SEQ ID NO: 9). Restriction enzymes SalI (NEB) and PshAI (NEB) were used to insert the neomycin cassette into the plasmid.

CIRBP and SP1 indicators were made with and without Mild Cold Responsive Element (MCRE) (Sumitomo et al., 2012). The MCRE sequence 5′ TCCCCGCC′3 (SEQ ID NO: 50) was inserted in 5 repeats in front of the promoter sequences with a linker sequence between. Acc651 (NEB) restriction enzyme was used to insert the MCRE sequence into the CIRBP indicator and Acc651 (NEB) and SacI (NEB) restriction enzymes were used to insert the MCRE into the SP1 indicator.

Sanger sequencing was used to validate insertion sites, size, and sequence of insertion.

The RBM3 promoter with MCRE enhancer (6 repeats—Table 1) in front of the promoter was cloned into the multiple cloning site pCMV6-AC-GFP (BlueHeron, #PS100010). Lentiviral vectors were made also for CIRP, SP1 and RBM3 on a pLV-Ex (VectorBuilder) lentiviral vector. The CIRBP promoter (Table 1) for the lentiviral vector was designed from the human promoter and the SP1 promoter was moved upstream of the transcription start site (TSS) but RBM3 promoter sequence remained unchanged.

Included were MCRE (8 repeats for CIRBP lentiviral indicator 5 repeats for other), promoter, neomycin selection cassette and mCherry fluorescent protein behind each promoter sequence.

The promoter region, size and backbone plasmid for each indicator is detailed in Table 1. Some variation was also observed in absolute levels among individual indicators/experiments, but the pattern was always consistent. Thus, only data from individual experiments are provided in any figure.

TABLE 1
specifics of the MHIs, the selection marker used was Neomycin.

| | Promoter | | | | | |
|---|---|---|---|---|---|---|
| | sequence, | | | | | |
| | location | | | Repeats | | |
| | in bp from | | | of | Fluorescent | Backbone |
| | TSS | Animal | Location | MCRE | protein | plasmid |
| CIRP | −452 to +72 | Mouse | Chr. 10: | 5 | AcGFP | pGL4.10 |
| Indicator | | | 80167244- | | | |
| | | | 80168059 | | | |
| RBM3 | −667 to +229 | Human | Chr. X: | 6 | TurboGFP | pCMV6- |
| Indicator | | | 48573772- | | | AC-GFP |
| | | | 48574678 | | | |
| SP1 | −281 to −20 | Human | Chr. 12: | 5 | AcGFP | pGL4.10 |
| Indicator | | | 53379972- | | | |
| | | | 53380272 | | | |
| CIRP | −448 to +153 | Human | Chr. 19: | 8 | mCherry | pLV-Ex |
| lentiviral | | | 1268820- | | | |
| Indicator | | | 1269420 | | | |
| RBM3 | −667 to +229 | Human | Chr. X: | 5 | mCherry | pLV-Ex |
| lentiviral | | | 48573772- | | | |
| indicator | | | 48574678 | | | |
| SP1 | −358 to −96 | Human | Chr. 12: | 5 | mCherry | pLV-Ex |
| lentiviral | | | 53379895- | | | |
| Indicator | | | 53380156 | | | |

### Culture of Cell Lines

HEK293 (herein also referred to as 293) cells were cultured in DMEM/F12 with GlutaMAX (Gibco, cat. no. 10565018) with 10% filtered FBS (Gibco, cat. no. 10270106). Cells were cultured at 37° C. unless otherwise stated, then 37° C. served as a control.

Transfection of Indicators into Cell Lines

HEK293 cell lines were transfected when 70-90% confluency was reached. Either Lipofectamine™ 2000 or 3000 (Thermofisher Scientific, #11668027, #L3000015) was used according to the manufacture's protocol. The fluorescence of the indicators was measured via FACS up to 48 hours after transfection. FACS data were analysed with the FlowJo Software, where gates were created on living cells, single cells, GFP positive cells and the fluorescence for that population was measured.

### Drug Screen and Validation

Briefly, Lipofectamine 3000 (Thermofisher Scientific, L3000015) was used to transfect the SP1+MCRE indicator into the HEK293FT cell line. 24 h after transfection, 30000 cells were plated into each well of a 96 well black well plate. 48 h after transfection the cells were exposed to FDA Approved Drug Screening Library (L1300-Z298012) via the High Throughput Screening Services (ChemCore) at Johns Hopkins University. Each drug was exposed in 20 μM concentration. Total well GFP was read with the ClarioStarPlus Plate Reader (settings: gain 1500, 470-15 excitation and emission 515-20 at focal height 2.6) 16, 28, and 40 h after exposure of the library. Increase in fluorescence was calculated with dividing GFP reads from positive control at each time point with GFP reads of each well exposed to drug at the same timepoint for wells in the same lane. Compounds were validated in same concentration (20 μM). For validation cells were exposed to the drug for 24 hours, and evaluated by FACS as described in the section Transfection of indicators into cell lines.

Making of HEK293WT Cell Lines that Stably Express Cas9 and Cas9 Plus SP1 Indicator

The present inventors made two different stable cell lines from HEK293WT cells, one that expressed Cas9 (lentiCas9-blast, Addgene #52962), referred to as 293WT+Cas9, and other that expressed both Cas9 (lentiCas9-blast, Addgene #52962) and the SP1 MHI described herein, referred to as 293WT+SP1+Cas9. These cell lines were made by transfecting the plasmids with lipofectamine and selecting stably transfected cells using Blastocidin selection for Cas9 and Neomycin selection to select for SP1 MHI.

Genome Wide CRISPR-Cas9 Screen on Fluorescent HEK293WT Cell Lines that Stably Express Either SP1 MHI or RBM3 Lentiviral MHI

Using the 293WT+Cas9+SP1 and 293WT+Cas9 cell lines the present inventors performed a GeCKO screen, where the sgRNA pool made from library A and B (Addgene, #1000000049) was transduced in MOI 0.3 (Shalem et al. 2014; Sanjana et al. 2014; Joung et al. 2017). The RBM3 lentiviral indicator, MOI 1, and the GeCKO sgRNA library were transduced simultaneously (referred to 293WT+Cas9+RBM3). The Joung et. al. protocol was used with a few exceptions, e.g. a few more cycles of PCR in cases in which material was insufficient. For making of the lentivirus, we used a protocol described by Kutner et. al. Lentiviral sgRNA library concentration was done with Amicon ultracentrifugal filters (Millipore, #UFC9003). RBM3 lentiviral MHI was concentrated using PEG, the 4× Lentivirus concentrator solution protocol from the MD Anderson Cancer center at University of Texas (available online) was used. 16 hours after the transduction of the sgRNA library, cells were selected with puromycin for 6 days and 8 hours before the cells were moved to 32° C. for 16 hours. Lowest puromycin selection concentration was decided based on a killing curve for 293WT cells. Following 7 days of puromycin selection, the cells were harvested and sorted with FACS. The 5% highest fluorescent cells for 293WT+Cas9+SP1 and 293WT+Cas9+RBM3 and 5% lowest fluorescent cells for 293WT+Cas9+SP1 were sorted with the Cell sorter SH800Z from Sony. Next, gDNA was isolated and PCR was performed for the samples as previously described (Shalem et al. 2014; Sanjana et al. 2014; Joung et al. 2017). For the 293WT+Cas9+SP1 a two step PCR was performed, but for the 293WT+Cas9+RBM3 a one step PCR was performed, as previously described (Shalem et al. 2014; Sanjana et al. 2014; Joung et al. 2017). Next generation sequencing (NGS), single read with 80 cycles and 8 indexing cycles with PhiX spike in of 20%, was performed for HEK293WT+Cas9+SP1 with NovaSeq S4 at deCODE genetics and NovaSeq PE150 at Novogene for the HEK293WT+Cas9+RBM3; coverage was >500 reads/sgRNA. According to cell number after puromycin selection, the coverage was >700 times for HEK293WT+Cas9+SP1 and >850 times coverage for HEK293WT+Cas9+RBM3 of the sgRNA library. The NGS libraries were sequenced with NovaSeq 6000 S4; the NGS libraries from HEK293WT+Cas9+SP1 had a range of 32-280 million reads per sample, and NovaSeq PE150 the NGS libraries from HEK293WT+Cas9+RBM3 which ranged 51-88 million reads per sample.

### Real Time Quantitative Polymerase Chain Reaction (RT-qPCR)

Total RNA from cells incubated at 32° C. or 37° C. was isolated, using Direct-zol™ RNA Microprep (Zymo Research, R2062), according to the manufacturer's instructions. The concentration of RNA was measured by NanoDrop™ One Microvolume UV-Vis Spectrophotometer (Thermo Scientificv™) followed by cDNA synthesis with High-Capacity cDNA Reverse Transcription Kit (Applied Biosystems™, 4368814) on MiniAmp™ Thermal Cycler (Applied Biosystems™). qPCR was performed using Luna® Universal qPCR Master Mix on CFX384™ Real-Time PCR Detection System (Bio-Rad). Each biological replicate of the RT-qPCR assay in this study was carried out in technical triplicates. Any technical replicate that deviated from other replicates by 0.4 cycle threshold (Ct) was removed from calculations of average Ct values. The primers used in this study are as follows:

SP1 (fwd):
(SEQ ID NO: 10)
5′-CACCCAATTCAAGGCCTGCCGT-3′;
SP1 (rev):
(SEQ ID NO: 11)
5′-GGGTTGGGCATCTGGGCTGTTT-3′;
RBM3 (fwd):
(SEQ ID NO: 12)
5′-GAGACTCAGCGGTCCAGGGGTT-3′;
RBM3 (rev):
(SEQ ID NO: 13)
5′-CCTCTGGTTCCCCGAGCAGACT-3′;
CIRBP (fwd):
(SEQ ID NO: 14)
5′-CCGAGTTGACCAGGCTGGCAAG-3′;
CIRBP (rev):
(SEQ ID NO: 15)
5′-TCCATAGCCCCGGTCTCCTCCT-3′;
GAPDH (fwd):
(SEQ ID NO: 16)
5′-TCAAGGCTGAGAACGGGAAG-3′;
GAPDH (rev):
(SEQ ID NO: 17)
5′-CGCCCCACTTGATTTTGGAG-3′.
SMYD5 1 (fwd):
(SEQ ID NO: 34)
5′-GCACTGTGCGCAAAGACCTCCA-3′
SMYD5 2 (fwd):
(SEQ ID NO: 35)
5′-GGAAACCAGGCCAGGTTCTGCC-3′
SMYD5 1 (rev):
(SEQ ID NO: 36)
5′-CTGGGCACAGGACCTGGTGGTA-3′
SMYD5 2 (rev):
(SEQ ID NO: 37)
5′-GGCTGCCAACCGACATTCTGCA-3′

### Western Blot Assay

Cells were washed with PBS and then lysed for 30 min on ice in RIPA buffer (50 mM Tris HCl ph8, 150 mM NaCl, 1% NP-40, 1% Sodium deoxycholate, 0.1% SDS, 2 mM EDTA, phosphatase inhibitor (Cell signaling, Cat. #5870S), and Halt™ Protease Inhibitor Cocktail (Thermo Fisher, Cat. #78437). The lysates were centrifuged at 16,000×g for 20 minutes at 4° C. and the supernatants were collected. The supernatants were diluted with 4× loading buffer (Li-cor) and heated at 95° C. for 5 mins. The protein concentration was measured using Pierce™ BCA Assay Kit (Thermo Fisher). 20-30 μg protein samples were loaded into gels and separated by SDS-PAGE. They were transferred to polyvinylidene fluoride (PVDF) membranes and blocked in 5% bovine serum albumin in Tris-buffered saline with Tween 20 for 1 hour. Primary antibodies (rabbit anti-CIRBP, 1:2000, Cat. #10209-2-AP, Proteintech; rabbit anti-RBM3, 1:1000, CAT. #14363-1-AP, Proteintech; rabbit anti-SP1 1:1000, CAT. #21962-1-AP, Proteintech; mouse anti-Lamin 1, 1:5000, CAT. #66095-1-Ig, Proteintech; rabbit anti-SMYD5, 1:1000, in-house; rabbit anti-Phospho-SP1, 1:1000, CAT. #PA5-104771, Proteintech) were applied to respective membranes after washing, and incubated at 4° C. overnight. Membranes were incubated with IRDye® secondary antibodies (anti-rabbit 800, cat. #926-32213; anti-mouse 680, cat #926-68072, missing manufacturer) at room temperature for 90 mins. After washing, the protein bands were visualized on the Odyssey® CLx Infrared Imaging System and quantified by Image J software.

### DNA Studies and Analysis

HEK293 cells (in triplicates) were split into two batches and put into 37° C. and 32° C. After 6 hours, cells were harvested and DNA isolated with a Zymo Quick-DNA™ Midiprep Plus Kit (Zymo Research, CA, D4075). Samples were submitted to the Genetic Resources Core Facility (Johns Hopkins University), bisulfite treated, and run on the Infinium MethylationEPIC BeadChip Kit (Illumina, California, US) with two technical controls (50% and 100% DNAm, 8 samples total). Technical controls were created using mixtures of 100% and 0% methylated HCT116 DKO (D50414-1/2, Zymo Research, Irvine, CA, USA). DNA methylation data (IDAT files) containing unmethylated and methylated intensity values were imported to R 4.2.1 for analysis (R core team 2022). Raw data was processed using single sample approach with preprocessNoob( ) function and methylation values (beta values) were obtained using the getBeta( ) function from minfi package version 1.42.0 (Fortin et al 2017; Triche et al. 2013; Aryee et al. 2014). The model matrix was created for the two temperatures using the model.matrix( ) function from stats R package and sva( ) function from sva package version 3.44.0 was applied with two estimated surrogate variables (R core team 2022; Leek J T, et al. 2022). The model was fit with contrast matrix using ImFit( ), with the estimated coefficients and standard errors generated using contrasts.fit( ) from limma package version 3.52.4 (R core team 2022; Ritchie et al. 2015). The p-values were moderated using eBayes( ) function and CpG cites ranked according to their p-value (Ritchie et al. 2015).

### Cut&Run

HEK293 were isolated and Cut&Run performed according to Epicypher CUTANA protocol (v1.7) on 300.000 cells per sample. Cells were permeabilized with 0.01% Digitonin and for normalization, the present inventors spiked in E. coli DNA (Epicypher, 23618-1401) at the final concentration of 0.2 ng per sample. The library preparation was performed with TrueSeq-Chip Sample preparation Kit (Illumina, cat. no. #IP-202-9001). The following antibodies were used for the Cut&Run: Anti-Flag-antibody (Sigma, cat. no. F1804), CTCF antibody (Cell Signaling, cat. no. 2899S), H3K4me3 (EpiCypher, cat. no. 13-0041), H3K9me3 (Abcam, cat. no. ab176916), H3K27me3 (Thermofisher, cat. no. MA5-11198), H3K36me3 (Thermofisher, cat. no. MA5-24687), H4K20me3 (EpiCypher, cat. no. 13-0054) and Rabbit IgG Antibody (Epicypher, cat. no. 23613-0042).

### Apoptosis Assay

Apoptosis assay was based on that published by Xiang et al.2016. 293WT+Cas9+SP1 cell line was exposed either to 1-5 μM H₂O₂ or vehicle (H₂O) for 4 hours at 32° C. or 37° C. 4 h after exposure gMFI of the cells were analyzed with FACS as described in the section Transfection of indicators into cell lines.

### Data Analysis

For the analysis of fluorescence in the drug screen and FACS data, Excel from Microsoft, GraphPad Prism (v.9) and FlowJo (v.10.8.1) were used. All statistical testing was done with GraphPad Prism (v.9).

### GeCKO Analysis

To analyze the GeCKO screen data, single read fastq files for each replicate and condition were merged using cat command. Then, MaGeCK (v.0.5.9) was used to identify enriched sgRNA's in sorted samples (Li et al. 2017) and sgRNA counts were normalized to control for sgRNA's. R studio was used to visualize the results and to filter genes. To select for interesting genes, a list of genes was exported that had both FDR value under 0.25 and LFC value over 2.5 for each condition in both screens. Then, genes were excluded, which were present in both SP1 High versus Control and SP1 Low versus Control lists as they most likely promote cell growth and therefore were present in both lists. The same genes were excluded from the RBM3 High versus Control list for the same reasons.

### Cut&Run Analysis

We followed published STAR protocols. Briefly, first we trimmed the raw Fastq reads in paired mode with TrimGalore (v.0.6.7) then we used Bowtie2 (v.2.4.4.) (Langmead et al. 2012) to align the reads to Hg38 and E. coli reference genome, both downloaded from UCSC. Overall alignment to Hg38 were from 83.6-98.63% and to E. coli spike in were from 0.1-4.18%. The Sam files were then converted to Bam files, indexed, and sorted via SAMtools (v.1.15) (Li et al. 2009) and BedGraphtoBigWig (v.4) from BEDTools (v.2.3) (Quinlan and Hall 2010).

### Example 2: Mild Hypothermia Indicators Show Increased Fluorescence at Mild Hypothermia (32° C.)

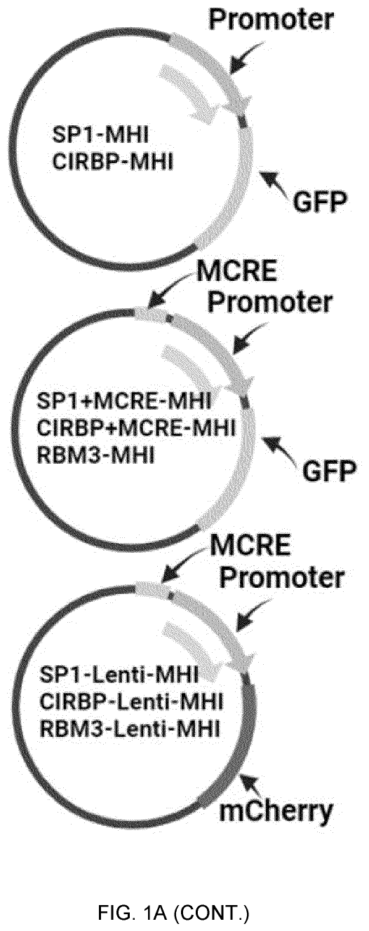

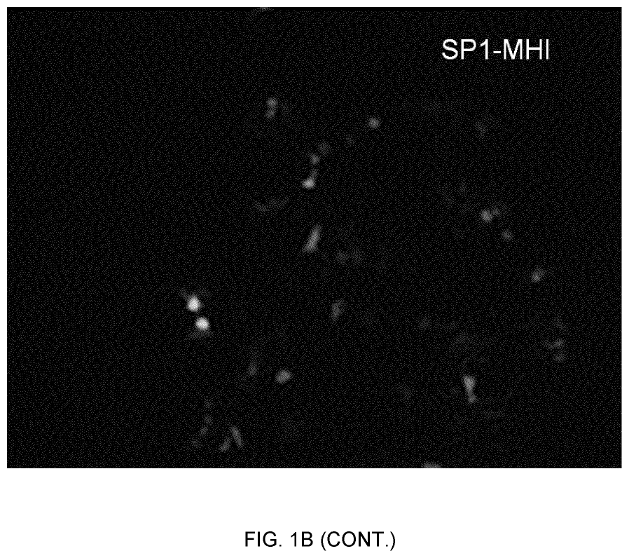

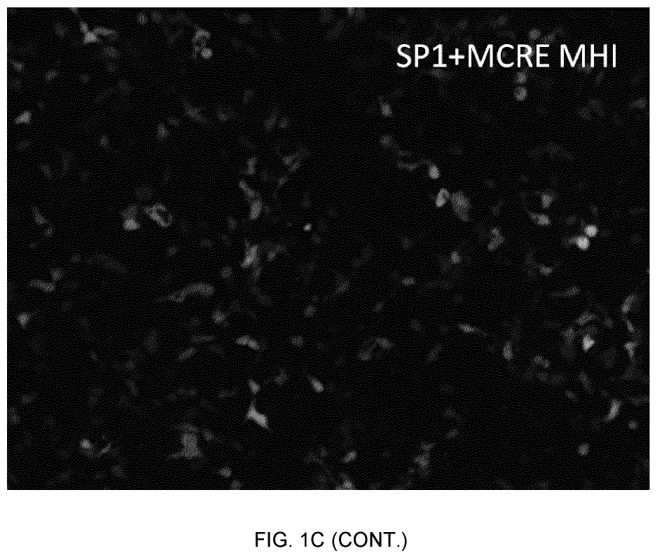

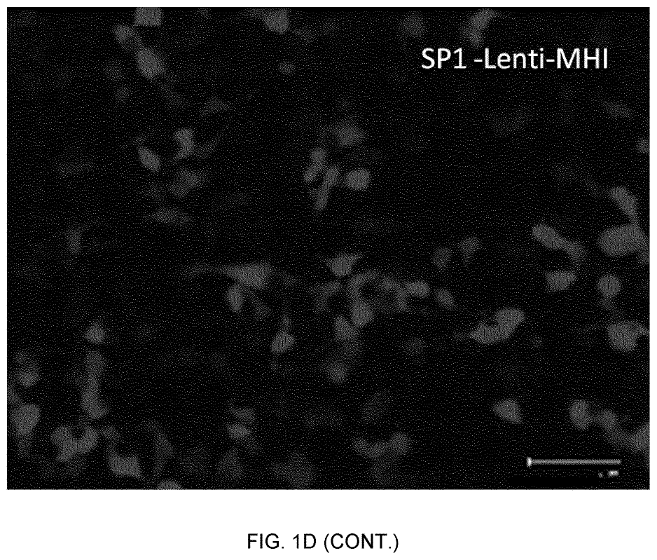

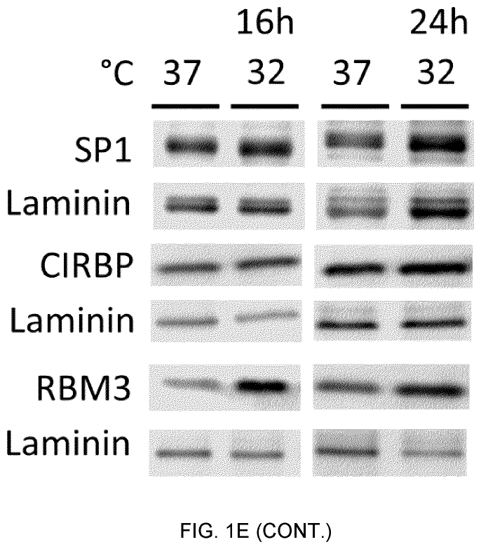

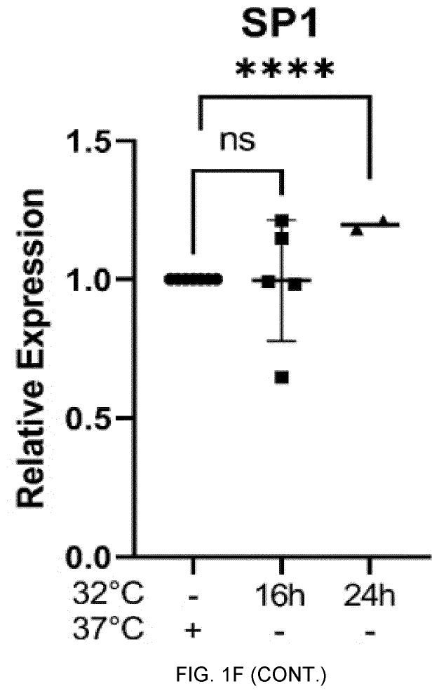

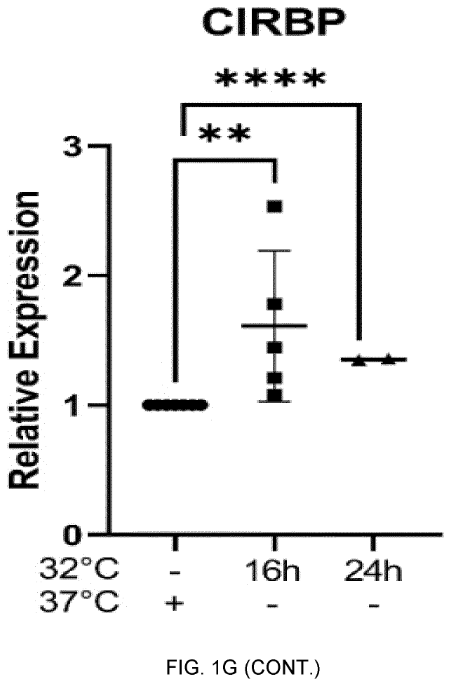

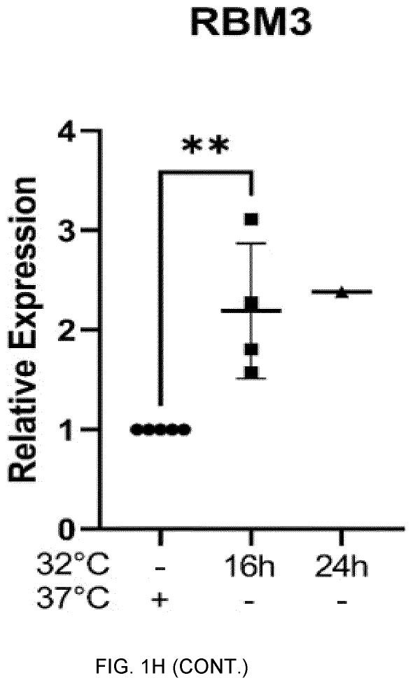

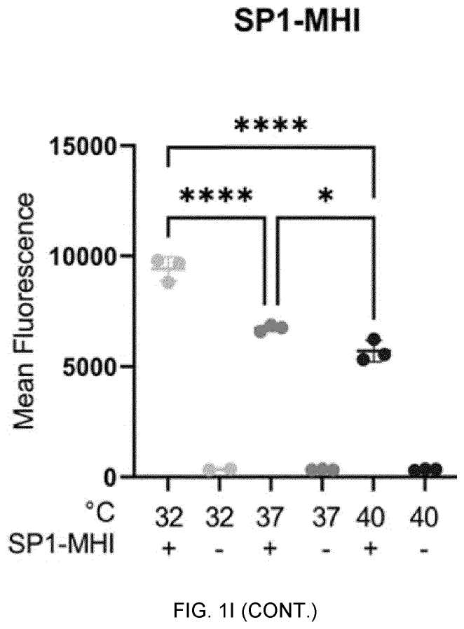

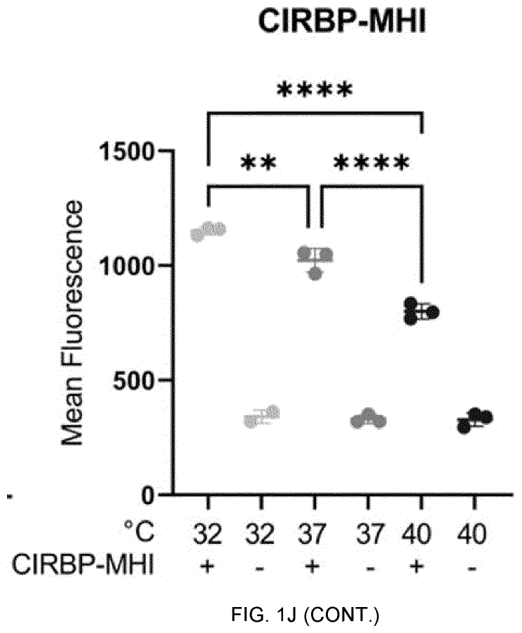

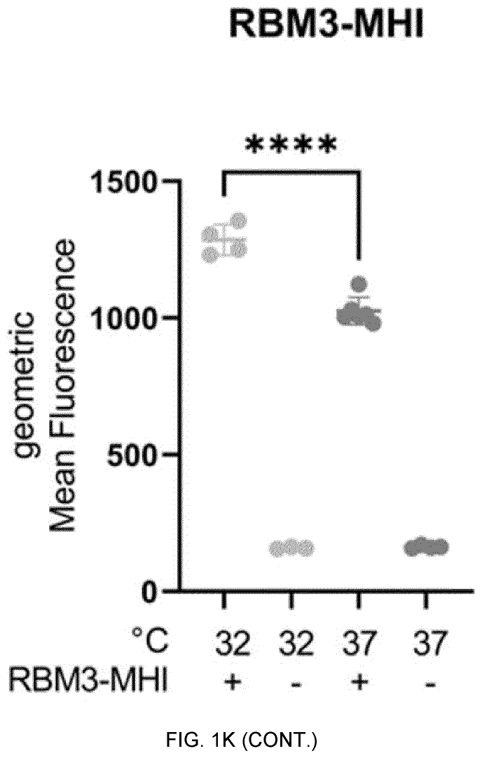

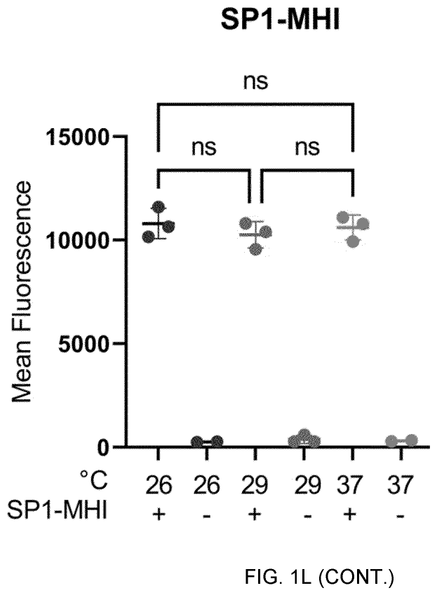

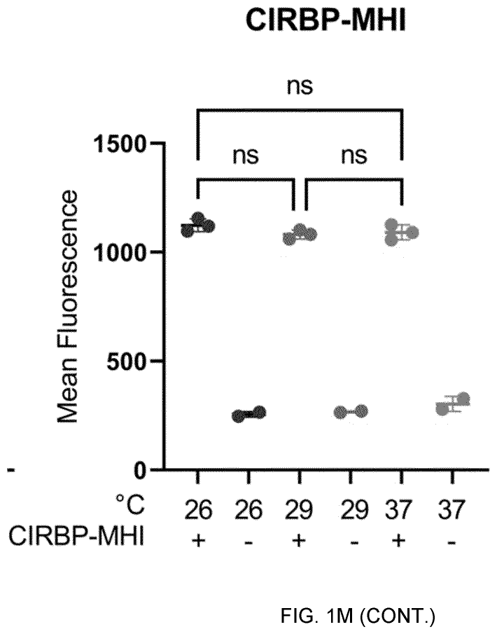

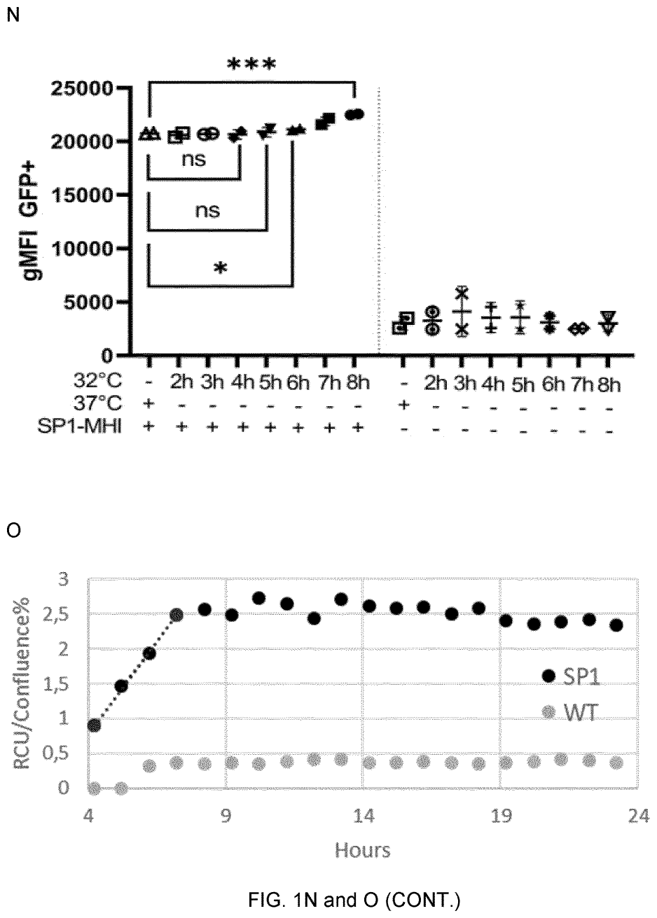

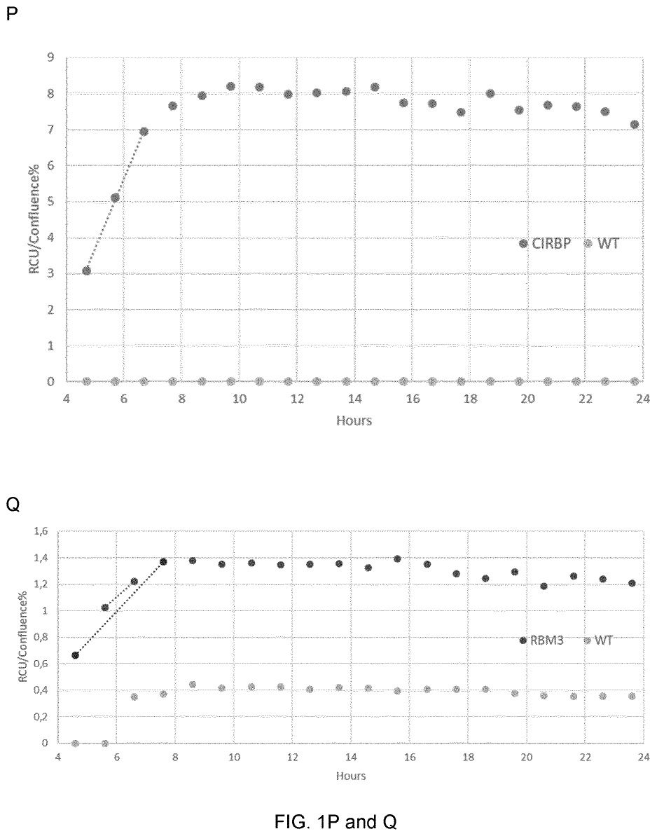

The presently disclosed array of mild hypothermia indicators (MHIs) allows direct visualization of the promoter activity of the three best known genes shown to respond to mild (32-36° C.) hypothermia (CIRBP, RBM3, SP1, FIG. 1A, critical range indicated with bold text).

The present inventors developed eight distinct MHIs (Table 1) all of which comprise a promoter with or without Mild Cold Response Element (MCRE) sequence, followed by a sequence encoding for GFP or mCherry.

All MHIs demonstrated increased fluorescence at 32° C. compared to 37° C., as visualized by a fluorescent microscope with relatively low fluorescence at 37° C. (FIG. 1B-1D and FIG. 1I-M). To show that the generated cell system (HEK293) is responsive to 32° C. compared to 37° C., a significant increase of all three endogenous proteins by Western blot at 16 or 24 hours was demonstrated (FIG. 1E-H). Indicators were transiently transfected into HEK293 cells at five different temperatures (26° C., 29° C., 32° C., 37° C. and 40° C., FIG. 1L-M) for 16 h followed by fluorescence-activated cell sorting (FACS). Indicators representing all three genes (SP1, CIRBP, RBM3) had significantly (P<0.0001) increased fluorescence (quantified by FACS) at 32° C. compared to 37° C./40° C. (FIG. 1I-1M) and the response was uniformly strongest at 32° C. (FIG. 1N). At the same time, MHIs did not show increased activity at lower (26° C., 29° C.) or higher (40° C.) temperatures, supporting the hypothesis that the developed MHIs specifically capture the mild hypothermia response and are not activated by general cell stress (i.e., heat stress or moderate hypothermia). Given the single cell resolution of MHIs, the temporal dynamics of promoter activity of the three indicators was assessed. Using FACS, maximal increase in fluorescence of SP1-MHI was observed between 6-8 h (P<0.05, FIG. 1D). We validated this with a separate method (continuous monitoring of fluorescence from IncucyteS3), again observing maximal intensity within 6-8 h, with SP1-MHI, rising fastest, followed by RBM3-MHI, and then CIRBP-MHI (FIG. 1O-1QG).

### Conclusion:

The MHIs of the present disclosure are responsive to mild hypothermia and not to general cell stress such as heat stress or moderate hypothermia.

### Example 3: Compound Screening Suggests Candidates that Activate Mild Hypothermia Response in an Apoptosis and Cell Cycle Independent Manner

The present inventors hypothesized that if the mild hypothermia response had a number of regulators upstream of the three genes currently known (SP1, CIRBP, RBM3), it might be possible to chemically dissect said response with drugs, which in addition to providing insights into the response, could identify potential therapeutic targets for Targeted Temperature Management (TTM).

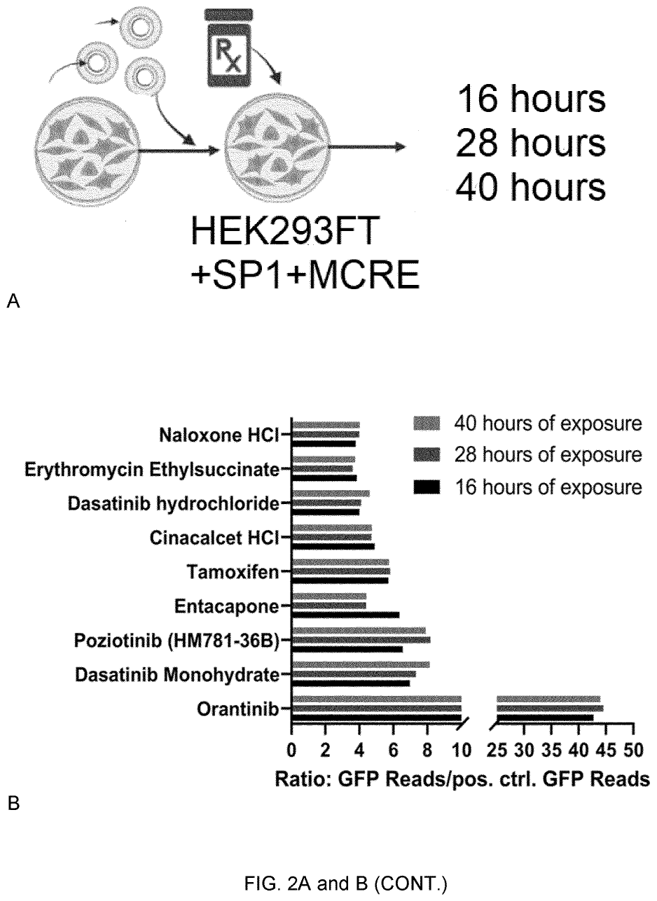

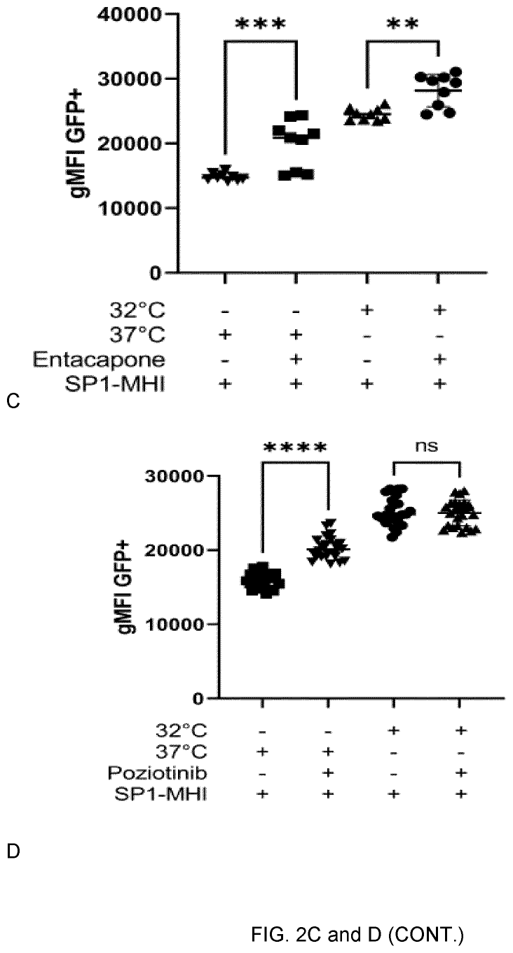

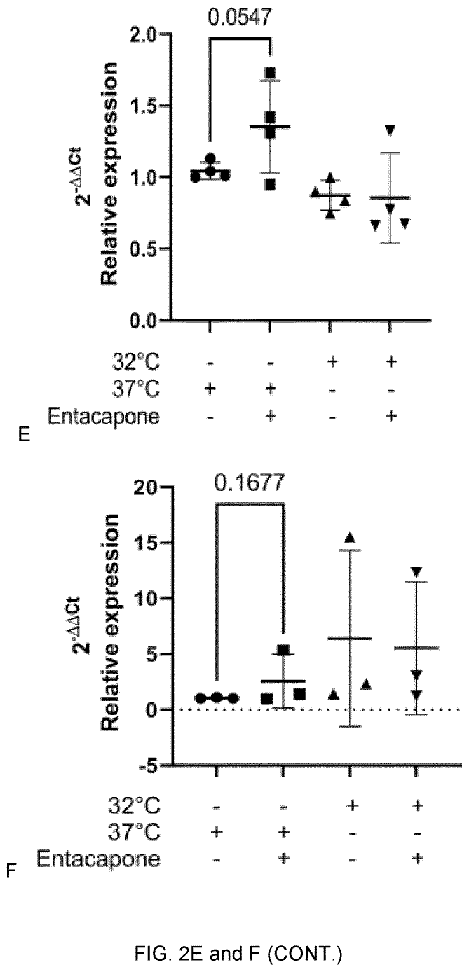

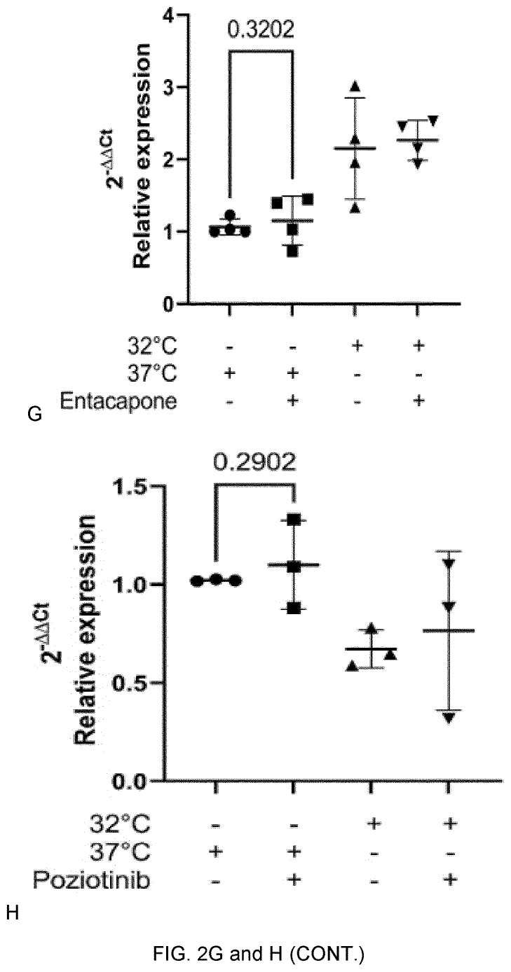

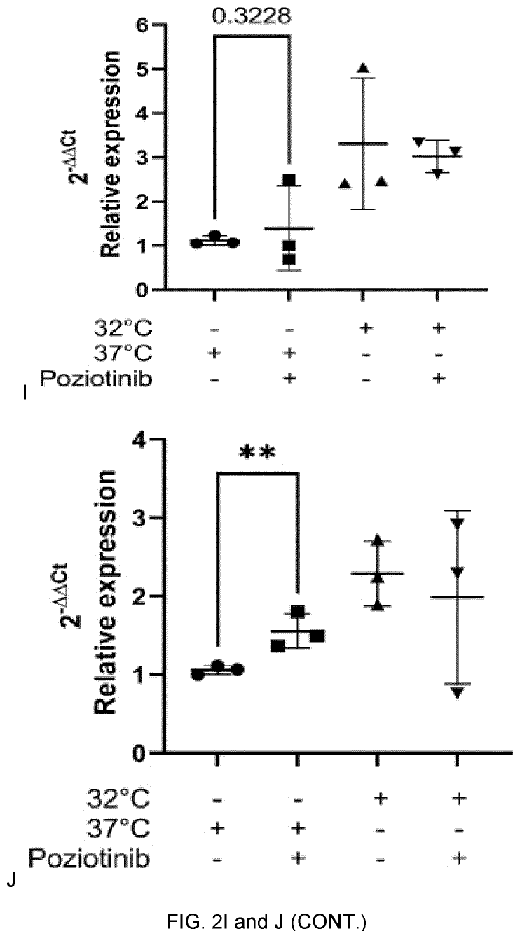

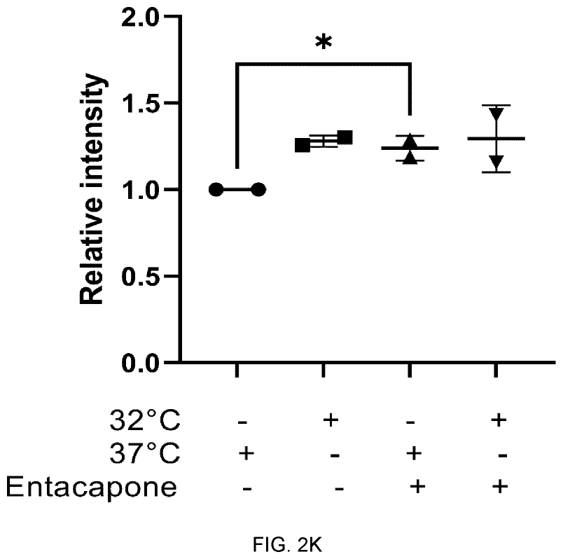

To explore this in an unbiased manner, a systematic library drug screen (1953 FDA-approved inhibitors) was performed using HEK293FT cells transfected with the SP1+MCRE-MHI. As this indicator gave the strongest total intensity, it is particularly useful for compound screening. Cells transfected with said MHI were exposed to the drug library (20 μM concentration) and fluorescence was measured after 16-, 28- and 40-hours of drug exposure. Drugs that could increase the indicator's fluorescence at least >3.75 times at one of the time points were further investigated. Among these genes, it was noted an enrichment of Tyrosine Kinase Inhibitors including Orantinib, Dasatinib, Poziotinib, Rociletinib, Afatinib, and Lapatinib. Three from this group were validated (Dasatinib, Orantinib, and Pozitinib) and another 5 that affect unrelated pathways were selected (Cincalcet, Entacapone, Erythromycin, Naloxone, and Tamoxifen, FIG. 2B). Tolcapone (COMPT inhibitor) caused relative 1.8×, 1.7×, 1.9× increase of fluorescence of the SP1+MCRE-MHI after 16 h, 28 h, 40 h exposure when compared to control (SP1+MCRE-MHI cells without drug exposure). (data not shown).

HEK293 cells with Cas9 and SP1-MHI were individually exposed to each compound (20 μM) for 16 hours at either 32° C. or 37° C. followed by FACS. Two compounds, showed significantly (P<0.001, P<0.0001) increased fluorescence (FIG. 2C-D) at 37° C. (Entacapone, Poziotinib), indicating activation of response with drug without hypothermic stimulus. One drug (Entacapone) could significantly (P<0.01) potentiate fluorescence levels at 32° C.

To exclude the possibility of cell cycle arrest or increased apoptosis, cell cycle phase of HEK293 cells exposed to Pozitinib and Entacapone was evaluated. Any signs of cell cycle arrest in HEK293 was observed for either compound but after 16 hours exposure of Entacapone at both 32° C. and 37° C., a slight increase in percentage of total cells in the S phase of cell cycle was observed. Similarly, there were no signs of activation of the MHIs after exposure to an apoptotic agent.

To evaluate effects on the endogenous loci (CIRBP, RBM3 and SP1) real time quantitative polymerase chain reaction (RT-qPCR) was carried out after 16 h of either 32° C. or 37° C.—with or without the exposure to the compound during that time.

Unlike MHIs, which all contain a stable GFP and thus have a long half-life, it is possible that mRNA dynamics are significantly faster given the importance of cells rapidly adjusting to temperature changes. Supporting that thought, only RBM3 expression was significantly (P<0.05) increased after 16 h of Poziotinib exposure at 37° C. (FIG. 2J) but all showed a trend towards increase (FIG. 2H-2J). Western blotting after 24 h exposure at either 32° C. or 37° C., with or without exposure to the compounds during that same time showed a significant difference (P<0.05, FIG. 2K) for Entacapone, suggesting that the Western blot might be more sensitive to differences than RT-qPCR.

### Conclusion:

The MHIs of the present disclosure were successfully used to identify agents that induce mild hypothermia in and in vitro cell assay.

### Example 4: CRISPR-Cas9 Knockout Screens with SP1 or RBM3-MHIs Reveal Candidate Regulators

In an effort to map regulators of the cooling response (SP1, RBM3) in an unbiased manner, the available lentiGuide-Puro pooled sgRNA library (Shalem et al. 2014; Sanjana et al. 2014; Joung et al. 2017) was used. These screens were performed on SP1-MHI (SEQ ID NO: 21) and RBM3-lenti-MHI (SEQ ID NO: 24) as these reflect the earliest currently known start points of the mild hypothermia response.

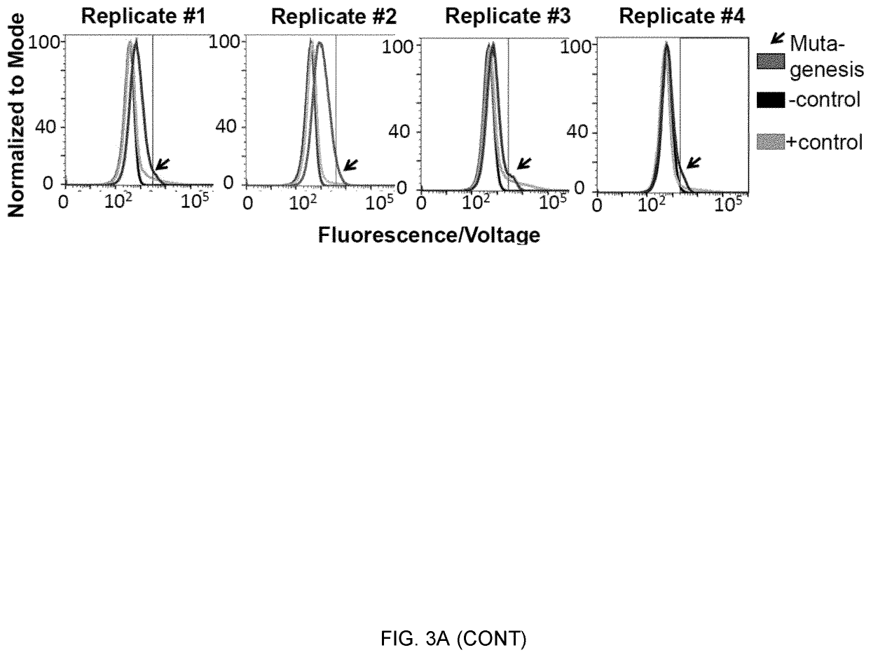

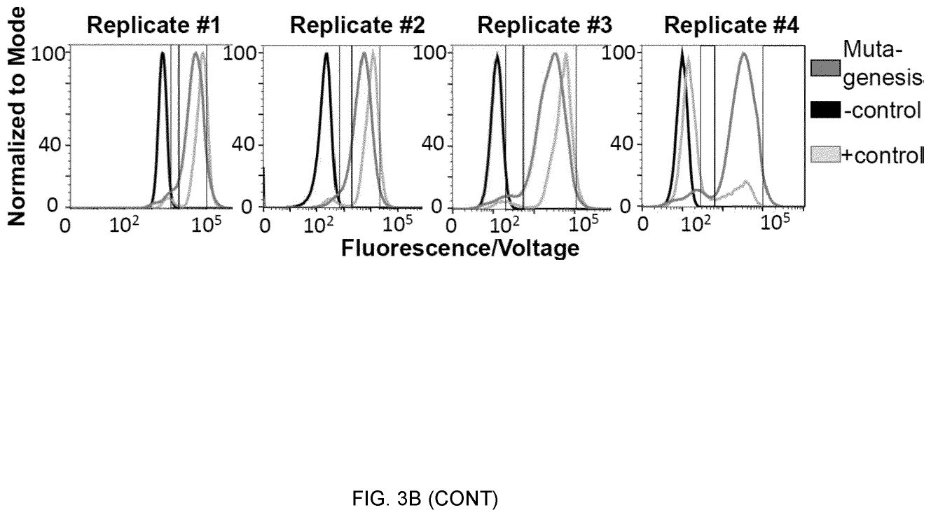

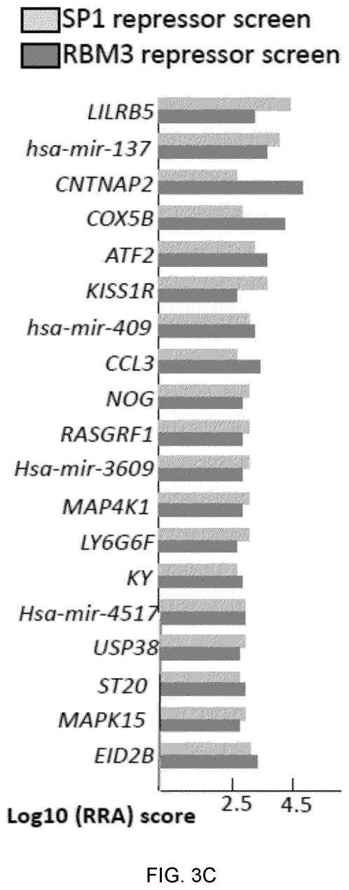

The screen was performed on HEK293WT+Cas9 cells containing SPI-MHI, fluorescently tracking SP1 promoter activity. After puromycin selection and exposure to hypothermia for 16 hours (to activate said MHI), cells were harvested followed by sorting of the 5% most and least fluorescent cells to capture repressors and activators of SP1, respectively. After exposure to the sgRNA library in four biological replicates (FIG. 3A), a consistent shift towards lower fluorescence was observed for the population of cell transfected with SP1-MHI compared to the positive control (non-transduced HEK293WT+Cas9+SP1-MHI). Seeing such a dramatic shift in fluorescence suggests a large number of genes are upstream of SP1, and may suggest that that the majority are activators rather than repressors for the SP1 arm of the mild hypothermia response.

RBM3-MHI had a tendency to be silenced over time, therefore, to shorten passage time HEK293WT+Cas9 were transduced with a lentiviral mCherry based RBM3-MHI (RBM3-lenti-MHI) and the sgRNA GeCKO library simultaneously. An obvious shift to increased fluorescence was observed—capturing RBM3 promoter activity for 3 of the 4 replicates, although this was most evident for the first 2 replicates (FIG. 3B). By the same reasoning, this may indicate that there are many genes upstream of RBM3, but potentially more repressors than activators.

Since the curves for negative control cells and HEK293+Cas9+RBM3 transduced cells were overlapping on left side (candidate activators), only the 5% most fluorescent cells were collected (candidate repressors).

For both screens guides were amplified and deep next generation sequencing was performed. To identify guides enriched in sorted populations, the inventors used the MaGeCK pipeline (Li et al. 2014). Since there were few factors with very strong enrichment, the inventors decided to cast a wide net and look at all genes that had a −Log 10(RRA score)>2.5 in an effort to not miss possible regulators. 21 genes were excluded, as they were seen in both lists for the high and low sorts of SP1, as these may be related to proliferation rather than the mild hypothermia response itself.

A list of 621 genes and micro-RNAs for RBM3-repressors, 495 genes for the SP1-repressors, and 61 for SP1-activators remained—a clear overrepresentation of repressors to activators. The two screens only shared 19 candidate repressors (FIG. 3C) which is no different than expected by chance, given the size of the lists. Although, noteworthy shared repressors included two MAP kinase activators (MAP4K1 and MAPK15) and a micro-RNA known to target SP1 (hsa-mir-137). The limited overlap suggests that SP1 and RBM3 may be regulated independently, or alternatively that there are few shared genes or that our screen was not extensive enough to yield shared regulators.

### Conclusion:

The MHIs of the present disclosure were successfully used to identify cellular regulators of mild hypothermia response.

### Example 5: Pathway Analysis Points to Potential Negative Regulation by the Epigenetic System

The present inventors did not observe shared pathways amongst the 15 top ranked MSigDB pathways for SP1-activators, SP1-repressors or RBM3 repressors. However, it was noted that histone methylation showed up as a significantly enriched pathway for RBM3 repressors but not for the other two groups. Histone modification (including histone methylation) is a dynamic gene regulatory system, known to dictate temperature-dependent sex determination in reptiles and vernalization in plants. Using an established list of epigenetic machinery factors (www.epigeneticmachinery.org/), the present inventors found no highly ranked candidate SP1 activators but did find 12 epigenetic machinery (EM) genes as highly ranked candidate RBM3 repressors (KAT8, BAZ2A, SFMBT2, C14orf169, KAT6A, PRDM7, CHD4, SETD3, CDYL2, MBD6, CHD7, and KDM4D) and 3 genes to be highly ranked candidate SP1 repressors (SMYD5, KAT5, HDAC6). All 15 genes had Log 10(RRA) scores above 2.5. For such a gene to have a direct primary involvement in SP1 or RBM3 regulation as a repressor, it must either place a repressive mark or remove an active mark. Five genes (BAZ2A, SFMBT2, C14orf169, HDAC6 and SMYD5) had the potential of being direct repressors and only two of them were among top 100 hits in either screen: BAZ2A, which has a role in modulating DNA methylation (DNAm) and SMYD5, a histone methyltransferase, which is known to place H3K20 and H3K36 trimethylation.

### Conclusion:

The MHIs of the present disclosure were successfully used to identify epigenetic regulators of mild hypothermia response.

### Example 6: SMYD5 Validates as a Repressor of SP1

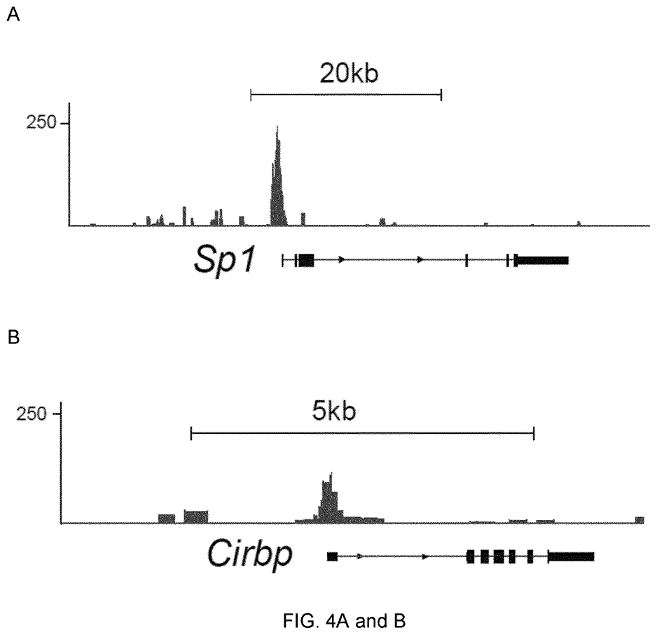

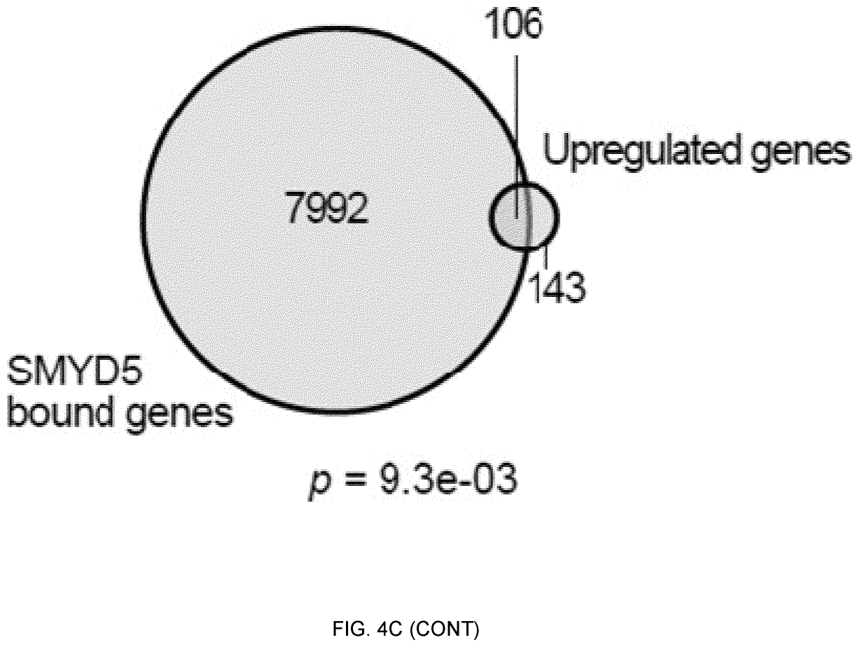

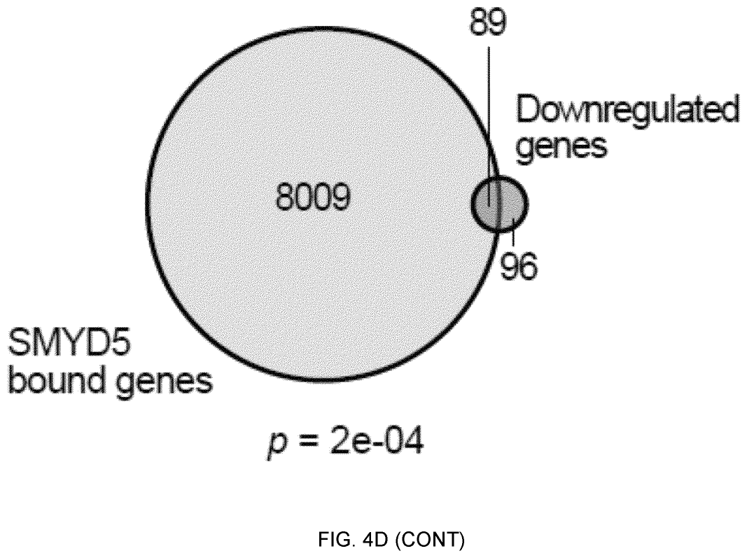

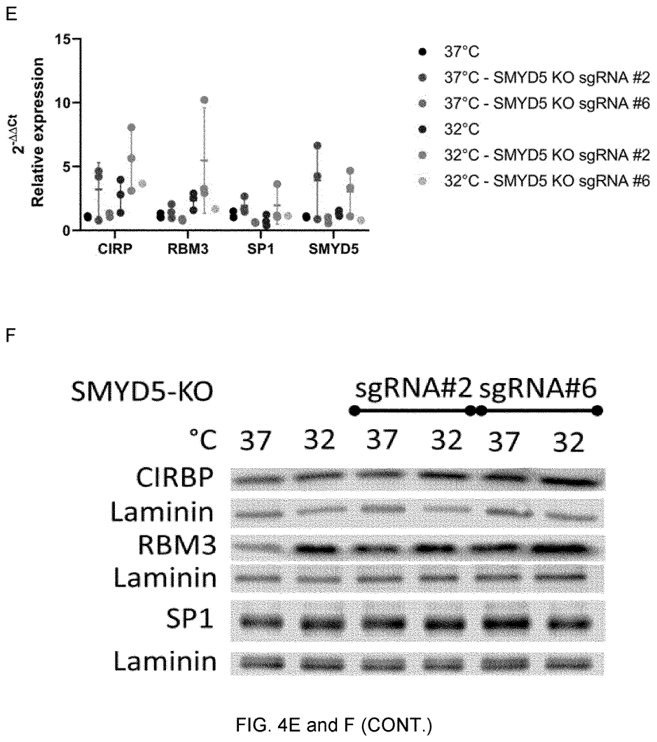

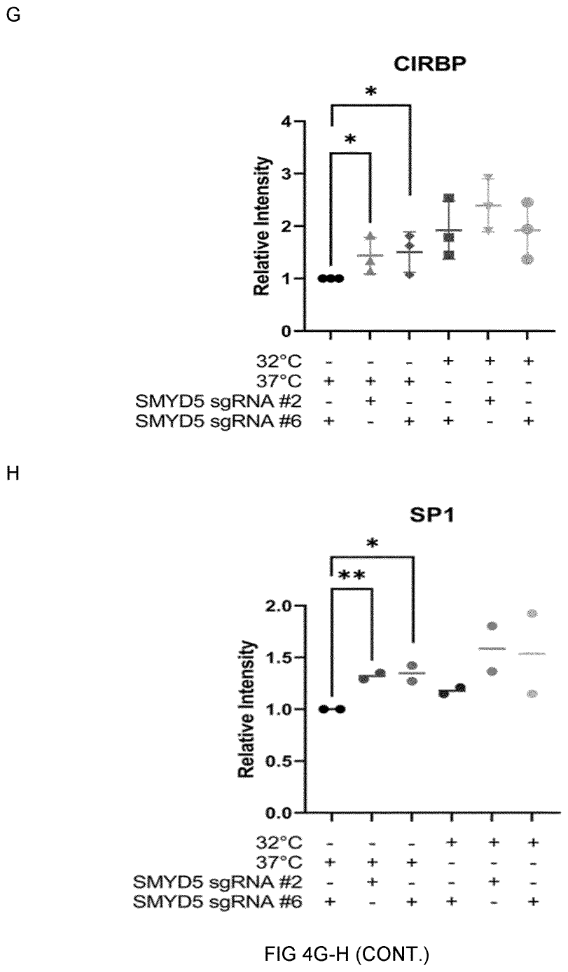

To explore SMYD5 further, the present inventors took advantage of a newly published dataset (Zhang et al. 2022) which performed Cut&Run assay in mouse Embryonic Stem Cells (mES) with an overexpressed SMYD5-Flag. Upon re-analyzation of the data, it was noticed that SMYD5, has strong peaks at the promoters of both SP1 (130K) and CIRBP (70K), but not RBM3 (FIG. 4A-B). These peaks both occur at evolutionarily conserved sequences and overlap CpG islands. Taking advantage of a RNA-Seq dataset on SMYD5-knockout (SMYD5-KO) mESC from same group (Zhang et al. 2022) yielded approximately equal amounts of upregulated and down-regulated genes among SMYD5 bound loci (FIG. 4C-D) supporting prior reports that SMYD5 can place both open and closed chromatin marks (H3K36me3, and H4K20me3) (Zhang et al. 2022; Kidder et al. 2017). Further, the re-analyzed data showed that SMYD5 knockout yielded increased expression of SP1 but not CIRBP or RBM3 supporting a role for SMYD5 as a direct SP1 repressor in a murine system. After KO of SMYD5 in HEK293 cells a trend was observed towards increased expression of SP1 comparing WT and SMYD5-KO with qRT-PCR. Furthermore, a significant increase of SP1 and CIRBP, but not RBM3, was observed following SMYD5-KO at 37° C. (FIG. 4E-I).

### Conclusion:

Knockout of SMYD5 in HEK293 cells resulted in increased expression of SP1.

### Example 7: SMYD5 Shows Temperature Limited Expression In Vitro and In Vivo

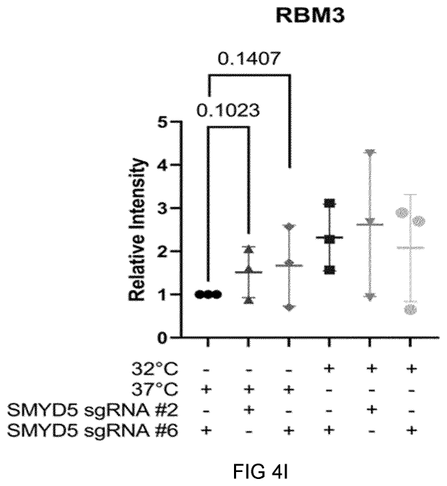

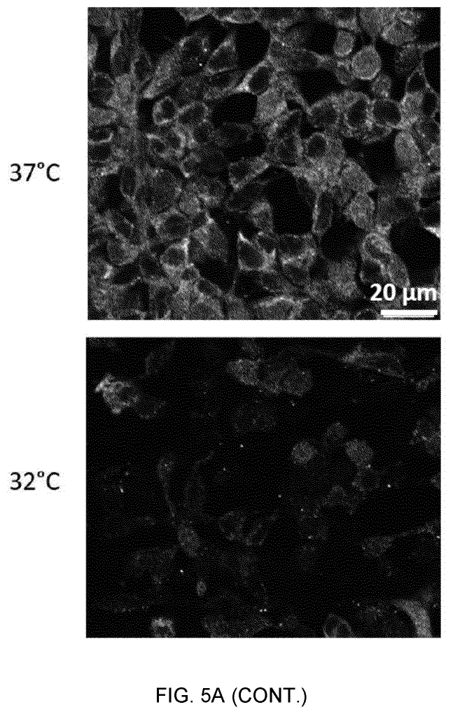

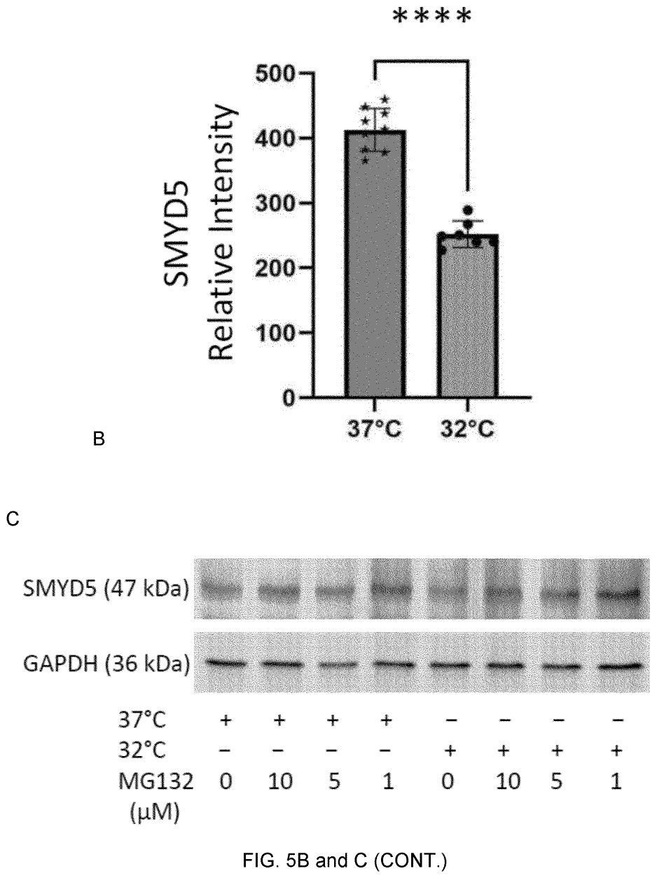

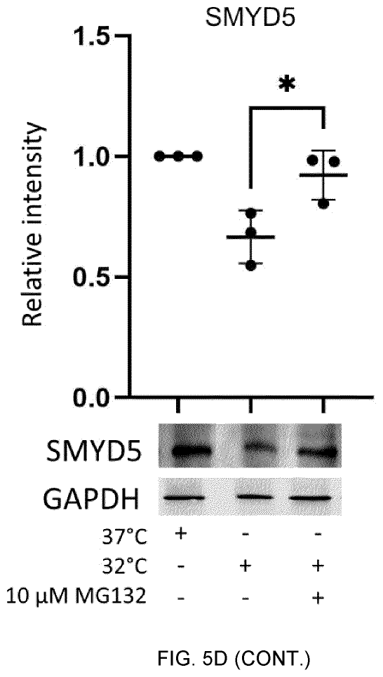

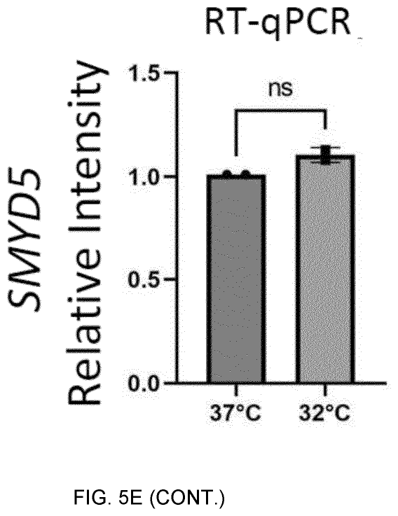

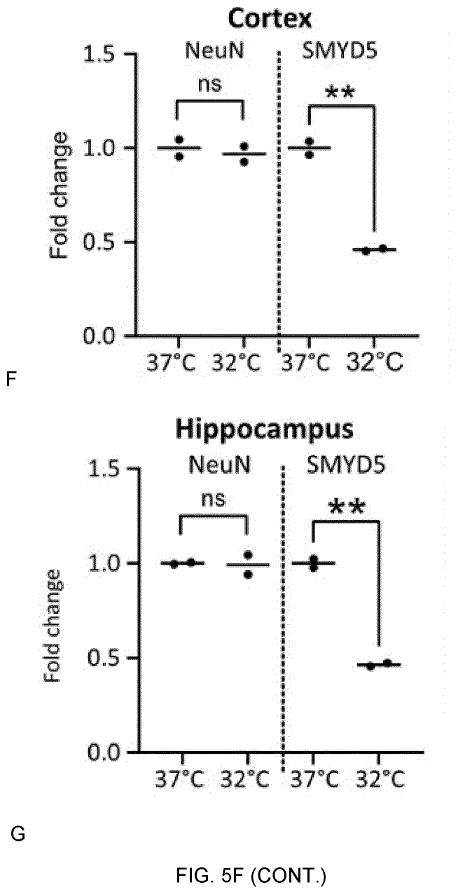

To test whether SMYD5 shows differential expression at 37° C. and 32° C., we performed immunofluorescence staining using a custom antibody against SMYD5 (Aljazi, et al. 2022). We observed nuclear and cytoplasmic staining at 37° C. (FIG. 5A). The expression of SMYD5 was significantly decreased at 32° C. compared to 37° C. (FIG. 5A). We performed this experiment multiple times and characterized the intensity of the two signals (SMYD5, DAPI) and observed a statistically significant difference of SMYD5 intensity normalized to DAPI comparing the two temperatures (FIG. 5B). We also validated using an anti-FLAG antibody in cells with overexpression of SMYD5-FLAG, which yielded a similar result. We validated further by performing a Western blot from HEK293 at 32° C. and 37° C., showing significant more staining at 37° C. than 32° C. (FIG. 5C). This difference was not noted at the transcriptional level (FIGS. 5D and E) indicating that SMYD5, is primarily regulated post-transcriptionally. As SMYD5 has previously been found to have direct binding of UBA52 (Huttlin et al. 2021), a supplier of ubiquitin and three other ubiquitin related factors (USP34, PARK2, TRIM25), and a regulator of the ribosomal protein complex stability of SMYD5 was explored, the proteasome was inhibited and loss of temperature dependence was observed at 32° C. (FIG. 5D) indicating that SMYD5 is degraded as an early step of the mild hypothermia cascade. In mice treated with a standard hypothermia regimen we also observed temperature dependence of SMYD5 in vivo at two distinct neuronal populations (Cortex and Hippocampus, FIG. 5F).

### Conclusion:

SMYD5 was degraded as an early step of the mild hypothermia cascade at 32° C.

### Example 8: Epigenetic and Expression Consequences of Differing SMYD5 Levels at 37° C. Versus 32° C.

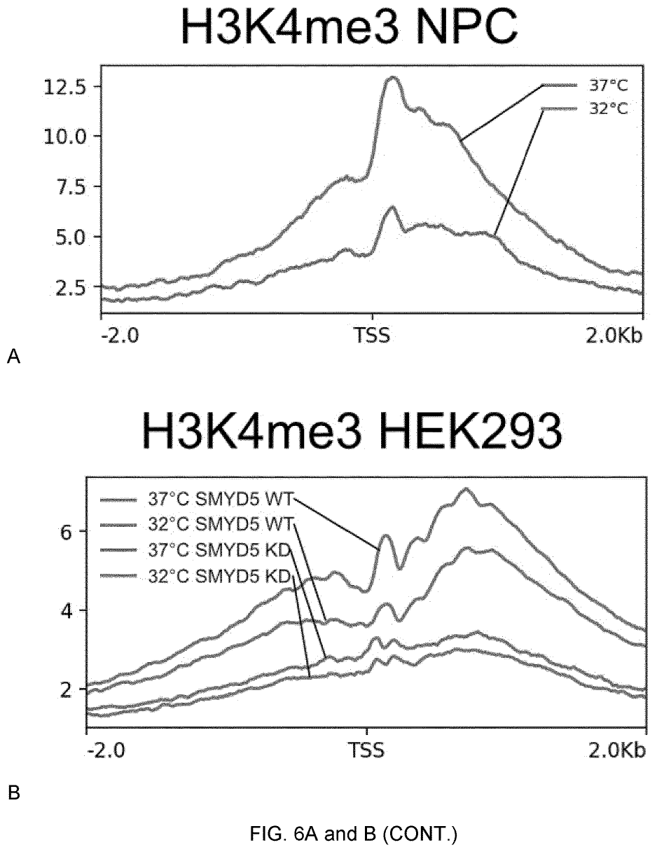

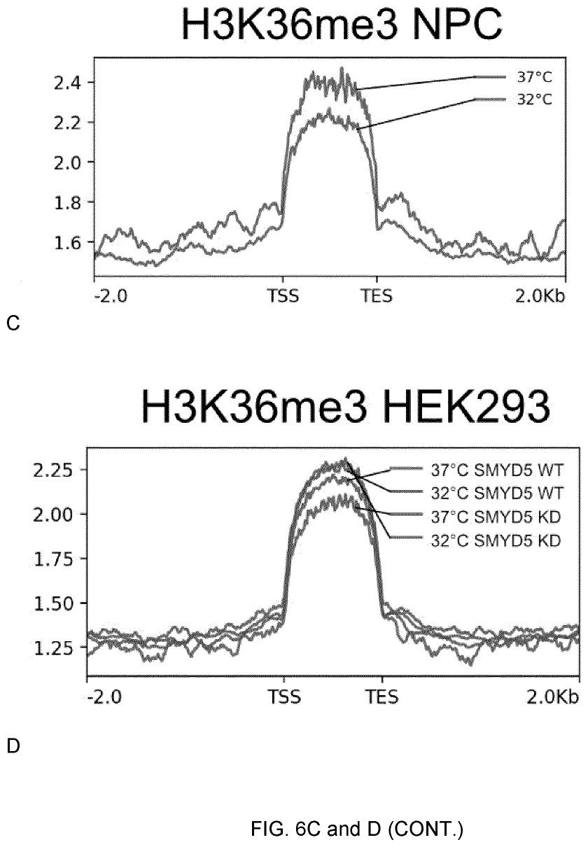

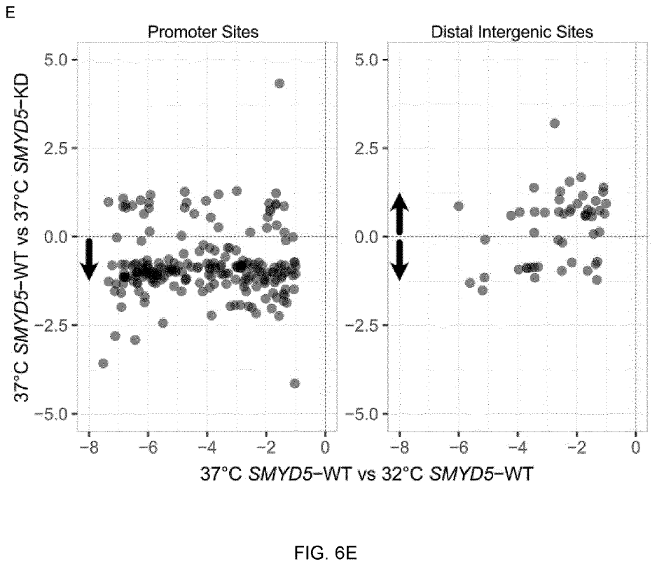

To study the epigenetic consequences of different SMYD5 availability at 37° C. versus 32° C. Cut&Run was performed using antibodies against H3K9me3, H3K36me3, H4K20me3, H3K27me3, H3K4me3 and H3 (control). Only H3K36me3 and H3K4me3 show global differences between the two temperatures (FIG. 6A-B), so we performed Cut&Run using antibodies against H3K36me3 and H3K4me3, with and without SMYD5-KD in HEK293 cells (FIG. 6A-B). We observed a lower H3K4me3 level at 32° C. compared to 37° C. for both SMYD5-knockdown (SMYD5-KD) and SMYD5-wild type (SMYD5-WT), indicating reduced protein production during cold.

Additionally, we see less H3K4me3 for SMYD5-KD compared to WT for both temperatures (FIG. 6A), indicating that SMYD5 also contributes to gene activation at both temperatures. In alignment with our prior data, we observe a decrease of H3K36me3 only at 37° C. in SMYD5-KD HEK293 cells compared to SMYD5-WT (FIG. 6B), indicating loss of H3K36me3 levels that are normally maintained at 37° C. We examined the mean Log₂ FoldChange (Log₂ FC) of H3K36me3 signal over promoter regions for each gene with significantly differential decreased H3K36me3 modifications at 32° C. When comparing this metric for two datasets, SMYD5-KD versus -WT at 37° C. and SMYD5-WT at 32° C. vs 37° C., we observe that 84% of the genes with decreased H3K36me3 at 32° C. in SMYD5-WT also have decreased H3K36me3 with SMYD5-KD at 37° C. (FIG. 6, left). In contrast, we do not observe this same phenomenon for other regions such as distal intergenic regions (FIG. 6, right). This indicates that SMYD5-KD at 37° C. induces a H3K36me3 profile that resembles the profile seen in mild hypothermia, and that this effect is primarily limited to promoters.

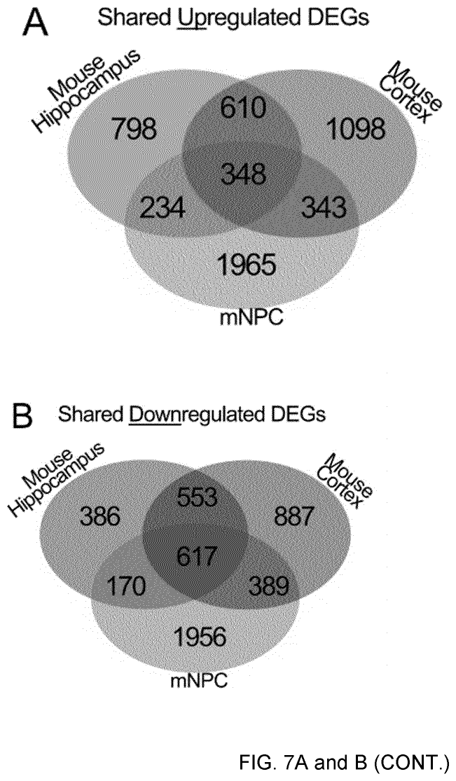

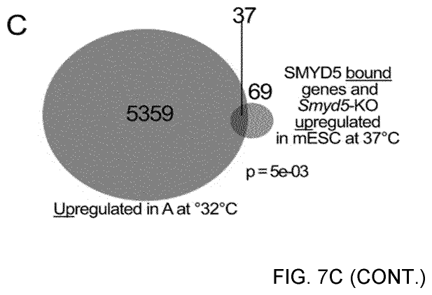

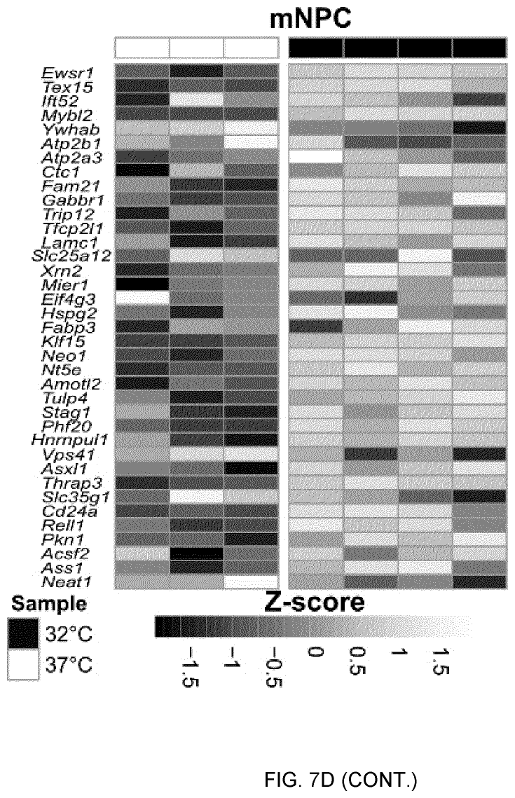

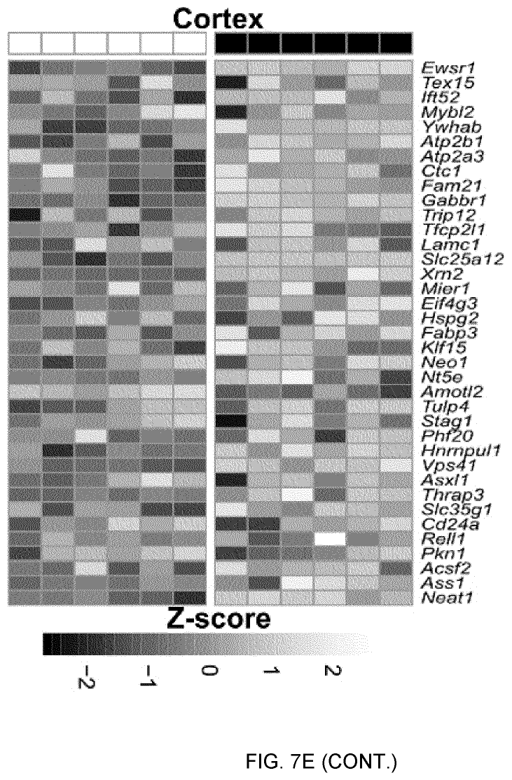

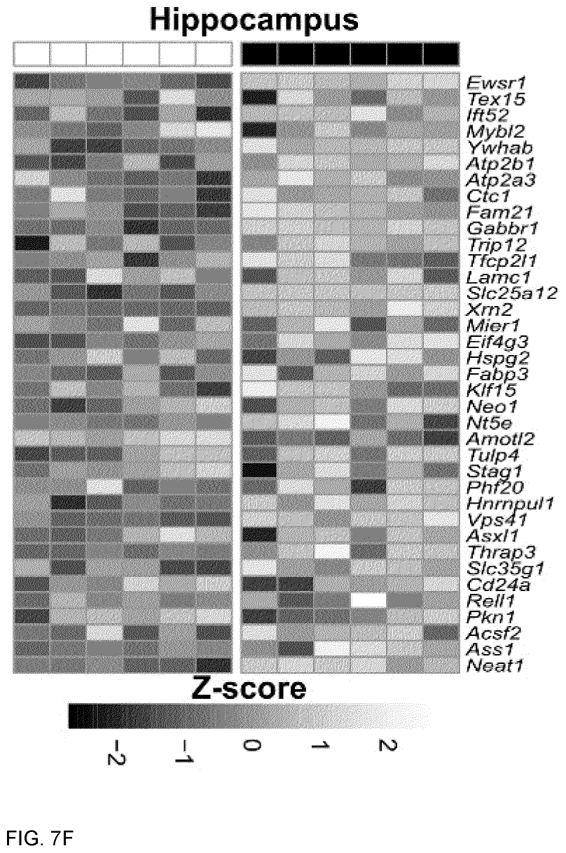

As the H3K4me3 and H3K36me3 histone modifications have been linked to alternative splicing (Luco et al., 2010; Zhu et al., 2016; Pajoro et al., 2017), the present inventors wondered if these changes may affect isoform representation and noted that SMYD5-KO cells appeared to have differing levels of the 3 known isoforms. Analysis of available RNA-Seq suggested differential isoform representation (data not shown). Importantly, this may lead to decreased stability of SMYD5 at 32° C. versus 37° C. as the shorter isoforms may show increased stability compared to constitutional isoform. By performing RNA-Seq in three distinct sources (mNPC, mouse hippocampus and mouse cortex, FIG. 7A-B) we identify 5359 upregulated genes. Among these 37 were known to be bound by and repressed by SMYD5 in mNPCs (FIG. 7C) and in general these showed less gene expression in all three tissues at 32° C. versus 37° C. (FIGS. 7D, E and F) suggesting that SMYD5 plays a general role of a regulator in the mild hypothermia response. The gene symbols of these 37 genes are: Mybl2, Hspg2, Xrn2, Fabp3, Ywhab, Phf20, Neo1, Tulp4, Trip12, Nt5e, Thrap3, Neat1, Mier1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1.

Finally, the present inventors have schematized a potential model where SMYD5 normally acts as a gatekeeper to restrict mild hypothermia response activation at 37° C., but upon degradation of SMYD5 at 32° C., SP1 and downstream players in the mild hypothermia response are enabled.

### Conclusion:

H3K36me3 was decreased at 32° C. compared to 37° C. and H3K4me3 was increased at 32° C. compared to 37° C.

### Sequence Overview

SEQ ID NO: 50: 5′ TCCCCGCC′3, MCRE sequence
SEQ ID NO: 2 to SEQ ID NO: 17 different primers used in the present disclosure
SEQ ID NO: 18 CIRBP Indicator (CIRP+GFP+Neo-pGL4.10) sequence
SEQ ID NO: 19 CIRBP-MCRE Indicator (MCRE+CIRP+GFP+Neo-pGL4.10) sequence
SEQ ID NO: 20 RBM3 Indicator (MCRE+RBM3+TurboGFP+Neo-pCMV6) sequence:
SEQ ID NO: 21 SP1 Indicator (SP1+GFP+Neo-pGL4.10) sequence
SEQ ID NO: 22 SP1-MCRE Indicator (MCRE+SP1+GFP+Neo-PGL4.10)
SEQ ID NO: 23 CIRBP lentiviral indicator (pLV[Exp]-Neo-{MCRE_spacer_CIRBP_promoter}>mCherry):
SEQ ID NO: 24 RBM3 lentiviral indicator (pLV[Exp]-Neo-{MCRE_spacer_RBM3_promoter}>mCherry):
SEQ ID NO: 25 SP1 lentiviral indicator (pLV[Exp]-Neo-{MCRE_spacer_SP1 promoter}>mCherry):
SEQ ID NO: 26 CIRBP Indicator (CIRP+GFP)
SEQ ID NO: 27 CIRP-MCRE Indicator (MCRE+CIRBP+GFP)
SEQ ID NO: 28 RBM3 Indicator (MCRE+RBM3+TurboGFP)
SEQ ID NO: 29 SP1 Indicator (SP1+GFP)
SEQ ID NO: 30 SP1-MCRE Indicator (MCRE+SP1+GF)
SEQ ID NO: 31 CIRBP indicator (MCRE_spacer_CIRBP_promoter}>mCherry)
SEQ ID NO: 32 RBM3 indicator (MCRE_spacer_RBM3_promoter}>mCherry)
SEQ ID NO: 33 SP1 indicator (MCRE_spacer_SP1_promoter}>mCherry)
SEQ ID NO: 38 CIRBP promoter
SEQ ID NO: 39 RBM3 promoter
SEQ ID NO: 40 SP1 promoter
SEQ ID NO: 41-48 linkers

### REFERENCES

Aljazi, Mohammad B et al. “SMYD5 is a histone H3-specific methyltransferase mediating mono-methylation of histone H3 lysine 36 and 37.” Biochemical and biophysical research communications vol. 599 (2022): 142-147. doi:10.1016/j.bbrc.2022.02.043
Aryee, Martin J et al. “Minfi: a flexible and comprehensive Bioconductor package for the analysis of Infinium DNA methylation microarrays.” Bioinformatics (Oxford, England) vol. 30,10 (2014): 1363-9. doi:10.1093/bioinformatics/btu049
Bastide, Amandine et al. “RTN3 Is a Novel Cold-Induced Protein and Mediates Neuroprotective Effects of RBM3.” Current biology: C B vol. 27.5 (2017): 638-650. doi:10.1016/j.cub.2017.01.047
Eller O C, Willits A B, Young E E, Baumbauer K M. Pharmacological and non-pharmacological therapeutic interventions for the treatment of spinal cord injury-induced pain. Front Pain Res (Lausanne). 2022 Aug. 24; 3:991736. doi: 10.3389/fpain.2022.991736. PMID: 36093389; PMCID: PMC9448954.
Fortin, Jean-Philippe et al. “Preprocessing, normalization and integration of the Illumina HumanMethylationEPIC array with minfi.” Bioinformatics (Oxford, England) vol. 33,4 (2017): 558-560. doi:10.1093/bioinformatics/btw691
Fujita, J. “Cold shock response in mammalian cells.” Journal of molecular microbiology and biotechnology vol. 1,2 (1999): 243-55.
Garcia et al. 2021. Use of Cryotherapy for Managing Chronic Pain: An Evidence-Based Narrative Pain Ther. 2021 June; 10(1):81-100. doi: 10.1007/s40122-020-00225-w.
Hsu Y Y, Chen C J, Wu S H, Chen K H. Cold intervention for relieving migraine symptoms: A systematic review and meta-analysis. J Clin Nurs. 2022 May 20. doi: 10.1111/jocn.16368. Epub ahead of print. PMID: 35596276.
Huttlin, Edward L et al. “Dual proteome-scale networks reveal cell-specific remodeling of the human interactome.” Cell vol. 184,11 (2021): 3022-3040.e28. doi:10.1016/j.cell.2021.04.011
Kidder, Benjamin L et al. “SMYD5 Controls Heterochromatin and Chromosome Integrity during Embryonic Stem Cell Differentiation.” Cancer research vol. 77,23 (2017): 6729-6745. doi:10.1158/0008-5472.CAN-17-0828
Kim, Dong-Hwan et al. “Vernalization: winter and the timing of flowering in plants.” Annual review of cell and developmental biology vol. 25 (2009): 277-99. doi:10.1146/annurev.cellbio.042308.113411
Kutner, Robert H et al. “Production, concentration and titration of pseudotyped HIV-1-based lentiviral vectors.” Nature protocols vol. 4,4 (2009): 495-505. doi:10.1038/nprot.2009.22
Joung, Julia et al. “Genome-scale CRISPR-Cas9 knockout and transcriptional activation screening.” Nature protocols vol. 12,4 (2017): 828-863. doi:10.1038/nprot.2017.016
Langmead, Ben, and Steven L Salzberg. “Fast gapped-read alignment with Bowtie 2.” Nature methods vol. 9,4 357-9. 4 Mar. 2012, doi:10.1038/nmeth.1923
Leek J T, et al. (2022). sva: Surrogate Variable Analysis. R package version 3.44.0.
Li, Heng et al. “The Sequence Alignment/Map format and SAMtools.” Bioinformatics (Oxford, England) vol. 25,16 (2009): 2078-9. doi:10.1093/bioinformatics/btp352
Li, Wei et al. “MAGeCK enables robust identification of essential genes from genome-scale CRISPR/Cas9 knockout screens.” Genome biology vol. 15,12 (2014): 554. doi:10.1186/s13059-014-0554-4
Luco, Reini F et al. “Epigenetics in alternative pre-mRNA splicing.” Cell vol. 144,1 (2011): 16-26. doi:10.1016/j.cell.2010.11.056
Pajoro, A et al. “Histone H3 lysine 36 methylation affects temperature-induced alternative splicing and flowering in plants.” Genome biology vol. 18,1 102. 1 Jun. 2017, doi:10.1186/s13059-017-1235-x
Luedke, M. W., Graffagnino, C., McKinney, B. G., Piper, J., Iversen, E., and Kolls, B. (2022). Association of time-temperature curves with outcomes in temperature management for cardiac arrest. BMJ Neurol Open 4. 10.1136/BMJNO-2022-000273.
Mathew, J. L., Kaur, N., and Dsouza, J. M. (2022). Therapeutic hypothermia in neonatal hypoxic encephalopathy: A systematic review and meta-analysis. J Glob Health 12. 10.7189/JOGH.12.04030.
Polderman et al. (2008), Induced hypothermia and fever control for prevention and treatment of neurological injuries. Lancet 7; 371(9628):1955-69. doi: 10.1016/S0140-6736(08)60837-5.
Quinlan, Aaron R, and Ira M Hall. “BEDTools: a flexible suite of utilities for comparing genomic features.” Bioinformatics (Oxford, England) vol. 26,6 (2010): 841-2. doi:10.1093/bioinformatics/btq033
R Core Team (2022). R: A language and environment for statistical computing. R Foundation for Statistical Computing, Vienna, Austria. URL https://www.R-project.org/.
Ritchie, Matthew E et al. “limma powers differential expression analyses for RNA-sequencing and microarray studies.” Nucleic acids research vol. 43,7 (2015): e47. doi:10.1093/nar/gkv007
Ryu, H., Lee, J., Olofsson, B. A., Mwidau, A., Deodoglu, A., Escudero, M., Flemington, E., Azizkhan-Clifford, J., Ferrante, R. J., and Ratan, R. R. (2003). Histone deacetylase inhibitors prevent oxidative neuronal death independent of expanded polyglutamine repeats via an Sp1-dependent pathway. Proc Natl Acad Sci USA 100, 4281-4286. 10.1073/PNAS.0737363100.
Sanjana, Neville E et al. “Improved vectors and genome-wide libraries for CRISPR screening.” Nature methods vol. 11,8 (2014): 783-784. doi:10.1038/nmeth.3047
Shalem, Ophir et al. “Genome-scale CRISPR-Cas9 knockout screening in human cells.” Science (New York, N.Y.) vol. 343,6166 (2014): 84-87. doi:10.1126/science.1247005
Smetanay et al. COOLHAIR: a prospective randomized trial to investigate the efficacy and tolerability of scalp cooling in patients undergoing (neo)adjuvant chemotherapy for early breast cancer. Breast Cancer Res Treat. 2019 January; 173(1):135-143.
Sumitomo, Yasuhiko et al. “Identification of a novel enhancer that binds Sp1 and contributes to induction of cold-inducible RNA-binding protein (cirp) expression in mammalian cells.” BMC biotechnology vol. 12 72. 10 Oct. 2012, doi:10.1186/1472-6750-12-72
Tan et al. 2020. Moderate Hypothermia Effectively Alleviates Acetaminophen-Induced Liver Injury With Prolonged Action Beyond Cooling. Dose-Response: An International Journal October-December 2020:1-15
Thayyil S, et al. (2021) HELIX consortium. Hypothermia for moderate or severe neonatal encephalopathy in low-income and middle-income countries (HELIX): a randomised controlled trial in India, Sri Lanka, and Bangladesh. Lancet Glob Health. 2021, 9, e1273-e1285. doi: 10.1016/S2214-109X(21)00264-3
Triche, Timothy J Jr et al. “Low-level processing of Illumina Infinium DNA Methylation BeadArrays.” Nucleic acids research vol. 41,7 (2013): e90. doi:10.1093/nar/gkt090
Xiang, Jinmei et al. “Is Hydrogen Peroxide a Suitable Apoptosis Inducer for All Cell Types?.” BioMed research international vol. 2016 (2016): 7343965. doi:10.1155/2016/7343965
Wang C Y, Ni Y, Liu Y, et al. Mild hypothermia protects liver against ischemia and reperfusion injury. World J Gastroenterol. 2005; 11(19):3005-3007. doi:10.3748/wjg.v11.i19.3005
Weber, Ceri et al. “Temperature-dependent sex determination is mediated by pSTAT3 repression of Kdm6b.” Science (New York, N.Y.) vol. 368,6488 (2020): 303-306. doi:10.1126/science.aaz4165
Wierstra, I. (2008). Sp1: Emerging roles—Beyond constitutive activation of TATA-less housekeeping genes. Biochem Biophys Res Commun 372, 1-13. 10.1016/J.BBRC.2008.03.074.
Zhang, Yanjun et al. “SMYD5 catalyzes histone H3 lysine 36 trimethylation at promoters.” Nature communications vol. 13,1 3190. 9 Jun. 2022, doi:10.1038/s41467-022-30940-1
Zhu, Xinzhou et al. “Cold-inducible proteins CIRP and RBM3, a unique couple with activities far beyond the cold.” Cellular and molecular life sciences: CMLS vol. 73,20 (2016): 3839-59. doi:10.1007/s00018-016-2253-7

### Items

1. A composition comprising a compound selected from the group consisting of catechol-O-methyltransferase (COMT) inhibitor and a tyrosine kinase inhibitor, or a pharmaceutically acceptable salt thereof for use in the treatment of a disease or condition selected from the group consisting of neonatal asphyxia, stroke, ischemia, cardiac arrest, migraine, traumatic brain injury, recovery after intense exercise, drug overdose, cerebral haemorrhage, subarachnoid haemorrhage (SAH), subdural hematoma (SH), cerebral infarction, cerebral vasospasm, spinal injury, neonatal abusive head trauma, brachial plexopathy, hair loss and neurodegeneration, in an individual in need thereof.
2. The composition for use according to the preceding item, wherein the catechol-O-methyltransferase (COMT) inhibitor is selected from the group consisting of Entacapone, Nebicapone, Nitecapone, Opicapone and Tolcapone.
3. The composition for use according to item 1, wherein the tyrosine kinase inhibitor is Poziotinib or other tyrosine kinase inhibitors such as a pan-her inhibitor, such as a pan-her inhibitor selected from the group consisting of afatinib, pyrotinib, sapatinib, erlotinib, dacomitinib, neratinib, cancertinib, varlitinib or monoclonal antibodies such as amivantamab.
4. The composition for use according to any one of items 1 and 2, wherein the compound is selected from the group consisting of: Entacapone and Poziotinib.
5. The composition for use according to any one of the preceding items, wherein the compound is Entacapone.
6. The composition for use according to anyone of the preceding items, wherein the compound is Poziotinib.
7. The composition for use according to the preceding item, wherein the composition comprises Entacapone and Poziotinib.
8. The composition for use according to the preceding items, wherein the disease or condition is selected from the group consisting of: neonatal asphyxia; stroke; ischemia; cardiac arrest; migraine; traumatic brain injury; recovery after intense exercise; cerebral haemorrhage; subarachnoid haemorrhage (SAH); subdural hematoma (SH); cerebral infarction; cerebral vasospasm; spinal injury; and neonatal abusive head trauma.
9. The composition for use anyone of the preceding items, wherein the disease or condition is selected from the group consisting of: neonatal asphyxia; stroke; ischemia; and cardiac arrest.
10. The composition for use anyone of the preceding items, wherein the disease or condition is selected from the group consisting of: neonatal asphyxia wherein neonatal asphyxia is neonatal abusive head trauma neonatal asphyxia; stroke; ischemia; and cardiac arrest, wherein the cardiac arrest is ventricular fibrillation cardiac arrest.
11. The composition for use anyone of the preceding items, wherein neonatal asphyxia is neonatal abusive head trauma neonatal asphyxia.
12. The composition for use anyone of the preceding items, the cardiac arrest is ventricular fibrillation cardiac arrest.
13. The composition for use according to any one of the preceding claims, wherein said composition for use is a substitute for Targeted Temperature Management therapy and/or hypothermia therapy.
14. The composition for use according to the preceding items, wherein the Targeted Temperature Management therapy and/or the hypothermia therapy is a therapy wherein the internal core temperature of a subject is lowered between 3° and 36° C., such as between 3° and 35.5° C., between 3° and 35° C., between 3° and 34.5° C., between 3° and 34° C., between 3° and 33.5° C., between 3° and 33° C., between 3° and 32.5° C., between 3° and 32° C., between 3° and 31.5° C., between 3° and 31° C., between 3° and 30.5° C., between 30.5 and 36° C., between 30.5 and 35.5° C., between 30.5 and 35° C., between 30.5 and 34.5° C., between 30.5 and 34° C., between 30.5 and 33.5° C., between 30.5 and 33° C., between 30.5 and 32.5° C., between 30.5 and 32° C., between 30.5 and 31.5° C., between 30.5 and 31° C., between 31 and 36° C., between 31 and 35.5° C., between 31 and 35° C., between 31 and 34.5° C., between 31 and 34° C., between 31 and 33.5° C., between 31 and 33° C., between 31 and 32.5° C., between 31 and 32° C., between 31 and 31.5° C., between 31.5 and 36° C., between 31.5 and 35.5° C., between 31.5 and 35° C., between 31.5 and 34.5° C., between 31.5 and 34° C., between 31.5 and 33.5° C., between 31.5 and 33° C., between 31.5 and 32.5° C., between 31.5 and 32° C., between 32 and 36° C., between 32 and 35.5° C., between 32 and 35° C., between 32 and 34.5° C., between 32 and 34° C., between 32 and 33.5° C., between 32 and 33° C., between 32 and 32.5° C., between 32.5 and 36° C., between 32.5 and 35.5° C., between 32.5 and 35° C., between 32.5 and 34.5° C., between 32.5 and 34° C., between 32.5 and 33.5° C., between 32.5 and 33° C., between 33 and 36° C., between 33 and 35.5° C., between 33 and 35° C., between 33 and 34.5° C., between 33 and 34° C., between 33 and 33.5° C., between 33.5 and 36° C., between 33.5 and 35.5° C., between 33.5 and 35° C., between 33.5 and 34.5° C., between 33.5 and 34° C., between 34 and 36° C., between 34 and 35.5° C., between 34 and 35° C., between 34 and 34.5° C., between 34.5 and 36° C., between 34.5 and 35.5° C., between 34.5 and 35° C., between 35 and 36° C., between 35 and 35.5° C., or between 35.5 and 36° C.
15. The composition for use according to any one of the preceding items, wherein the Targeted Temperature Management therapy and/or the hypothermia therapy is a therapy wherein the internal core temperature of a subject is lowered between 3° and 36° C., such as between 3° and 35° C., between 3° and 34° C., between 3° and 33° C., between 3° and 32° C., between 30 and 31° C., between 31 and 36° C., between 31 and 35° C., between 31 and 34° C., between 31 and 33° C., between 31 and 32° C., between 32 and 36° C., between 32 and 35° C., between 32 and 34° C., between 32 and 33° C., between 33 and 36° C., between 33 and 35° C., between 33 and 34° C., between 34 and 36° C., between 34 and 35° C., or between 35 and 36° C.
16. The composition for use according to any one of the preceding claims, wherein said composition induces a mild hypothermia response while keeping the body temperature of the individual at 36° C. or higher.
17. The composition for use according to item 16, wherein the body temperature is kept between 36 and 40° C., such as between 36 and 36.5° C., between 36 and 37° C., between 36 and 37.5° C., between 36 and 38° C., between 36 and 38.5° C., between 36 and 39° C., between 36 and 39.5° C.
18. The composition for use according to any one of the preceding items, wherein said composition induces a mild hypothermia response wherein the individual has a body temperature of 36° C. or higher at the time of administration.
19. The composition for use according to item 8, wherein the individual has a body temperature between 36 and 40° C., such as between 36 and 36.5° C., between 36 and 37° C., between 36 and 37.5° C., between 36 and 38° C., between 36 and 38.5° C., between 36 and 39° C., between 36 and 39.5° C.
20. The composition for use according to any one of the preceding items, wherein said composition induces a Mild Cold Response and wherein the individual has a body temperature of 36° C. or higher after administration of the composition, such as 1 hour or more after administration of the composition, such as 2 hours or more after administration of the composition, such as 3 hours or more after administration of the composition, such as 5 hours or more after administration of the composition, such as 8 hours or more after administration of the composition.
21. The composition for use according to any one of the preceding items, wherein the composition reduces neuronal death in the individual.
22. A composition for use in decreasing neuronal death in an individual in need thereof, wherein the composition comprises a compound selected from the group consisting of a catechol-O-methyltransferase inhibitor, such as Entacapone, Nebicapone, Nitecapone, Opicapone, Tolcapone; and a tyrosine kinase inhibitor, such as Poziotinib, and wherein the individual suffers from a disease or condition selected from the group consisting of: neonatal asphyxia, stroke, ischemia, cardiac arrest such as ventricular fibrillation cardiac arrest, migraine, traumatic brain injury, recovery after intense exercise, drug overdose, cerebral haemorrhage, subarachnoid hemorrhage (SAH), subdural hematoma (SH), cerebral infarction, cerebral vasospasm, spinal injury, neonatal abusive head trauma, brachial plexopathy, and, hair loss.
23. The composition for use according to item 22, wherein the disease or condition is selected from the group consisting of: neonatal asphyxia, stroke, ischemia, and cardiac arrest.
24. The composition for use according to any one of the preceding items, wherein the compound induces transcription of at least one gene selected from the group consisting of: SP1, CIRBP, RBM3.
25. The composition for use according to any one of the preceding items, wherein the compound increases transcription of SP1.
26. The composition for use according to any one of the preceding items, wherein the compound increases transcription of CIRBP.
27. The composition for use according to any one of the preceding items, wherein the compound increases transcription of RBM3.
28. The composition for use according to any one of the preceding items, wherein the compound is selected from the group consisting of: Entacapone, Nebicapone, Nitecapone, Opicapone, Tolcapone.
29. The composition for use according to any one of the preceding items, wherein the compound is Entacapone.
30. The composition for use according to any one of the preceding items, wherein the compound is Poziotinib.
31. The composition for use according to any one of the preceding items, wherein the composition further comprises a pharmaceutically acceptable carrier.
32. A composition for use in the treatment of a disease or condition selected from the group consisting of neonatal asphyxia, stroke, ischemia, cardiac arrest such as ventricular fibrillation cardiac arrest, migraine, traumatic brain injury, recovery after intense exercise, drug overdose, cerebral haemorrhage, subarachnoid hemorrhage (SAH), subdural hematoma (SH), cerebral infarction, cerebral vasospasm, spinal injury, neonatal abusive head trauma, brachial plexopathy, and, hair loss, in an individual in need thereof, the composition comprising an inhibitor selected from the group consisting of: SMYD5 inhibitor and a BAZ2A inhibitor.
33. The composition for use according to item 32, wherein the inhibitor is a SMYD5 inhibitor.
34. The composition for use according to any one of items 32-33, wherein the inhibitor is a BAZ2A inhibitor.
35. The composition for use according to any one of items 32-34, wherein the SMYD5 inhibitor reduces the expression of SMYD5 in a mammalian cell exposed thereto.
36. The composition for use according to any one of items 32-35, wherein the SMYD5 inhibitor induces proteasomal degradation of SMYD5 in a mammalian cell exposed thereto.
37. The composition for use according to any one of items 32-36, wherein the SMYD5 inhibitor inhibits the transcriptional activity of SMYD5 in a mammalian cell exposed thereto.
38. The composition for use according to any one of items 32-37, wherein the inhibitor inhibits the binding of SMYD5 to the promoter of a gene selected from the group consisting of: SP1 and CIRBP, preferably SP1, in a mammalian cell exposed thereto.
39. The composition for use according to any one of items 32-38, wherein the inhibitor induces tri-methylation at the 4^{th} lysine residue of the histone H3 protein (H3K4me3) and/or inhibits tri-methylation at the 36^{th} lysine residue of the histone H3 protein (H3K36me3) at the promoter of a gene selected from the group consisting of: SP1 and CIRBP, preferably SP1, in a mammalian cell exposed thereto.
40. The composition for use according to any one of items 32-39, wherein the inhibitor induces H3K4me3 at the promoter of a gene selected from the group consisting of: SP1 and CIRBP, preferably SP1, in a cell exposed thereto.
41. The composition for use according to any one of items 32-40, wherein the inhibitor inhibits H3K36me3 at the promoter of a gene selected from the group consisting of: SP1 and CIRBP, preferably SP1, in a cell exposed thereto.
42. A method of inducing a mild hypothermia response in an individual in need thereof, said method comprising inhibiting SMYD5 and/or inhibiting BAZ2A in said individual.
43. The method according to item 42, wherein the method comprises:
   a) reducing the expression of SMYD5 in a cell of the individual;
   b) promoting proteasomal degradation of SMYD5 in a cell of the individual; and/or
   c) inhibiting the transcriptional activity of SMYD5 in a cell of the individual; and/or
   d) inhibiting the binding of SMYD5 to the promoter of a gene selected from the group consisting of: SP1 and CIRBP, preferably SP1, in a cell of the individual.
44. The method according to any one of items 42-43, wherein the method comprises promoting tri-methylation at the 4th lysine residue of the histone H3 protein (H3K4me3) and/or inhibiting tri-methylation at the 36th lysine residue of the histone H3 protein (H3K36me3) at the promoter of a gene selected from the group consisting of: SP1 and CIRBP, preferably SP1, in a cell of the individual.
45. A method for determining whether an element contributes to a mild hypothermia response in an individual in need thereof, wherein the element is selected from the group consisting of: a RNA, such a siRNA, such as a snoRNA; a protein; a post-translational modification such as a histone modification; and a drug, the method comprising the steps of:
   a) quantifying the amount of the protein encoded by SMYD5 in a first sample, wherein said sample is obtained from the individual before the element is administered to the individual;
   b) administering the element to the individual;
   c) quantifying the amount of the protein encoded by SMYD5 in a second sample obtained from the same individual as in a), wherein said second sample is obtained after administration of said element; and
   d) comparing the amount of a protein encoded by SMYD5 in the samples of a) and b);
wherein a higher or lower amount of protein encoded by SMYD5 in b) than in a) is indicative of whether the element contributes to the mild hypothermia response.
46. The method according to item 45, wherein an amount of the protein encoded by SMYD5 in b) lower than the amount of the protein encoded by SMYD5 in a) is indicative of the element inducing the mild hypothermia response.
47. The method according to item 45, wherein an amount of the protein encoded by SMYD5 in b) higher than the amount of the protein encoded by SMYD5 in a) is indicative of the element repressing the mild hypothermia response.
48. The method according to any one of items 45-47, wherein the element contributes to the mild hypothermia response from treatment with the element if the amount of the protein encoded by SMYD5 in b) is at least 20%, such as at least 25%, such as at least 30%, such as at least 35%, such as at least 40%, such as at least 45%, such as at least 50% lower or higher than in a).
49. A method for monitoring activation of a mild hypothermia response in an individual following an administration of a treatment, the method comprising the steps of:
   a) quantifying the amount of the protein encoded by a gene selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1, as well as combinations thereof, in a first sample, wherein said first sample is obtained from the individual before said treatment;
   b) quantifying the amount of the protein encoded by a gene selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1, as well as combinations thereof in a second sample obtained from the same individual as in a), wherein said second sample is obtained after administration of said treatment; and
   c) comparing the amount of a protein quantified in the samples of a) and b);
   d) concluding that the mild hypothermia response is activated by the treatment if the amount of the protein encoded by a gene selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1, as well as combinations thereof in b) is higher than in a),
wherein a higher amount of protein encoded by a gene selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2/1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 as well as combinations thereof in b) than in a) is indicative of that the treatment activates the mild hypothermia response.
50. The method according to item 48, wherein the mild hypothermia response is activated in response to
   a. administration of the composition for use according to any one of claims 1 to 41; and/or
   b. administration of a Targeted Temperature Management (TTM) therapy and/or a hypothermia therapy.
51. The method according to anyone of items 49 to 50, wherein the amount of protein encoded by one or more of the listed genes is quantified by means of qPCR or qRT-PCR.
52. The method according to anyone of items 49 to 51, wherein the sample is a blood sample, or CSF sample, preferably a blood sample.
53. The composition for use according to any one of items 1 to 41, and/or the method according to any one of items 42 to 51, wherein the individual is a mammal, such as wherein the individual is a human, a mouse, a rat, a dog, a cat, a horse, a primate.
54. A mild hypothermia indicator (MHI) construct comprising:
b) a promoter sequence of a gene selected from the group consisting of SP1, RBM3 and CIRBP; and
c) a nucleic acid sequence encoding a detectable protein, wherein the detectable protein is selected from the group consisting of a fluorescent protein and a luminescent protein.
55. The MHI construct according to item 54, wherein the promoter sequence is of a mammalian gene selected from the group consisting of SP1, RBM3 and CIRBP.
56. The MHI construct according to any one of items 54 and 55, wherein the promoter sequence is of a human or murine gene selected from the group consisting of SP1, RBM3 and CIRBP.
57. The MHI construct according to any one of items 54 to 56, wherein the MHI construct further comprises a linker sequence.
58. The MHI construct according to any one of items 54 to 57, wherein the linker sequence is positioned between the promoter sequence and the nucleic acid encoding a detectable protein.
59. The MHI construct according to any one of items 54 to 58, wherein the linker sequence is attached to the 3′ of the promoter sequence and to the 5′ of the nucleic acid sequence encoding a detectable protein.
60. The MHI construct according to any one of items 54 to 59, wherein the linker sequence is a nucleic acid sequence comprising between and 200 nucleotides.
61. The MHI construct according to any one of items 54 to 60, wherein the detectable protein is a fluorescent protein.
62. The MHI construct according to any one of items 54 to 61, wherein the detectable protein is a fluorescent protein selected from the group consisting of Green Fluorescent Protein (GFP) and derivatives thereof, and monomeric red fluorescent protein (RFP) and derivatives thereof.
63. The MHI construct according to any one of items 54 to 62, wherein the fluorescent protein is selected from the group consisting of Green Fluorescent Protein (GFP), yellow fluorescent protein (YFP), cyan fluorescent protein (CFP), monomeric blue fluorescent protein (BFP), Aequorea coerulescens GFP1 (AcGFP or AcGFP1), enhanced GFP (EGFP), TurboGFP, RFP, mRFP1, mCherry, mStrawberry, mOrange, dTomato.
64. The MHI construct according to any one of items 54 to 63, wherein the detectable protein is a luminescent protein.
65. The MHI construct according to item 64, wherein the luminescent protein is luciferase.
66. The MHI construct according to any one of items 54 to 65, further comprising a response enhancer.
67. The MHI construct according to item 66, wherein the response enhancer comprises or consists of one or more copies of a Mild Cold Responsive Element (MCRE) sequence,
68. The MHI construct according to any one of items 66 to 67, wherein the MCRE sequence consists of SEQ ID NO: 50: 5′ TCCCCGCC′3, or a variant thereof, wherein said variant differs from SEQ ID NO: 50: 5′ TCCCCGCC′3, of one nucleotide.
69. The MHI construct according to any one of items 66 to 68, wherein the response enhancer comprises or consists of between 2 and 20 copies of the MCRE sequence of SEQ ID NO: 50: 5′ TCCCCGCC′3, or a variant thereof.
70. The MHI construct according to any one of items 66 to 69, wherein the response enhancer is positioned at the 5′ of the promoter sequence, such as at the 3′ of the promoter sequence.
71. The MHI construct according to any one of items 66 to 70, further comprising a spacer sequence positioned between the response enhancer and the promoter sequence.
72. The MHI construct according to item 71, wherein the spacer sequence comprises or consists of 1 to 200 nucleotides.
73. The MHI construct according to any one of items 54 to 72, further comprising a selection cassette.
74. The MHI construct according to item 73, wherein the selection cassette is positioned at the 3′ of the nucleic acid sequence encoding the detectable protein.
75. The MHI construct according to any one of items 73 and 74, wherein the selection cassette is a neomycin selection cassette.
76. The MHI construct according to any one of items 54 to 75, wherein said construct consists of a promoter sequence, a linker sequence and a nucleic acid encoding a detectable protein.
77. The MHI construct according to any one of items 54 to 76, wherein said construct consists of a response enhancer, a spacer sequence, a promoter sequence, a linker sequence and a nucleic acid encoding a detectable protein.
78. The MHI construct according to any one of items 54 to 77, wherein said construct comprises a nucleic acid sequence selected from the group consisting of: SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or a functional variant thereof, wherein said functional variant has at least 80% sequence identity to any one of SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33.
79. The MHI construct according to any one of items 54 to 78, wherein said construct consists of a nucleic acid sequence selected from the group consisting of: SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or a functional variant thereof, wherein said functional variant has at least 80% sequence identity to any one of SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33.
80. An expression vector comprising an expression cassette encoding a MHI construct according to any one of items 54 to 79.
81. The expression vector according to item 80, wherein said expression vector is a plasmid.
82. The expression vector according to item 80, wherein said expression vector is a viral vector selected from the group consisting of an adeno-associated viral vector, an adenoviral vector, a lentiviral vector or a retroviral vector.
83. The expression vector according to any one of items 80 to 82, further comprising a selection cassette.
84. The expression vector according to any one of items 80 to 83, said expression vector comprising a nucleic acid sequence selected from the group consisting of: SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25, or a functional variant thereof, wherein said functional variant has at least 80% sequence identity to any one of SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25.
85. The expression vector according to any one of items 80 to 84, said expression vector consisting of a nucleic acid sequence selected from the group consisting of: SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25, or a functional variant thereof, wherein said functional variant has at least 80% sequence identity to any one of SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25.
86. A host cell transformed, transfected or transduced with the expression vector according to any one of items 80 to 85.
87. An in vitro method for monitoring activation the mild hypothermia response in a biological sample in response to a treatment, the method comprising the steps of:
   a. providing a first biological sample obtained from a mammal before administration of the treatment,
   b. contacting the first biological sample with an MHI construct according to any one of items 54 to 79,
   c. determining the level of fluorescence or luminescence in the first biological sample,
   d. providing a second biological sample obtained from the same mammal after administration of the treatment,
   e. contacting the second biological sample with an MHI construct according to any one of items 54 to 79,
   f. determining the level of fluorescence or luminescence in the second biological sample,
wherein the mild hypothermia response is activated by the treatment if the fluorescence in the second biological sample is higher than in the first biological sample.
88. The method according to item 87, wherein the biological sample is selected from the group consisting of cells and tissues.
89. The method according to any one of items 87 and 88, further comprising:
   g. providing a third or further biological sample obtained from the same mammal after administration of the treatment,
   h. contacting the third or further biological sample with an MHI construct according to any one of items 54 to 79,
   i. determining the level of fluorescence or luminescence in the third or further biological sample,
   thereby monitoring the activation, progression or regression of a mild hypothermia response.
90. The method according to any one of items 87 to 869, wherein the biological sample is a blood sample or a cerebral spinal fluid (CSF) sample.
91. The method according to any one of items 87 to 90, wherein fluorescence or luminescence is measured at least 15 minutes, such as of at least 20 minutes, such as of at least 30 minutes, such as of at least 45 minutes, such as of at least 60 minutes, such as of at least 2 hours, such as of at least 4 hours, such as of at least 8 hours, such as of at least 12 hours, such as of at least 16 hours after administration of the treatment.
92. The method according to any one of items 87 to 91, wherein fluorescence or luminescence is measured multiple times after administration of the treatment.
93. An in vitro method for evaluating the effect of a candidate pharmacological agent for treatment of a disease or condition selected from the group consisting of: neonatal asphyxia, stroke, ischemia, cardiac arrest, migraine, traumatic brain injury, recovery after intense exercise, drug overdose, cerebral haemorrhage, subarachnoid haemorrhage (SAH), subdural hematoma (SH), cerebral infarction, cerebral vasospasm, spinal injury, neonatal abusive head trauma, brachial plexopathy, hair loss due to chemotherapy, chronic pain, neuropathic pain, and neurological pain, in an individual in need thereof, the method comprising the steps of:
a) providing mammalian cells transfected with a MHI construct according to any one of items 54 to 79,
b) measuring fluorescence or luminescence of the cells,
c) contacting the cells with a candidate pharmacological agent,
d) measuring fluorescence or luminescence of the cells after exposure to the candidate pharmacological agent,
   wherein a higher fluorescence or luminescence of the cells after exposure to the candidate pharmacological agent is indicative of the agent having an effect for treatment of said disease or condition.
94. The method according to any one of items 87 to 93, wherein fluorescence or luminescence is measured after an exposure to a pharmacological agent of at least 15 minutes, such as of at least 20 minutes, such as of at least 30 minutes, such as of at least 45 minutes, such as of at least 60 minutes, such as of at least 2 hours, such as of at least 4 hours, such as of at least 8 hours, such as of at least 12 hours, such as of at least 16 hours.
95. The method according to any one of items 87 to 94, wherein fluorescence or luminescence is measured multiple times during and/or after an exposure to a pharmacological agent, for example 1 hour, 2 hours, 4 hours, 8 hours, 12 hours, 16 hours, 20 hours, 24 hours, 28 hours, 32 hours, 36 hours, 40 hours, 48 hours after the start of the exposure, and any time points in between.
96. The method according to any one of items 87 to 95, wherein a fluorescence or luminescence of the cells of at least 2-fold higher, such as at least 2.25-fold higher, such as at least 2.5-fold higher, such as at least 2.75-fold higher, such as at least 3-fold higher, such as at least 3.25-fold higher, such as at least 3.5-fold higher, such as at least 3.75-fold higher after exposure to the candidate pharmacological agent compared to in a), is indicative of the agent having an effect for treatment of said disease or condition.
97. The method according to any one of items 87 to 96, wherein fluorescence is measured with flow cytometry and/or with fluorescence microscopy, and wherein luminescence is measured with a luminometer.

1. A composition comprising a compound selected from the group consisting of: Entacapone and Poziotinib, or a pharmaceutically acceptable salt thereof for use in the treatment of a disease or condition selected from the group consisting of: neonatal asphyxia, cerebral haemorrhage, subarachnoid haemorrhage (SAH), subdural hematoma (SH), cerebral infarction, cerebral vasospasm, in an individual in need thereof.

2. A composition comprising a compound selected from the group consisting of: Entacapone and Poziotinib, or a pharmaceutically acceptable salt thereof for use in the treatment of a disease or condition selected from the group consisting of: stroke, ischemia, cardiac arrest, migraine, traumatic brain injury, recovery after intense exercise, drug overdose, spinal injury, and non-accidental paediatric abusive head trauma, brachial plexopathy, hair loss and neurodegeneration, in an individual in need thereof.

3. A composition for use in decreasing neuronal death in an individual in need thereof, wherein the composition comprises a compound selected from the group consisting of: Entacapone and Poziotinib, or a pharmaceutically acceptable salt thereof, and wherein the individual suffers from a disease or condition selected from the group consisting of: neonatal asphyxia, stroke, ischemia, cardiac arrest such as ventricular fibrillation cardiac arrest, migraine, traumatic brain injury, recovery after intense exercise, drug overdose, cerebral haemorrhage, subarachnoid hemorrhage (SAH), subdural hematoma (SH), cerebral infarction, cerebral vasospasm, spinal injury, neonatal abusive head trauma, brachial plexopathy, and, hair loss.

4. The composition for use according to anyone of the preceding claims, wherein the disease or condition is selected from the group consisting of neonatal asphyxia, stroke, ischemia, and cardiac arrest.

5. The composition for use anyone of the preceding claims, wherein neonatal asphyxia is neonatal abusive head trauma neonatal asphyxia.

6. The composition for use anyone of the preceding claims, the cardiac arrest is ventricular fibrillation cardiac arrest.

7. The composition for use according to any one of claim 1 or 2, wherein said composition comprises Entacapone or a pharmaceutically acceptable salt thereof.

8. The composition for use according to any one of the preceding claims, wherein said composition for use is a substitute for Targeted Temperature Management therapy and/or hypothermia therapy.

9. The composition for use according to the preceding claims, wherein the Targeted Temperature Management therapy and/or the hypothermia therapy is a therapy wherein the internal core temperature of a subject is lowered between 3° and 36° C., such as between 3° and 35.5° C., between 3° and 35° C., between 3° and 34.5° C., between 3° and 34° C., between 3° and 33.5° C., between 3° and 33° C., between 3° and 32.5° C., between 3° and 32° C., between 3° and 31.5° C., between 3° and 31° C., between 3° and 30.5° C., between 30.5 and 36° C., between 30.5 and 35.5° C., between 30.5 and 35° C., between 30.5 and 34.5° C., between 30.5 and 34° C., between 30.5 and 33.5° C., between 30.5 and 33° C., between 30.5 and 32.5° C., between 30.5 and 32° C., between 30.5 and 31.5° C., between 30.5 and 31° C., between 31 and 36° C., between 31 and 35.5° C., between 31 and 35° C., between 31 and 34.5° C., between 31 and 34° C., between 31 and 33.5° C., between 31 and 33° C., between 31 and 32.5° C., between 31 and 32° C., between 31 and 31.5° C., between 31.5 and 36° C., between 31.5 and 35.5° C., between 31.5 and 35° C., between 31.5 and 34.5° C., between 31.5 and 34° C., between 31.5 and 33.5° C., between 31.5 and 33° C., between 31.5 and 32.5° C., between 31.5 and 32° C., between 32 and 36° C., between 32 and 35.5° C., between 32 and 35° C., between 32 and 34.5° C., between 32 and 34° C., between 32 and 33.5° C., between 32 and 33° C., between 32 and 32.5° C., between 32.5 and 36° C., between 32.5 and 35.5° C., between 32.5 and 35° C., between 32.5 and 34.5° C., between 32.5 and 34° C., between 32.5 and 33.5° C., between 32.5 and 33° C., between 33 and 36° C., between 33 and 35.5° C., between 33 and 35° C., between 33 and 34.5° C., between 33 and 34° C., between 33 and 33.5° C., between 33.5 and 36° C., between 33.5 and 35.5° C., between 33.5 and 35° C., between 33.5 and 34.5° C., between 33.5 and 34° C., between 34 and 36° C., between 34 and 35.5° C., between 34 and 35° C., between 34 and 34.5° C., between 34.5 and 36° C., between 34.5 and 35.5° C., between 34.5 and 35° C., between 35 and 36° C., between 35 and 35.5° C., or between 35.5 and 36° C.

10. The composition for use according to any one of the preceding claims, wherein the Targeted Temperature Management therapy and/or the hypothermia therapy is a therapy wherein the internal core temperature of a subject is lowered between 3° and 36° C., such as between 3° and 35° C., between 3° and 34° C., between 3° and 33° C., between 3° and 32° C., between 30 and 31° C., between 31 and 36° C., between 31 and 35° C., between 31 and 34° C., between 31 and 33° C., between 31 and 32° C., between 32 and 36° C., between 32 and 35° C., between 32 and 34° C., between 32 and 33° C., between 33 and 36° C., between 33 and 35° C., between 33 and 34° C., between 34 and 36° C., between 34 and 35° C., or between 35 and 36° C.

11. The composition for use according to any one of the preceding claims, wherein said composition induces a mild hypothermia response while keeping the body temperature of the individual at 36° C. or higher.

12. The composition for use according to claim 11, wherein the body temperature is kept between 36 and 40° C., such as between 36 and 36.5° C., between 36 and 37° C., between 36 and 37.5° C., between 36 and 38° C., between 36 and 38.5° C., between 36 and 39° C., between 36 and 39.5° C.

13. The composition for use according to any one of the preceding claims, wherein said composition induces a mild hypothermia response wherein the individual has a body temperature of 36° C. or higher at the time of administration.

14. The composition for use according to claim 13, wherein the individual has a body temperature between 36 and 40° C., such as between 36 and 36.5° C., between 36 and 37° C., between 36 and 37.5° C., between 36 and 38° C., between 36 and 38.5° C., between 36 and 39° C., between 36 and 39.5° C.

15. The composition for use according to any one of the preceding claims, wherein said composition induces a mild hypothermia response and wherein the individual has a body temperature of 36° C. or higher after administration of the composition, such as 1 hour or more after administration of the composition, such as 2 hours or more after administration of the composition, such as 3 hours or more after administration of the composition, such as 5 hours or more after administration of the composition, such as 8 hours or more after administration of the composition.

16. The composition for use according to any one of the preceding claims, wherein the composition increases transcription of at least one gene selected from the group consisting of: SP1, CIRBP, RBM3.

17. The composition for use according to any one of the preceding claims, wherein the compound increases transcription of SP1.

18. The composition for use according to any one of the preceding claims, wherein the compound increases transcription of CIRBP.

19. The composition for use according to any one of the preceding claims, wherein the compound increases transcription of RBM3.

20. The composition for use according to any one of the preceding claims, wherein the composition reduces neuronal death in the individual.

21. A method of inducing a mild hypothermia response in an individual in need thereof, said method comprising inhibiting SMYD5 and/or inhibiting BAZ2A in said individual.

22. The method according to claim 21, wherein the method comprises:
e) reducing the expression of SMYD5 in a cell of the individual;
f) promoting proteasomal degradation of SMYD5 in a cell of the individual; and/or
g) inhibiting the transcriptional activity of SMYD5 in a cell of the individual; and/or
h) inhibiting the binding of SMYD5 to the promoter of a gene selected from the group consisting of: SP1 and CIRBP, preferably SP1, in a cell of the individual.

23. The method according to any one of claims 21-22, wherein the method comprises promoting tri-methylation at the 4th lysine residue of the histone H3 protein (H3K4me3) and/or inhibiting tri-methylation at the 36th lysine residue of the histone H3 protein (H3K36me3) at the promoter of a gene selected from the group consisting of: SP1 and CIRBP, preferably SP1, in a cell of the individual.

24. A mild hypothermia indicator (MHI) construct comprising:
a) a promoter sequence of a gene selected from the group consisting of SP1, RBM3 and CIRBP; and
b) a nucleic acid sequence encoding a fluorescent protein.

25. The MHI construct according to claim 24, wherein the promoter sequence is of a mammalian gene selected from the group consisting of SP1, RBM3 and CIRBP.

26. The MHI construct according to any one of claims 24 and 25, wherein the promoter sequence is of a human or murine gene selected from the group consisting of SP1, RBM3 and CIRBP.

27. The MHI construct according to any one of claims 24 to 26, wherein the MHI construct further comprises:
a) a linker sequence,
b) a response enhancer and/or
c) a selection cassette.

28. The MHI construct according to any one of claims 24 to 27, wherein SP1 promoter comprises or consists of SEQ ID NO: 40 or a functional variant thereof having at least 80% sequence identity to SEQ ID NO: 40, RBM1 promoter comprises or consists of SEQ ID NO: 39 or a functional variant thereof having at least 80% sequence identity to SEQ ID NO: 39, CIRBP promoter comprises or consists of SEQ ID NO: 38 or a functional variant thereof having at least 80% sequence identity to SEQ ID NO: 39.

29. The MHI construct according to any one of claims 24 to 28, wherein the linker sequence is positioned between the promoter sequence and the nucleic acid encoding a fluorescent protein.

30. The MHI construct according to any one of claims 24 to 29, wherein the linker sequence is attached to the 3′ of the promoter sequence and to the 5′ of the nucleic acid sequence encoding a fluorescent protein.

31. The MHI construct according to any one of claims 24 to 30, wherein the linker sequence is a nucleic acid sequence comprising between and 200 nucleotides.

32. The MHI construct according to any one of claims 24 to 31, wherein the fluorescent protein is selected from the group consisting of Green Fluorescent Protein (GFP) and derivatives thereof, and monomeric red fluorescent protein (RFP) and derivatives thereof.

33. The MHI construct according to any one of claims 24 to 32, wherein the fluorescent protein is selected from the group consisting of Green Fluorescent Protein (GFP), yellow fluorescent protein (YFP), cyan fluorescent protein (CFP), monomeric blue fluorescent protein (BFP), Aequorea coerulescens GFP1 (AcGFP or AcGFP1), enhanced GFP (EGFP), TurboGFP, RFP, mRFP1, mCherry, mStrawberry, mOrange, dTomato.

34. The MHI construct according to any one of claims 24 to 33, wherein the response enhancer comprises or consists of one or more copies of a Mild Cold Responsive Element (MCRE) sequence.

35. The MHI construct according to any one of claims 24 to 34, wherein the MCRE sequence consists of SEQ ID NO: 50: 5′ TCCCCGCC′3, or a variant thereof, wherein said variant differs from SEQ ID NO: 50: 5′ TCCCCGCC′3, of one nucleotide.

36. The MHI construct according to any one of claims 24 to 35, wherein the response enhancer comprises or consists of between 2 and 20 copies of the MCRE sequence of SEQ ID NO: 50: 5′ TCCCCGCC′3, or a variant thereof.

37. The MHI construct according to any one of claims 24 to 36, wherein the response enhancer is positioned at the 5′ of the promoter sequence, such as at the 3′ of the promoter sequence.

38. The MHI construct according to any one of claims 24 to 37, further comprising a spacer sequence positioned between the response enhancer and the promoter sequence.

39. The MHI construct according to claim 38, wherein the spacer sequence comprises or consists of 1 to 200 nucleotides.

40. The MHI construct according to any one of claims 24 to 39, wherein said construct consists of a promoter sequence, a linker sequence and a nucleic acid encoding a fluorescent protein.

41. The MHI construct according to any one of claims 24 to 40, wherein said construct consists of a response enhancer, a spacer sequence, a promoter sequence, a linker sequence and a nucleic acid encoding a fluorescent protein.

42. The MHI construct according to any one of claims 24 to 41, wherein said construct comprises a nucleic acid sequence selected from the group consisting of: SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or a functional variant thereof, wherein said functional variant has at least 80% sequence identity to any one of SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33.

43. The MHI construct according to any one of claims 24 to 42, wherein said construct consists of a nucleic acid sequence selected from the group consisting of: SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or a functional variant thereof, wherein said functional variant has at least 80% sequence identity to any one of SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33.

44. An expression vector comprising an expression cassette encoding a MHI construct according to any one of claims 24 to 43.

45. The expression vector according to claim 44, wherein said expression vector is a plasmid.

46. The expression vector according to claim 44, wherein said expression vector is a viral vector selected from the group consisting of an adeno-associated viral vector, an adenoviral vector, a lentiviral vector or a retroviral vector.

47. The expression vector according to any one of claims 44 to 46, further comprising a selection cassette.

48. The expression vector according to any one of claims 44 to 47, said expression vector comprising a nucleic acid sequence selected from the group consisting of: SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25, or a functional variant thereof, wherein said functional variant has at least 80% sequence identity to any one of SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25.

49. The expression vector according to any one of claims 44 to 48, said expression vector consisting of a nucleic acid sequence selected from the group consisting of: SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25, or a functional variant thereof, wherein said functional variant has at least 80% sequence identity to any one of SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25.

50. A host cell transformed, transfected or transduced with the expression vector according to any one of claims 44 to 49.

51. An in vitro method for monitoring activation of the mild hypothermia response in a biological sample in response to a treatment, the method comprising the steps of:
a) providing a first biological sample obtained from a mammal before administration of the treatment,
b) contacting the first biological sample with an MHI construct comprising
i. a promoter sequence of a gene selected from the group consisting of SP1, RBM3 and CIRBP; and
i. a nucleic acid sequence encoding a detectable protein, wherein the detectable protein is selected from the group consisting of a fluorescent protein and a luminescent protein;
c) determining the level of fluorescence or luminescence in the first biological sample,
d) providing a second biological sample obtained from the same mammal after administration of the treatment,
e) contacting the second biological sample with an MHI construct according to b,
f) determining the level of fluorescence or luminescence in the second biological sample,
wherein the mild hypothermia response is activated by the treatment if the fluorescence in the second biological sample is higher than in the first biological sample.

52. The method according to claim 51, wherein the biological sample is selected from the group consisting of cells and tissues.

53. The method according to any one of claims 51 and 52, further comprising:
j. providing a third or further biological sample obtained from the same mammal after administration of the treatment,
k. contacting the third or further biological sample with an MHI construct according to b. or e.,
l. determining the level of fluorescence or luminescence in the third or further biological sample,
thereby monitoring the activation, progression or regression of a mild hypothermia response.

54. The method according to any one of claims 51 to 53, wherein the MHI construct is according to any one of claims 23 to 43.

55. The method according to any one of claims 51 to 54, wherein the biological sample is a blood sample or a cerebral spinal fluid (CSF) sample.

56. The method according to any one of claims 51 to 55, wherein fluorescence or luminescence is measured at least 15 minutes, such as of at least 20 minutes, such as of at least 30 minutes, such as of at least 45 minutes, such as of at least 60 minutes, such as of at least 2 hours, such as of at least 4 hours, such as of at least 8 hours, such as of at least 12 hours, such as of at least 16 hours after administration of the treatment.

57. The method according to any one of claims 51 to 56, wherein fluorescence or luminescence is measured multiple times after administration of the treatment.

58. A method for monitoring activation of a mild hypothermia response in an individual following administration of a treatment, the method comprising the steps of:
a) quantifying the amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in a first sample, wherein said first sample is obtained from the individual before said treatment;
b) quantifying the amount of the protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp2l1, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in a second sample obtained from the same individual as in a), wherein said second sample is obtained after administration of said treatment; and
c) comparing the amount of a protein quantified in a) and b);
d) concluding that the mild hypothermia response is activated by the treatment if the amount of the protein quantified in b) is lower than in a),
wherein a lower amount of protein encoded by one or more genes selected from the group consisting of: SMYD5, Mybl2, Hspg2, Xrn2, Fabp3, Ywhab, Phf20, Neo1, Tulp4, Trip12, Nt5e, Thrap3, Neat1, Rell1, Eif4g3, Ift52, Tfcp211, Fam21, Ctc1, Lamc1, Amotl2, Gabbr1, Hnrnpul1, Asxl1, Atp2b1, Atp2a3, Klf15, Cd24a, Tex15, Slc25a12, Slc35g1, Stag1, Pkn1, Vps41, Ewsr1, Acsf2, Ass1 in b) than in a) is indicative of that the treatment activates the mild hypothermia response.

59. The method according to claim 58, wherein the mild hypothermia response is activated in response to
a. administration of the composition for use according to any one of claims 1 to 23; and/or
b. administration of a Targeted Temperature Management (TTM) therapy and/or a hypothermia therapy.

60. The method according to anyone of claims 58 to 59, wherein the amount of protein encoded by one or more of the listed genes is quantified by means of qPCR or qRT-PCR.

61. The method according to anyone of claims 58 to 60, wherein the sample is a blood sample, or CSF sample, preferably a blood sample.

62. The composition for use according to any one of claims 1 to 23, and/or the method according to any one of claims 51 to 61, wherein the individual is a mammal, such as wherein the individual is a human, a mouse, a rat, a dog, a cat, a horse, a primate.

63. An in vitro method for evaluating the effect of a candidate pharmacological agent for treatment of a disease or condition selected from the group consisting of: neonatal asphyxia, stroke, ischemia, cardiac arrest, migraine, traumatic brain injury, recovery after intense exercise, drug overdose, cerebral haemorrhage, subarachnoid haemorrhage (SAH), subdural hematoma (SH), cerebral infarction, cerebral vasospasm, spinal injury, neonatal abusive head trauma, brachial plexopathy, hair loss due to chemotherapy, chronic pain, neuropathic pain, and neurological pain, in an individual in need thereof, the method comprising the steps of:
a) providing mammalian cells transfected with a MHI construct comprising
i. a promoter sequence of a gene selected from the group consisting of SP1, RBM3 and CIRBP; and
ii. a nucleic acid sequence encoding a detectable protein, wherein the detectable protein is selected from the group consisting of a fluorescent protein and a luminescent protein;
b) measuring fluorescence or luminescence of the cells,
c) contacting the cells with a candidate pharmacological agent,
d) measuring fluorescence or luminescence of the cells after exposure to the candidate pharmacological agent,
wherein a higher fluorescence or luminescence of the cells after exposure to the candidate pharmacological agent is indicative of the agent having an effect for treatment of said disease or condition.

64. The method according to claim 63, wherein the MHI construct is according to any one of claims 23 to 43.

65. The method according to any one of claims 63 and 64, wherein fluorescence or luminescence is measured after an exposure to a pharmacological agent of at least 15 minutes, such as of at least 20 minutes, such as of at least 30 minutes, such as of at least 45 minutes, such as of at least 60 minutes, such as of at least 2 hours, such as of at least 4 hours, such as of at least 8 hours, such as of at least 12 hours, such as of at least 16 hours.

66. The method according to any one of claims 63 to 65, wherein fluorescence or luminescence is measured multiple times during and/or after an exposure to a pharmacological agent, for example 1 hour, 2 hours, 4 hours, 8 hours, 12 hours, 16 hours, 20 hours, 24 hours, 28 hours, 32 hours, 36 hours, 40 hours, 48 hours after the start of the exposure, and any time points in between.

67. The method according to any one of claims 63 to 66, wherein a fluorescence or luminescence of the cells of at least 2-fold higher, such as at least 2.25-fold higher, such as at least 2.5-fold higher, such as at least 2.75-fold higher, such as at least 3-fold higher, such as at least 3.25-fold higher, such as at least 3.5-fold higher, such as at least 3.75-fold higher after exposure to the candidate pharmacological agent compared to in a), is indicative of the agent having an effect for treatment of said disease or condition.

68. The method according to any one of claims 63 to 67, wherein fluorescence is measured with flow cytometry and/or with fluorescence microscopy, and wherein luminescence is measured with a luminometer.


SEQUENCE LISTING (48 sequences)

SEQ ID NO: 1 - skipped

SEQ ID NO: 2 - DNA, 35 bp, synthetic construct
  source 1..35: mol_type = other DNA
tcgataggtacctggcttcacaaatgcgcctcagt

SEQ ID NO: 3 - DNA, 34 bp, synthetic construct
  source 1..34: mol_type = other DNA
cctaaggcagatctgcgagggggagcgcaagagt

SEQ ID NO: 4 - DNA, 39 bp, synthetic construct
  source 1..39: mol_type = other DNA
tcaagtcaggctagcgcaacttagtctcacacgccttgg

SEQ ID NO: 5 - DNA, 36 bp, synthetic construct
  source 1..36: mol_type = other DNA
cagtgctgcctcgaggctcaagggggtcctgtccgg

SEQ ID NO: 6 - DNA, 34 bp, synthetic construct
  source 1..34: mol_type = other DNA
cggcggagtctagaattacttgtacagctcgtcc

SEQ ID NO: 7 - DNA, 31 bp, synthetic construct
  source 1..31: mol_type = other DNA
taagccaccatggtgagcaagggcgaggagc

SEQ ID NO: 8 - DNA, 36 bp, synthetic construct
  source 1..36: mol_type = other DNA
cattatcgtcgactctaccgggtaggggaggcgctt

SEQ ID NO: 9 - DNA, 44 bp, synthetic construct
  source 1..44: mol_type = other DNA
cgccgccgacgatagtcaagcttctgatggaattagaacttggc

SEQ ID NO: 10 - DNA, 22 bp, synthetic construct
  source 1..22: mol_type = other DNA
cacccaattcaaggcctgccgt

SEQ ID NO: 11 - DNA, 22 bp, synthetic construct
  source 1..22: mol_type = other DNA
gggttgggcatctgggctgttt

SEQ ID NO: 12 - DNA, 22 bp, synthetic construct
  source 1..22: mol_type = other DNA
gagactcagcggtccaggggtt

SEQ ID NO: 13 - DNA, 22 bp, synthetic construct
  source 1..22: mol_type = other DNA
cctctggttccccgagcagact

SEQ ID NO: 14 - DNA, 22 bp, synthetic construct
  source 1..22: mol_type = other DNA
ccgagttgaccaggctggcaag

SEQ ID NO: 15 - DNA, 22 bp, synthetic construct
  source 1..22: mol_type = other DNA
tccatagccccggtctcctcct

SEQ ID NO: 16 - DNA, 20 bp, synthetic construct
  source 1..20: mol_type = other DNA
tcaaggctgagaacgggaag

SEQ ID NO: 17 - DNA, 20 bp, synthetic construct
  source 1..20: mol_type = other DNA
cgccccacttgattttggag

SEQ ID NO: 18 - DNA, 5571 bp, synthetic construct
  source 1..5571: mol_type = other DNA
ggcctaactggccggtacccatctgtcttctggcttcacaaatgcgcctcagttaaataactgtaattaaatattaaaagaaaaaaaagaacataaaagtgttgagtcgatgagtcaaggttggagccatatttgggagtctcattgaggtagcaaaagctctttgttccccgccgcaggacaaacgggtcgggaacgcggaagtgggtcagctccgcctccaaatcagctgtccccgcctcctagtcgctagcgggcggggggtgggggggaaaaatgcggccaggaaagattccagccaatcaaaacactcagcgcctccgcgcggactagggcaaagccaacggagtcccgcccctccaccgaggcgcagatgccgaatctaaggcgtcggattggtcagcgcggcgtggtgggcgggttaaggggcgtggctccaggagtggagtatatcaggcgggactcttgcgctccccctcgcactcgcgccttaggaagcttgggtgtgtgtggcgcgctgtcttcccgctcgcgtcagggacctgcccgactcagcggtgagggagatctggcctcggcggccaagcttggcaatccggtactgttggtaaagccaccatggatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtgatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgactctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 19 - DNA, 5657 bp, synthetic construct
  source 1..5657: mol_type = other DNA
ggcctaactggccggtaccttccccgccgttccccgccgttccccgccgttccccgccgttccccgccgtctggctaactagagaacccactgcgccacagtacccatctgtcttctggcttcacaaatgcgcctcagttaaataactgtaattaaatattaaaagaaaaaaaagaacataaaagtgttgagtcgatgagtcaaggttggagccatatttgggagtctcattgaggtagcaaaagctctttgttccccgccgcaggacaaacgggtcgggaacgcggaagtgggtcagctccgcctccaaatcagctgtccccgcctcctagtcgctagcgggcggggggtgggggggaaaaatgcggccaggaaagattccagccaatcaaaacactcagcgcctccgcgcggactagggcaaagccaacggagtcccgcccctccaccgaggcgcagatgccgaatctaaggcgtcggattggtcagcgcggcgtggtgggcgggttaaggggcgtggctccaggagtggagtatatcaggcgggactcttgcgctccccctcgcactcgcgccttaggaagcttgggtgtgtgtggcgcgctgtcttcccgctcgcgtcagggacctgcccgactcagcggtgagggagatctggcctcggcggccaagcttggcaatccggtactgttggtaaagccaccatggatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtgatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgactctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 20 - DNA, 7557 bp, synthetic construct
  source 1..7557: mol_type = other DNA
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccgaggagatctgccgccgcgatcgccttccccgccgttccccgccgttccccgccgttccccgccgttccccgccgttccccgccgctaacttcgaccgtcaagccccctcggttccagcatcctgttttgtaaaagccatctcaggcagtctattggtgagaaaattaaggggctggcttcacgccagcagtcagacaagggtgagggctgaagtcagagatgagatggaatgacgccagggaaaagggtcccatgacaggcggctagatgttgagcacagaggagaaagagtaggagtcagggtcaaaccttaaaacgttgggtctgacatcatggactgtgaagggtgatgacatcagtgacggtgggggataaaatgtcaggcctttaaagggtatgaggttaaagaactagaaggtgaaggaggttaacatcagagaatggcagggacctagaggaagacgactacgatggtgaggaagacatccgatccagcgggggaaatgatggcaaggacgggaggatgatgtcagggacagcaagtagtatgagattgagaaggttcctttgtggaagggataaaggcagctcagccggttgtgggggggatgctgacgtttagggtgcaccagctgttcaaaagtcgaccccatttccggaaacaccccatttccggaaacaccccatttcgcggagaacactgctcccttcgctttgctgtcccttcctctccccaccactccgctcctgctggcctcagccaatcagcacgcacgccgggacgcgcaaggggaacgttccgggacgttctcgctacgtactctttatcaatcgtcttccggcgcagccccgtccctgttttttgtgctcctccgagctcgctgttcgtccgggttttttacgttttaatttccaggtaagttgcatatgtcctgcgaaacggcggctcctcgccacacacgcgccgcgccggccgtgaccggggaatagcgagtggggagagtaatggccactgacacgtacgcgtacgcggccgctcgagatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagagtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggccgaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 21 - DNA, 5313 bp, synthetic construct
  source 1..5313: mol_type = other DNA
ggcctaactggccggtacctgagctcgctagcgcaacttagtctcacacgccttggagagcaagcgagtcttgccattggataattccaccgtctttcttctgcaagtccctcctttccccctccctcattgggcggggcagcagagaaggggcggggcctaggttgggcttgtggcgcgctgctccctcctccttacccccccctccctgtccggtccgggttcgcttgcctcgtcagcgtccgcgtttttcccggccccccccaacccccccggacaggacccccttgagcctcgaggatatcaagatctggcctcggcggccaagcttggcaatccggtactgttggtaaagccaccatggatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtgatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgactctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 22 - DNA, 5392 bp, synthetic construct
  source 1..5392: mol_type = other DNA
ggcctaactggccggtaccttccccgccgttccccgccgttccccgccgttccccgccgttccccgccgtctggctaactagagaacccactgcgccacgagctcgctagcgcaacttagtctcacacgccttggagagcaagcgagtcttgccattggataattccaccgtctttcttctgcaagtccctcctttccccctccctcattgggcggggcagcagagaaggggcggggcctaggttgggcttgtggcgcgctgctccctcctccttacccccccctccctgtccggtccgggttcgcttgcctcgtcagcgtccgcgtttttcccggccccccccaacccccccggacaggacccccttgagcctcgaggatatcaagatctggcctcggcggccaagcttggcaatccggtactgttggtaaagccaccatggatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtgatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgactctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 23 - DNA, 8959 bp, synthetic construct
  source 1..8959: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgttccccgccgttccccgccgttccccgccgttccccgccgttccccgccgttccccgccgttccccgccgttccccgccgtctggtggcggaggctcgggcggaggtgggtcgggtggcggcggatcagctgggaaaactgggacgtcggttgtattggcaagaatatcacgacaggtgtcggggttcagatgaagttgggatgtctgacagccgggtcttggttgggtaggggatctgggctaggttgttatgtcgggactgctggagcacaggcaaagtcggggtcccagtgacacccagaagtccccatgaggcctgaggcctgggaatcggggctagaggcccatttgttccccgccgcaggccagggagcaggcacgcgtaactgggtcagccccgccccgccccgccgcgcgctggtcacgccccctcgagcgcgggtggaggactgcggactgcaaacgtctcggccaatagtcgagcccgacaccagtccgcgggagggggcggaaccgagatagactcggcccccagctcatgaatataagacgcaggcgcgccctctgattggcccaggatgtgtagggggcggggcccggcggaagcgtatataaggccgggctcggggacgcccccccctcactcgcgcgttaggaggctcgggtcgttgtggtgcgctgtcttcccgcttgcgtcagggacctgcccgactcagtggtgagggccccaagtttgtacaaaaaagcaggctgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 24 - DNA, 9235 bp, synthetic construct
  source 1..9235: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgttccccgccgttccccgccgttccccgccgttccccgccgttccccgccgtctggtggcggaggctcgggcggaggtgggtcgggtggcggcggatcacagcatcctgttttgtaaaagccatctcaggcagtctattggtgagaaaattaaggggctggcttcacgccagcagtcagacaagggtgagggctgaagtcagagatgagatggaatgacgccagggaaaagggtcccatgacaggcggctagatgttgagcacagaggagaaagagtaggagtcagggtcaaaccttaaaacgttgggtctgacatcatggactgtgaagggtgatgacatcagtgacggtgggggataaaatgtcaggcctttaaagggtatgaggttaaagaactagaaggtgaaggaggttaacatcagagaatggcagggacctagaggaagacgactacgatggtgaggaagacatccgatccagcgggggaaatgatggcaaggacgggaggatgatgtcagggacagcaagtagtatgagattgagaaggttcctttgtggaagggataaaggcagctcagccggttgtgggggggatgctgacgtttagggtgcaccagctgttcaaaagtcgaccccatttccggaaacaccccatttccggaaacaccccatttcgcggagaacactgctcccttcgctttgctgtcccttcctctccccaccactccgctcctgctggcctcagccaatcagcacgcacgccgggacgcgcaaggggaacgttccgggacgttctcgctacgtactctttatcaatcgtcttccggcgcagccccgtccctgttttttgtgctcctccgagctcgctgttcgtccgggttttttacgttttaatttccaggtaagttgcatatgtcctgcgaaacggcggctcctcgccacacacgcgccgcgccggccgtgaccggggaatagcgagtggggagagtaatggccactgacacgtcaagtttgtacaaaaaagcaggctgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 25 - DNA, 8590 bp, synthetic construct
  source 1..8590: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgttccccgccgttccccgccgttccccgccgttccccgccgttccccgccgtctggtggcggaggctcgggcggaggtgggtcgggtggcggcggatcaggttgactggtttccttccaagccaatcatctccagctcccgcccatcttcacttcctgcatccttcattggcttttaacactgagagggcggtctttttaggcggacaccaggcacgcaacttagtctcacacgccttggagagcaagcgagtcttgccattggataattccaccgtctttcttctgcaagtccctcctttccccctccctcattgggcggggcagcagagaaggggcggggcctaggttgggcttgtggccaagtttgtacaaaaaagcaggctgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 26 - DNA, 1323 bp, synthetic construct
  source 1..1323: mol_type = other DNA
catctgtcttctggcttcacaaatgcgcctcagttaaataactgtaattaaatattaaaagaaaaaaaagaacataaaagtgttgagtcgatgagtcaaggttggagccatatttgggagtctcattgaggtagcaaaagctctttgttccccgccgcaggacaaacgggtcgggaacgcggaagtgggtcagctccgcctccaaatcagctgtccccgcctcctagtcgctagcgggcggggggtgggggggaaaaatgcggccaggaaagattccagccaatcaaaacactcagcgcctccgcgcggactagggcaaagccaacggagtcccgcccctccaccgaggcgcagatgccgaatctaaggcgtcggattggtcagcgcggcgtggtgggcgggttaaggggcgtggctccaggagtggagtatatcaggcgggactcttgcgctccccctcgcactcgcgccttaggaagcttgggtgtgtgtggcgcgctgtcttcccgctcgcgtcagggacctgcccgactcagcggtgagggagatctggcctcggcggccaagcttggcaatccggtactgttggtaaagccaccatggatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtga

SEQ ID NO: 27 - DNA, 1409 bp, synthetic construct
  source 1..1409: mol_type = other DNA
ttccccgccgttccccgccgttccccgccgttccccgccgttccccgccgtctggctaactagagaacccactgcgccacagtacccatctgtcttctggcttcacaaatgcgcctcagttaaataactgtaattaaatattaaaagaaaaaaaagaacataaaagtgttgagtcgatgagtcaaggttggagccatatttgggagtctcattgaggtagcaaaagctctttgttccccgccgcaggacaaacgggtcgggaacgcggaagtgggtcagctccgcctccaaatcagctgtccccgcctcctagtcgctagcgggcggggggtgggggggaaaaatgcggccaggaaagattccagccaatcaaaacactcagcgcctccgcgcggactagggcaaagccaacggagtcccgcccctccaccgaggcgcagatgccgaatctaaggcgtcggattggtcagcgcggcgtggtgggcgggttaaggggcgtggctccaggagtggagtatatcaggcgggactcttgcgctccccctcgcactcgcgccttaggaagcttgggtgtgtgtggcgcgctgtcttcccgctcgcgtcagggacctgcccgactcagcggtgagggagatctggcctcggcggccaagcttggcaatccggtactgttggtaaagccaccatggatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtga

SEQ ID NO: 28 - DNA, 1714 bp, synthetic construct
  source 1..1714: mol_type = other DNA
ttccccgccgttccccgccgttccccgccgttccccgccgttccccgccgttccccgccgctaacttcgaccgtcaagccccctcggttccagcatcctgttttgtaaaagccatctcaggcagtctattggtgagaaaattaaggggctggcttcacgccagcagtcagacaagggtgagggctgaagtcagagatgagatggaatgacgccagggaaaagggtcccatgacaggcggctagatgttgagcacagaggagaaagagtaggagtcagggtcaaaccttaaaacgttgggtctgacatcatggactgtgaagggtgatgacatcagtgacggtgggggataaaatgtcaggcctttaaagggtatgaggttaaagaactagaaggtgaaggaggttaacatcagagaatggcagggacctagaggaagacgactacgatggtgaggaagacatccgatccagcgggggaaatgatggcaaggacgggaggatgatgtcagggacagcaagtagtatgagattgagaaggttcctttgtggaagggataaaggcagctcagccggttgtgggggggatgctgacgtttagggtgcaccagctgttcaaaagtcgaccccatttccggaaacaccccatttccggaaacaccccatttcgcggagaacactgctcccttcgctttgctgtcccttcctctccccaccactccgctcctgctggcctcagccaatcagcacgcacgccgggacgcgcaaggggaacgttccgggacgttctcgctacgtactctttatcaatcgtcttccggcgcagccccgtccctgttttttgtgctcctccgagctcgctgttcgtccgggttttttacgttttaatttccaggtaagttgcatatgtcctgcgaaacggcggctcctcgccacacacgcgccgcgccggccgtgaccggggaatagcgagtggggagagtaatggccactgacacgtacgcgtacgcggccgctcgagatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaa

SEQ ID NO: 29 - DNA, 1052 bp, synthetic construct
  source 1..1052: mol_type = other DNA
gcaacttagtctcacacgccttggagagcaagcgagtcttgccattggataattccaccgtctttcttctgcaagtccctcctttccccctccctcattgggcggggcagcagagaaggggcggggcctaggttgggcttgtggcgcgctgctccctcctccttacccccccctccctgtccggtccgggttcgcttgcctcgtcagcgtccgcgtttttcccggccccccccaacccccccggacaggacccccttgagcctcgaggatatcaagatctggcctcggcggccaagcttggcaatccggtactgttggtaaagccaccatggatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtga

SEQ ID NO: 30 - DNA, 1144 bp, synthetic construct
  source 1..1144: mol_type = other DNA
ttccccgccgttccccgccgttccccgccgttccccgccgttccccgccgtctggctaactagagaacccactgcgccacgagctcgctagcgcaacttagtctcacacgccttggagagcaagcgagtcttgccattggataattccaccgtctttcttctgcaagtccctcctttccccctccctcattgggcggggcagcagagaaggggcggggcctaggttgggcttgtggcgcgctgctccctcctccttacccccccctccctgtccggtccgggttcgcttgcctcgtcagcgtccgcgtttttcccggccccccccaacccccccggacaggacccccttgagcctcgaggatatcaagatctggcctcggcggccaagcttggcaatccggtactgttggtaaagccaccatggatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtga

SEQ ID NO: 31 - DNA, 1469 bp, synthetic construct
  source 1..1469: mol_type = other DNA
ttccccgccgttccccgccgttccccgccgttccccgccgttccccgccgttccccgccgttccccgccgttccccgccgtctggtggcggaggctcgggcggaggtgggtcgggtggcggcggatcagctgggaaaactgggacgtcggttgtattggcaagaatatcacgacaggtgtcggggttcagatgaagttgggatgtctgacagccgggtcttggttgggtaggggatctgggctaggttgttatgtcgggactgctggagcacaggcaaagtcggggtcccagtgacacccagaagtccccatgaggcctgaggcctgggaatcggggctagaggcccatttgttccccgccgcaggccagggagcaggcacgcgtaactgggtcagccccgccccgccccgccgcgcgctggtcacgccccctcgagcgcgggtggaggactgcggactgcaaacgtctcggccaatagtcgagcccgacaccagtccgcgggagggggcggaaccgagatagactcggcccccagctcatgaatataagacgcaggcgcgccctctgattggcccaggatgtgtagggggcggggcccggcggaagcgtatataaggccgggctcggggacgcccccccctcactcgcgcgttaggaggctcgggtcgttgtggtgcgctgtcttcccgcttgcgtcagggacctgcccgactcagtggtgagggccccaagtttgtacaaaaaagcaggctgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagta

SEQ ID NO: 32 - DNA, 1746 bp, synthetic construct
  source 1..1746: mol_type = other DNA
ttccccgccgttccccgccgttccccgccgttccccgccgttccccgccgtctggtggcggaggctcgggcggaggtgggtcgggtggcggcggatcacagcatcctgttttgtaaaagccatctcaggcagtctattggtgagaaaattaaggggctggcttcacgccagcagtcagacaagggtgagggctgaagtcagagatgagatggaatgacgccagggaaaagggtcccatgacaggcggctagatgttgagcacagaggagaaagagtaggagtcagggtcaaaccttaaaacgttgggtctgacatcatggactgtgaagggtgatgacatcagtgacggtgggggataaaatgtcaggcctttaaagggtatgaggttaaagaactagaaggtgaaggaggttaacatcagagaatggcagggacctagaggaagacgactacgatggtgaggaagacatccgatccagcgggggaaatgatggcaaggacgggaggatgatgtcagggacagcaagtagtatgagattgagaaggttcctttgtggaagggataaaggcagctcagccggttgtgggggggatgctgacgtttagggtgcaccagctgttcaaaagtcgaccccatttccggaaacaccccatttccggaaacaccccatttcgcggagaacactgctcccttcgctttgctgtcccttcctctccccaccactccgctcctgctggcctcagccaatcagcacgcacgccgggacgcgcaaggggaacgttccgggacgttctcgctacgtactctttatcaatcgtcttccggcgcagccccgtccctgttttttgtgctcctccgagctcgctgttcgtccgggttttttacgttttaatttccaggtaagttgcatatgtcctgcgaaacggcggctcctcgccacacacgcgccgcgccggccgtgaccggggaatagcgagtggggagagtaatggccactgacacgtcaagtttgtacaaaaaagcaggctgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 33 - DNA, 1101 bp, synthetic construct
  source 1..1101: mol_type = other DNA
ttccccgccgttccccgccgttccccgccgttccccgccgttccccgccgtctggtggcggaggctcgggcggaggtgggtcgggtggcggcggatcaggttgactggtttccttccaagccaatcatctccagctcccgcccatcttcacttcctgcatccttcattggcttttaacactgagagggcggtctttttaggcggacaccaggcacgcaacttagtctcacacgccttggagagcaagcgagtcttgccattggataattccaccgtctttcttctgcaagtccctcctttccccctccctcattgggcggggcagcagagaaggggcggggcctaggttgggcttgtggccaagtttgtacaaaaaagcaggctgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 34 - DNA, 22 bp, synthetic construct
  source 1..22: mol_type = other DNA
gcactgtgcgcaaagacctcca

SEQ ID NO: 35 - DNA, 22 bp, synthetic construct
  source 1..22: mol_type = other DNA
ggaaaccaggccaggttctgcc

SEQ ID NO: 36 - DNA, 22 bp, synthetic construct
  source 1..22: mol_type = other DNA
ctgggcacaggacctggtggta

SEQ ID NO: 37 - DNA, 22 bp, synthetic construct
  source 1..22: mol_type = other DNA
ggctgccaaccgacattctgca

SEQ ID NO: 38 - DNA, 545 bp, synthetic construct
  source 1..545: mol_type = other DNA
catctgtcttctggcttcacaaatgcgcctcagttaaataactgtaattaaatattaaaagaaaaaaaagaacataaaagtgttgagtcgatgagtcaaggttggagccatatttgggagtctcattgaggtagcaaaagctctttgttccccgccgcaggacaaacgggtcgggaacgcggaagtgggtcagctccgcctccaaatcagctgtccccgcctcctagtcgctagcgggcggggggtgggggggaaaaatgcggccaggaaagattccagccaatcaaaacactcagcgcctccgcgcggactagggcaaagccaacggagtcccgcccctccaccgaggcgcagatgccgaatctaaggcgtcggattggtcagcgcggcgtggtgggcgggttaaggggcgtggctccaggagtggagtatatcaggcgggactcttgcgctccccctcgcactcgcgccttaggaagcttgggtgtgtgtggcgcgctgtcttcccgctcgcgtcagggacctgcccgactcagcggtgaggg

SEQ ID NO: 39 - DNA, 907 bp, synthetic construct
  source 1..907: mol_type = other DNA
cagcatcctgttttgtaaaagccatctcaggcagtctattggtgagaaaattaaggggctggcttcacgccagcagtcagacaagggtgagggctgaagtcagagatgagatggaatgacgccagggaaaagggtcccatgacaggcggctagatgttgagcacagaggagaaagagtaggagtcagggtcaaaccttaaaacgttgggtctgacatcatggactgtgaagggtgatgacatcagtgacggtgggggataaaatgtcaggcctttaaagggtatgaggttaaagaactagaaggtgaaggaggttaacatcagagaatggcagggacctagaggaagacgactacgatggtgaggaagacatccgatccagcgggggaaatgatggcaaggacgggaggatgatgtcagggacagcaagtagtatgagattgagaaggttcctttgtggaagggataaaggcagctcagccggttgtgggggggatgctgacgtttagggtgcaccagctgttcaaaagtcgaccccatttccggaaacaccccatttccggaaacaccccatttcgcggagaacactgctcccttcgctttgctgtcccttcctctccccaccactccgctcctgctggcctcagccaatcagcacgcacgccgggacgcgcaaggggaacgttccgggacgttctcgctacgtactctttatcaatcgtcttccggcgcagccccgtccctgttttttgtgctcctccgagctcgctgttcgtccgggttttttacgttttaatttccaggtaagttgcatatgtcctgcgaaacggcggctcctcgccacacacgcgccgcgccggccgtgaccggggaatagcgagtggggagagtaatggccactgacacgt

SEQ ID NO: 40 - DNA, 261 bp, synthetic construct
  source 1..261: mol_type = other DNA
gcaacttagtctcacacgccttggagagcaagcgagtcttgccattggataattccaccgtctttcttctgcaagtccctcctttccccctccctcattgggcggggcagcagagaaggggcggggcctaggttgggcttgtggcgcgctgctccctcctccttacccccccctccctgtccggtccgggttcgcttgcctcgtcagcgtccgcgtttttcccggccccccccaacccccccggacaggacccccttgagc

SEQ ID NO: 41 - DNA, 48 bp, synthetic construct
  source 1..48: mol_type = other DNA
tctggtggcggaggctcgggcggaggtgggtcgggtggcggcggatca

SEQ ID NO: 42 - DNA, 30 bp, synthetic construct
  source 1..30: mol_type = other DNA
caagtttgtacaaaaaagcaggctgccacc

SEQ ID NO: 43 - DNA, 42 bp, synthetic construct
  source 1..42: mol_type = other DNA
tctggctaactagagaacccactgcgccacgagctcgctagc

SEQ ID NO: 44 - DNA, 71 bp, synthetic construct
  source 1..71: mol_type = other DNA
ctcgaggatatcaagatctggcctcggcggccaagcttggcaatccggtactgttggtaaagccaccatgg

SEQ ID NO: 45 - DNA, 33 bp, synthetic construct
  source 1..33: mol_type = other DNA
ccgctaacttcgaccgtcaagccccctcggttc

SEQ ID NO: 46 - DNA, 21 bp, synthetic construct
  source 1..21: mol_type = other DNA
acgcgtacgcggccgctcgag

SEQ ID NO: 47 - DNA, 36 bp, synthetic construct
  source 1..36: mol_type = other DNA
tctggctaactagagaacccactgcgccacagtacc

SEQ ID NO: 48 - DNA, 58 bp, synthetic construct
  source 1..58: mol_type = other DNA
agatctggcctcggcggccaagcttggcaatccggtactgttggtaaagccaccatgg